(19) United States
(12) Patent Application Publication
(10) Pub. No.: US 2025/0290100 A1
(43) Pub. Date: Sep. 18, 2025

(54) SINGLE pegRNA-MEDIATED LARGE INSERTIONS

(71) Applicant: University of Massachusetts, Boston, MA (US)

(72) Inventors: Chunwei Zheng, Worcester, MA (US); Wen Xue, Natick, MA (US); Erik Sontheimer, Worcester, MA (US); Bin Liu, Worcester, MA (US); Xiaolong Dong, Worcester, MA (US)

(73) Assignee: University of Massaachusetts, Boston, MA (US)

(21) Appl. No.: 18/860,100

(22) PCT Filed: Apr. 26, 2023
(86) PCT No.: PCT/US2023/066238

§ 371 Date: Oct. 25, 2024

Related U.S. Application Data
(60) Provisional application No. 63/334,956, filed on Apr. 26, 2022.

(51) Int. Cl. C12N 15/90; C12N 9/22; C12N 15/11; C12N 15/86

(52) U.S. Cl. CPC C12N 15/907; C12N 9/226; C12N 15/111; C12N 15/86; C12N 2310/20; C12N 2310/3519; C12N 2310/532; C12N 2750/14143

(57) ABSTRACT

The invention described herein provide methods and systems for deleting or inserting long (e.g., >100-500 bp) DNA sequences into a target DNA sequence using a single prime editing guide RNA (pegRNA) in conjunction with a CRISPR/Cas DNA nuclease.a

### REFERENCE TO RELATED APPLICATIONS

This application claims priority to and the benefit of the filing date of U.S. Provisional Patent Application No. 63/334,956, filed on Apr. 26, 2022, the entire contents of which are incorporated herein by reference.

### GOVERNMENT SUPPORT

This invention was made with government support under HL131471, TR002668, HL137167, HL147367, GM115911, and HL158506 awarded by the National Institutes of Health. The government has certain rights in the invention.

### REFERENCE TO THE SEQUENCE LISTING

The instant application contains a Sequence Listing XML file, which has been submitted electronically in XML format and is hereby incorporated by reference in its entirety. Said XML copy, created on Apr. 26, 2023, is named 122274_08020_SL.xml and is 133,541 bytes in size.

### BACKGROUND OF THE INVENTION

Correction of genetic mutations in vivo has broad potential therapeutic application for a range of human genetic diseases. Prime editors (PE) composed of a Cas9 nickase and engineered reverse transcriptase have enabled precise nucleotide changes, sequence insertions and deletions (Anzalone et al., Search-and-replace genome editing without double-strand breaks or donor DNA. Nature 576:149-157, 2019). This innovative technology does not induce double-stranded DNA breaks (DSBs) and does not require a donor DNA template in conjunction with homology directed repair to introduce precise sequence changes into the genome.

In particular, prime editing (PE) enables insertion, deletion, and/or replacement of genomic DNA sequences without requiring error-prone double-strand DNA (DBS) breaks. Prime editing utilizes an engineered Cas9 nickase-reverse transcriptase fusion protein (called PE1 or PE2), paired with an engineered prime editing guide RNA (pegRNA) that both directs the engineered Cas9 nickase to the target genomic site and encodes the information for the desired edit. More specifically, prime editing comprises multiple steps including: 1) the Cas9 nickase domain binds and nicks the target genomic DNA site, which is specified by the pegRNA's spacer sequence; 2) the reverse transcriptase domain uses the nicked genomic DNA as a primer to initiate the synthesis of an edited DNA strand using an engineered extension on the pegRNA as a template for reverse transcription. This generates a single-stranded 3′ flap containing the edited DNA sequence; 3) cellular DNA repair resolves the 3′ flap intermediate by the displacement of a 5′ flap species that occurs via invasion by the edited 3′ flap, excision of the 5′ flap containing the original DNA sequence, and ligation of the new 3′ flap to incorporate the edited DNA strand, forming a heteroduplex of one edited and one unedited strand; and 4) cellular DNA repair replaces the unedited strand within the heteroduplex using the edited strand as a template for repair, completing the editing process.

Genomic insertions, duplications, and insertion/deletions (indels) account for ˜14% of human pathogenic mutations. Current gene editing methods cannot efficiently insert long DNA sequence. The recently developed TwinPE technology uses two pegRNAs. Specifically, the TwinPE systems target genomic DNA sequences that contain two protospacer sequences on opposite strands of the genomic DNA. PE2·pegRNA complexes target each protospacer, generate a single-stranded nick, and reverse transcribe the pegRNA-encoded template containing the desired insertion sequence. After synthesis and release of the 3′ DNA flaps, a hypothetical intermediate exists possessing annealed 3′ flaps containing the edited DNA sequence and annealed 5′ flaps containing the original DNA sequence. Excision of the original DNA sequence contained in the 5′ flaps, followed by ligation of the 3′ flaps to the corresponding excision sites, generates the desired edited product. See Anzalone et al., Programmable large DNA deletion, replacement, integration, and inversion with twin prime editing and site-specific recombinases. bioRxiv preprint doi: doi.org/10.1101/2021.11.01.466790, November 2021.

However, TwinPE is not especially efficient for large insertions (>100 bp). Thus, developing a method to accurately insert longer sequences is needed.

### SUMMARY OF THE INVENTION

Described herein is a method and system (hereinafter “bidirectional pegRNA” or “biPE” for short, or “Template-jumping Prime Editing” or “TJ-PE” for short) for either deleting or inserting long (e.g., >50, 100, 200, 300, 400, 500, 600, 700, 800, 900 or more bp) DNA sequences into a target location, such as a target location in a host genome, or both, without introducing double stranded breaks (DSBs), using a single pegRNA in conjunction with a CRISPR/Cas DNA nuclease/nickase.

The method and system of the invention, compared to the existing TwinPE method that utilizes two pegRNA, is, among other things, more cost effective, and can reduce the cost in RNA synthesis.

The method and system of the invention find broad usage in cells and in vivo, and has use in a number of therapeutic applications to treat diseases and indications treatable by prime-editing. The method and system of the invention are briefly described in the following numbered paragraphs:
1. A prime editing guide RNA (pegRNA), comprising, from 5′ to 3′:
   (1) a single guide RNA (sgRNA);
   (2) a second primer binding sequence (2^{nd} PBS);
   (3) an optional reverse transcription template (RTT) sequence; and,
   (4) a first primer binding sequence (1^{st} PBS);
   or a split variant combination (SVC) thereof, wherein the SVC comprises:
   (a) the sgRNA; and,
   (b) a prime editing template RNA (petRNA) comprising, from 5′ to 3′, (2)-(4), wherein the petRNA further comprises a linked aptamer (such as MS2) that specifically binds an aptamer binding protein (such as MCP or a functional fragment thereof that binds MS2);
   wherein:
   (i) the sgRNA is capable of forming a complex with a CRISPR/Cas nickase and targeting the complex to a target (e.g., target genomic) DNA sequence through base pairing with a targeting strand of the target (genomic) DNA sequence to enable nicking of the non-targeting strand reverse complementary to the targeting strand by the CRISPR/Cas nickase;
   (ii) the 1^{st} PBS is capable of annealing with the 3′ end of the nicked non-targeting strand created by the CRISPR/Cas nickase, to prime reverse transcription of the RTT (if present) and the 2^{nd} PBS by a reverse transcriptase (RT); and,
   (iii) the reverse transcription product of the 2^{nd} PBS is capable of annealing to an anchor sequence on the targeting strand, wherein nicking the targeting strand 3′ to the anchor sequence (e.g., by the CRISPR/Cas nickase and a nicking sgRNA) creates a 3′ end of the targeting strand capable of being extended by the RT to form a second strand cDNA, using the reverse transcribed RTT (if present) and the 1^{st} PBS as template;
   or wherein:
   (A) the sgRNA is capable of forming a complex with the CRISPR/Cas nickase and targeting the complex to the target (e.g., target genomic) DNA sequence through base pairing with the targeting strand of the target (genomic) DNA sequence to enable nicking of the non-targeting strand reverse complementary to the targeting strand by the CRISPR/Cas nickase;
   (B) the 1^{st} PBS is capable of annealing with the 3′ end of the anchor sequence on the targeting strand (resulting from nicking by the CRISPR/Cas nickase and the nicking sgRNA) to prime reverse transcription of the RTT (if present) and the 2^{nd} PBS by the RT; and,
   (C) the reverse transcription product of the 2^{nd} PBS is capable of annealing to the 3′ end of the nicked non-targeting strand created by the CRISPR/Cas nickase to enable the RT to synthesize a second strand cDNA, using the reverse transcribed RTT (if present) and the 1^{st} PBS as template;
   wherein the reverse complement sequence of the anchor sequence on the non-targeting strand is either upstream (5′) or downstream (3′) of the 1^{st} PBS binding sequence;
   optionally, the RT is fused to the CRISPR/Cas nickase, and/or optionally, the RT is fused to the aptamer binding protein.
2. The pegRNA or SVC of paragraph 1, wherein:
   (a) the sgRNA is about 80-120 (e.g., 90-110, or about 100) nucleotides in length;
   (b) the 1^{st} PBS is about 10-20 (e.g., 12-18 or about 15) nucleotides in length;
   (c) the optional RTT is about 0-900 (e.g., 0-800, 0-850, 5-550, 10-500, 15-400, 20-300, 50-200, 30-60, 40-50, 80-150, 100-120, 0-5, 0, 100, 200, 300, 400, 500, 600, 700, 800, or about 900) nucleotides in length; and/or,
   (d) the 2^{nd} PBS is about 10-20 (e.g., 12-18 or about 15) nucleotides in length.
3. The pegRNA or SVC of paragraph 1 or 2, further comprising a linker between the 1^{st} PBS and the RTT, between the RTT and the 2^{nd} PBS, and/or (in the pegRNA) between the 2^{nd} PBS and the sgRNA.
4. The pegRNA or SVC of paragraph 3, wherein the linker is independently 1, 2, 3, 4, 5, 6, 7, 8, 9, or 10 nucleotides in length.
5. The pegRNA or SVC of any one of paragraphs 1-4, wherein the CRISPR/Cas nickase is a Class 2, Type II Cas effector enzyme (e.g., a Cas9, such as SpCas9, SpCas9-HF1, eSpCas9, SaCas9, SaCas9-HF, KKHSaCas9, StCas9, NmCas9, FnCas9, CjCas9, ScCas9, HypaCas9, xCas9, SpRY, SpG, or SauriCas9) lacking (HNH) endonuclease activity against the targeting strand.
6. The pegRNA or SVC of any one of paragraphs 1-5, wherein the CRISPR/Cas nickase lacks endonuclease activity against the non-targeting strand, when forming a complex with the nicking sgRNA to nick the targeting strand (immediately) 3′ to the anchor sequence.
7. The pegRNA or SVC of any one of paragraphs 1-6, wherein the nicking site of the non-targeting strand and the nicking site of the targeting strand are separated by 5, 10, 15, 20, 25, 30, 35, 40, 45, 50, 55, 60, 65, 70, 75, 80, 85, 90, 95, 100, 105, 110, 120, 130, 140, 150, 160, 170, 180, 190, 200, 210, 220, 230, 240, 250, 260, 270, 280, 290, or 300 nucleotides, with the nicking site of the non-targeting strand being either 5′ or 3′ to the nicking site of the targeting strand.
8. The pegRNA or SVC of any one of paragraphs 1-7, wherein the 1^{st} PBS is linked to an RNA element that enhances pegRNA or petRNA stability, and/or improves prime editing efficiency; optionally, the RNA element comprises a trimmed evopreQ1 (tevopreQ1) motif or an aptamer such as MS2.
9. The pegRNA or SVC of any one of paragraphs 1-8, wherein the petRNA is circular, and/or wherein the linked aptamer (such as MS2) is immediately 5′ to the 2^{nd} PBS.
10. The pegRNA or SVC of paragraph 9, wherein the circular petRNA is generated by in vitro transcription to generate a precursor RNA that is circularized post transcription via self-splicing through a permuted group I catalytic intron.
11. A prime editing guide RNA (pegRNA), comprising, from 5′ to 3′:
   (1) a second primer binding sequence (2^{nd} PBS);
   (2) an optional reverse transcription template (RTT) sequence;
   (3) a first primer binding sequence (1^{st} PBS); and,
   (4) a single guide RNA (sgRNA);
   wherein:
   (i) the sgRNA is capable of forming a complex with a CRISPR/Cas nickase and targeting the complex to a target (e.g., a target genomic) DNA sequence through base pairing with a targeting strand of the target (genomic) DNA sequence to enable nicking of the non-targeting strand reverse complementary to the targeting strand by the CRISPR/Cas nickase;
   (ii) the 1^{st} PBS is capable of annealing with the 3′ end of the nicked non-targeting strand created by the CRISPR/Cas nickase, to prime reverse transcription of the RTT (if present) and the 2^{nd} PBS by a reverse transcriptase (RT);
      optionally, the RT is fused to the CRISPR/Cas nickase; and,
   (iii) the reverse transcription product of the 2^{nd} PBS is capable of annealing to an anchor sequence on the targeting strand, wherein nicking the targeting strand 3′ to the anchor sequence (e.g., by the CRISPR/Cas nickase and a nicking sgRNA) creates a 3′ end of the targeting strand capable of being extended by the RT to form a second strand cDNA, using the reverse transcribed RTT (if present) and the 1^{st} PBS as template;
   or wherein:
   (A) the sgRNA is capable of forming a complex with the CRISPR/Cas nickase and targeting the complex to the target (e.g., target genomic) DNA sequence through base pairing with the targeting strand of the target (genomic) DNA sequence to enable nicking of the non-targeting strand reverse complementary to the targeting strand by the CRISPR/Cas nickase;
   (B) the 1^{st} PBS is capable of annealing with the 3′ end of the anchor sequence on the targeting strand (resulting from nicking by the CRISPR/Cas nickase and the nicking sgRNA) to prime reverse transcription of the RTT (if present) and the 2^{nd} PBS by the RT; and,
   (C) the reverse transcription product of the 2^{nd} PBS is capable of annealing to the 3′ end of the nicked non-targeting strand created by the CRISPR/Cas nickase to enable the RT to synthesize a second strand cDNA, using the reverse transcribed RTT (if present) and the 1^{st} PBS as template;
   wherein the reverse complement sequence of the anchor sequence on the non-targeting strand is either upstream (5′) or downstream (3′) of the 1^{st} PBS binding sequence.
12. The pegRNA of paragraph 11, wherein:
   (a) the sgRNA is about 80-120 (e.g., 90-110, or about 100) nucleotides in length;
   (b) the 1^{st} PBS is about 10-20 (e.g., 12-18 or about 15) nucleotides in length;
   (c) the optional RTT is about 0-900 (e.g., 0-800, 0-850, 5-550, 10-500, 15-400, 20-300, 50-200, 30-60, 40-50, 80-150, 100-120, 0-5, 0, 100, 200, 300, 400, 500, 600, 700, 800, or about 900) nucleotides in length; and/or,
   (d) the 2^{nd} PBS is about 10-20 (e.g., 12-18 or about 15) nucleotides in length.
13. The pegRNA of paragraph 11 or 12, further comprising a linker between the 1^{st} PBS and the RTT, between the RTT and the 2^{nd} PBS, and/or between the 2^{nd} PBS and the sgRNA.
14. The pegRNA of paragraph 13, wherein the linker is independently 1, 2, 3, 4, 5, 6, 7, 8, 9, or 10 nucleotides in length.
15. The pegRNA of any one of paragraphs 11-14, wherein the CRISPR/Cas nickase is a Class 2, Type V Cas effector enzyme (e.g., Cas12a/Cpf1, Cas12b, Cas12c, Cas12d, Cas12e/CasX, Cas12f/Cas14, Cas12g, Cas12h, Cas12i, Cas12k, or V-U) lacking endonuclease activity against the targeting strand.
16. The pegRNA of any one of paragraphs 11-15, wherein the CRISPR/Cas nickase lacks endonuclease activity against the non-targeting strand, when forming a complex with the nicking sgRNA to nick the targeting strand (immediately) 3′ to the anchor sequence.
17. The pegRNA of any one of paragraphs 11-16, wherein the nicking site of the non-targeting strand and the nicking site of the targeting strand are separated by 5, 10, 15, 20, 25, 30, 35, 40, 45, 50, 55, 60, 65, 70, 75, 80, 85, 90, 95, 100, 105, 110, 120, 130, 140, 150, 160, 170, 180, 190, 200, 210, 220, 230, 240, 250, 260, 270, 280, 290, or 300 nucleotides, with the nicking site of the non-targeting strand being either 5′ or 3′ to the nicking site of the targeting strand.
18. A complex comprising:
   (1) the pegRNA or SVC of any one of paragraphs 1-10 (or the pegRNA of any one of paragraphs 11-17); and,
   (2) the CRISPR/Cas nickase of any one of paragraphs 1-10 (or the pegRNA of any one of paragraphs 11-17).
19. The complex of paragraph 18, further comprising:
   (3) a target (e.g., a target genomic) DNA sequence, wherein the target (genomic) DNA sequence base pairs with the sgRNA through a targeting strand of the target (genomic) DNA sequence.
20. The complex of paragraph 19, further comprising:
   (4) a reverse transcribed first strand cDNA reverse complementary in sequence to the 2^{nd} PBS and the RTT sequence (if present); and optionally,
   (5) a reverse transcribed second strand cDNA reverse complementary in sequence to the first strand cDNA.
21. A method of inserting a donor DNA sequence into/around/proximate to a target (e.g., a target genomic) DNA sequence, the method comprising contacting the target (genomic) DNA sequence with: (1) the pegRNA or the SVC, (2) the CRISPR/Cas nickase, and (3) the nicking sgRNA, of any one of paragraphs 1-10 (or 11-17), to permit the synthesis of a first strand cDNA and a second strand cDNA based on the RTT sequence of the pegRNA or SVC, through the reverse transcriptase (RT), wherein the RTT sequence encodes the donor DNA sequence.
22. The method of paragraph 21, wherein the method is carried out in vitro.
23. The method of paragraph 21, wherein the method is carried out in a cell.
24. The method of paragraph 23, wherein the cell is a eukaryotic cell, such as a mammalian cell (e.g., a human cell, or a rodent cell).
25. The method of paragraph 23 or 24, wherein the cell is within a live organism, such as a mammal (e.g., a human, a non-human mammal, a rodent, or a mouse).
26. The method of any one of paragraphs 23-25, wherein (1) the pegRNA or SVC, (2) the CRISPR/Cas nickase, and/or (3) the nicking sgRNA is/are delivered to the cell via a vector or a non-vector delivery vehicle (such as nanoparticle).
27. The method of paragraph 26, wherein the vector is independently a plasmid, or a viral vector (e.g., an AAV vector, a lentiviral vector, or a retroviral vector).
28. The method of paragraph 27, wherein the AAV vector has a serotype of AAV1, AAV2, AAV3A, AAV3B, AAV4, AAV5, AAV6, AAV7, AAV8, AAV9, AAV10, AAV11, AAV12, AAV13, AAV-DJ, AAV PHP.eB, AAVrh74, or 7m8.
29. A polynucleotide comprising, from 5′ to 3′, (2)-(4) of any one of paragraphs 1-10.
30. A polynucleotide encoding the pegRNA of any one of paragraphs 1-17, the petRNA of any one of paragraphs 1-10, or the polynucleotide of paragraph 29.
31. A vector comprising the polynucleotide of paragraph 30.
32. A cell comprising the polynucleotide of paragraph 30, or the vector of paragraph 31.
33. A pharmaceutical composition comprising the pegRNA, petRNA or SVC of any one of paragraphs 1-17, the polynucleotide of paragraph 29 or 30, the vector of paragraph 31, or the cell of paragraph 32, and a pharmaceutically acceptable diluent or excipient.
34. A kit comprising the pegRNA, petRNA or SVC of any one of paragraphs 1-17, the polynucleotide of paragraph 29 or 30, the vector of paragraph 31, or the cell of paragraph 32, and instructions for inserting a donor DNA sequence at a target DNA sequence.

It should be understood that any one embodiment of the invention described herein, including embodiments described only in the examples or the claims, can be combined with one or more other embodiments unless the combination is expressly disclaimed or improper.

### BRIEF DESCRIPTION OF THE DRAWINGS

FIG. 1A is a schematic (not necessarily to scale) drawing showing a possible (non-binding) working model of an embodiment of the invention. The single prime editing guide RNA (pegRNA) encodes a 2^{nd} primer binding sequence (PBS). Thus the 3′ end of the cDNA can bind the nicking site and initiate a 2^{nd} reverse transcription (RT) reaction.

FIG. 1B is an alternative schematic (not necessarily to scale) drawing showing a possible (non-binding) working model of an embodiment of the invention, e.g., jump prime editing (TJ-PE), which mediates large genomic insertions. TJ-pegRNA: template jump prime editing guide RNA; PBS1: primer binding site 1; RC-PBS2: reverse complement sequence of PBS2.

FIG. 1C shows that a 200 nt insertion was made via the subject single pegRNA-mediated prime editing. In lane 3, 293T cells were transfected with the PE2 prime editing enzyme comprising a Cas nickase fused to a reverse transcriptase, the subject pegRNA with two PBS sequences, and a nicking sgRNA that creates the nick to initiate 2^{nd} strand reverse transcription from the 3′—OH group in the nick. As a control, lane 2 has no PE2 product since only a control nicking sgRNA was provided, and thus 2^{nd} strand reverse transcription cannot proceed in the absence of the 3′-OH at the projected 2^{nd} PBS binding site.

FIG. 1D shows insertion of DNA fragments with PE3 control or TJ-PE at AAVS1 site. HEK293T cells were transfected with PE2, nicking sgRNA, and either TJ-pegRNA (TJ-PE) or control pegRNA (PE3). PCR using primers flanking AAVS1 detected amplicons of 200, 300, and 500-bp insertions with a deletion of 90 bp at the AAVS1 locus. Insertion bands of expected size are denoted with arrows. Ins: insertion, WT: wild-type.

FIG. 1E shows insertion efficiency at AAVS1 locus measured by ddPCR. Results were obtained from three independent experiments, shown as mean±s.d.

FIG. 1F shows the results of verifying accurate insertions using Sanger sequencing of the gel-purified insertion bands.

FIG. 1G confirms precise insertion by TA cloning and Sanger sequencing of 12 individual clones.

FIG. 1H shows insertion of 200 bp determined by deep sequencing in AAVS1 locus.

FIGS. 2A-2C compare the subject method (FIG. 2A) with the published TwinPE method (FIG. 2B) for a short 100 nt insertion. FIG. 2C shows comparable insertion efficiency between the two methods.

FIG. 3A shows successful insertion of a 100-bp DNA inserted at the AAVS1 genomic site, based on SANGER sequencing data of the PCR-amplified insertion site. The data shows that the subject biPE method successfully inserted a 100-bp DNA between the pegRNA and the nicking sgRNA sites, leading to a simultaneous 90-bp deletion (i.e., 100 INS/90 DEL).

FIG. 3B shows the design of the pegRNA and nicking sgRNA transcription units (both transcription driven by the U6 promoter). Note that the RTT template length of 100-500 bp.

FIG. 3C shows that the subject biPE method enables insertion of about 500 bp DNA at the AAVS1 genomic locus. Specifically, 293T cells were transfected with plasmids encoding the elements of the subject biPE system (Cas nickase fused to RT, pegRNA and nicking sgRNA transcription cassettes, etc.). Arrows on the gel image denote insertion bands of predicted size, i.e., about 200 bp, 300 bp, and 500 bp, respectively. WT, wildtype PCR band. MW, molecular weight. *, non-specific band.

FIG. 4A is a schematic (not to scale) illustration of biPE-mediated genomic deletion. In this case, the RTT length is zero (or can be just a few bases linking the two PBS sequences, and the size of the deletion is defined by the predicted nicking sites on the two DNA strands by the pegRNA and the specific nicking sgRNA (87 bp in the illustration).

FIG. 4B shows the design of the pegRNA and nicking sgRNA. Note that there is no RTT sequence between the two PBS sites.

FIG. 4C shows successful deletion of genomic sequence by the subject biPE system. Specifically, 293T cells were transfected with coding sequences for the PE2 enzyme, the pegRNA, and the specific nicking sgRNA (or the control sgRNA that nicks at a position away from the PBS2 binding site). PBS2 binds at the specific nicking sgRNA-created site, but not the control nicking sgRNA-created site, and successful deletion only occurred when the specific nicking sgRNA was provided.

FIG. 5A shows a schematic (not to scale) illustration of positioning the PBS2-associated nicking site upstream of the pegRNA nicking site, and the resulting duplication of the region between the two nicking sites. The duplicated sequence flanks the (optional) RTT sequence (which may or may not exist).

FIG. 5B shows the results of 5′ nicking vs. 3′ nicking using the PBS2-associated specific sgRNA.

FIGS. 6A-6C show TJ-PE mediates insertions at multiple genomic loci. FIG. 6A shows insertion of a 200-bp DNA fragment at HEK3 locus by TJ-PE. HEK293T cells were transfected with PE2, nicking sgRNA, and either pegRNA with a control RC-PBS2 (ctrl-RC-PBS2), or a control nicking sgRNA (ctrl-NK) as controls. The insertion band of predicted size was observed following TJ-PE treatment but not controls (arrow). FIG. 6B shows insertion efficiency at HEK3 measured by ddPCR. FIG. 6C shows insertion of DNA fragments with PE3 control (pegRNA with a control RC-PBS2 sequence) or TJ-PE at PRNP (left) and IDS (right) loci. Insertion efficiency was measured by ddPCR. Results were obtained from three independent experiments, shown as mean±s.d.

FIG. 6D shows insertion of a 200-bp DNA fragment measured by ddPCR at multiple loci in U-2 OS cells. U-2 OS cells transfected with PE plasmid served as control.

FIG. 6E shows insertion of a 200-bp DNA fragment measured by ddPCR at multiple loci in A549 cells. A549 cells transfected with PE plasmid served as control.

FIG. 6F shows insertion efficiency of a 200-bp DNA fragment with various lengths of PBS2 measured by ddPCR. Results were obtained from three independent experiments, shown as mean±s.d.

FIG. 6G compares insertions of GFP fragment to the same sequences containing LoxP at the HEK3 locus. Insertion efficiency quantified by ddPCR.

FIGS. 7A-7F show TJ-PE mediated-GFP reporter and functional gene insertion. FIG. 7A is a diagram of the TLR-MCV1 reporter line. Inserting an 89-bp sequence to replace the 39-bp non-functional sequence results in GFP expression. Indels result in mCherry expression. Del: deletion. In FIG. 7B, PE3 control and TJ-PE were tested in the TLR-MCV1 reporter line, and flow cytometry was used to determine percentage of fluorescent cells. FIG. 7C is a schematic of TJ-pegRNA and targeting strategy for inserting SA-GOI at AAVS1 locus. SA: splice acceptor; GOI: gene of interest. FIG. 7D are bright field and fluorescence images of HEK293T cells 4 days after transfection with PE, TJ-pegRNA, and nicking sgRNA. HEK293T cells transfected with PE plasmid only served as a control (ctrl). Scale bar, 100 μm. FIG. 7E shows efficiency of SA-GFP insertion measured by flow cytometry. Results obtained from three independent experiments were shown as mean±s.d. FIG. 7F shows Agarose gel of PCR amplicons showing SA-GFP and SA-Puro insertion. Puro: puromycin. The insertion bands of expected sizes are indicated with arrow. The nonspecific bands are indicated with asterisk.

FIGS. 8A-8E show in vitro transcribed split circular TJ-petRNA enables large insertion. FIG. 8A shows illustration of split circular TJ-petRNA. The prime editing template RNA (petRNA) sequence carrying an RTT-PBS sequence and an MS2 stem-loop aptamer, and circularized via a permuted group I catalytic intron. Yellow: circularization sequence. FIG. 8B is a schematic model of split circular petRNA function in PE. FIG. 8C shows a urea polyacrylamide gel showing split circular TJ-petRNA after splicing, RNase H, and RNase R digestion. Linear, but not circular, RNA is digested by RNase R. FIG. 8D shows editing efficiency of split circular TJ-petRNA at the AAVS1 locus. Synthesized sgRNAs and in vitro transcribed split circular petRNA were co-transfected with nCas9 and MCP-RT mRNA in 293T cells. FL-pegRNA: in vitro transcribed full-length TJ-pegRNA. HEK293T cells were transfected with PE2, TJ-pegRNA, nicking sgRNA plasmids as control. Results were obtained from three independent experiments, shown as mean±s.d. FIG. 8E is an illustration of the circularization pathway to generate split circular TJ-PE. The circularization sequences are immediately 3′ to the 3′ end of the eventually excised 3′ flank sequence, and are immediately 5′ to the 5′ end of the eventually excised 5′ flank sequence.

FIG. 9A-9I show that TJ-PE rewrites a correction exon in mouse liver. FIG. 9A shows a diagram of Fah splicing before and after correction by TJ-PE. FIG. 9B shows a diagram of the TJ-PE strategy at Fah locus. FIG. 9C shows that TJ-PE treatment rescues body weight after NTBC withdrawal. Body weight ratio is normalized to day 0 of NTBC withdrawal. NC: treated with PBS. FIG. 9D is a schematic of the split-intein dual AAV8 and tail vein injection experiments. Four-week-old tyrosinemia I mice were injected with a total of 2×10¹² vg AAV8. FIG. 9E show representative FAH IHC images. Scale bars, 100 μm. Mice treated with saline were used as negative controls. The lower panel of AAV is a high-magnification view (box with black line). FIG. 9F shows Hematoxylin and eosin (H&E) staining and Fah immunohistochemistry (IHC) staining of mouse liver sections six weeks after NTBC withdrawal. Untreated mice on NTBC served as controls. Scale bar, 100 μm. FIG. 9G shows amplicon sequencing of exon 8 from TJ-PE-treated mouse livers two months after NTBC withdrawal. Editing efficiency results were obtained from three independent experiments, shown as mean±s.d. FIG. 9H shows a schematic of the split-intein dual AAV8 and tail vein injection experiments, and quantification of FAH⁺ hepatocytes by IHC six weeks after AAV injection. Error bars are s.d. (n=4). FIG. 9I shows the results of quantification of FAH⁺ hepatocytes by IHC six weeks after AAV injection. Error bars are s.d. (n=4).

FIG. 10A is a schematic drawing (not to scale) showing that a nicking template jumping prime editor guide RNA (NK-TJ-pegRNA) enables comparable insertion efficiency with TJ-pegRNA. The nicking-TJ-pegRNA (NK-TJ-pegRNA) contains PBS1, RC-PBS2 and an insertion sequence (RTT). Compared to TJ-pegRNA, the PBS1 sequence of NK-TJ-pegRNA first hybridizes to the DNA flap generated by the nicking sgRNA. The newly synthesized PBS2 hybridizes to the second nicked site generated by NK-TJ-pegRNA to initiate the second strand synthesis.

FIG. 10B is an agarose gel image showing insertion bands of expected sizes (200 bp and 300 bp) at AAVS1 locus.

FIG. 10C shows comparable insertion efficiency of nicking TJ PE compared to TJ PE, as quantified by ddPCR.

FIG. 11A is a diagram of pegRNA with a 3′-RNA aptamer.

FIG. 11B shows schematic representations of several structures of the PE-MCP fusion proteins.

FIG. 11C shows insertion efficiency quantified by ddPCR at HEK3 locus. Results were obtained from two independent experiments, shown as mean±s.d.

FIGS. 12A-12C compare insertion efficiency mediated by GRAND and TJ-PE. FIG. 12A is an illustration of TJ-pegRNA and GRAND pegRNA. FIG. 12B shows insertion of 200-bp DNA fragment with TJ-PE or GRAND editing at HEK3, IDS and PRNP loci in HEK293T cells. FIG. 12C shows insertion efficiency of DNA fragment at AAVS1 (500-bp), CCR5 (400-bp), PRNP (400-bp) and IDS (400-bp) loci.

### DETAILED DESCRIPTION OF THE INVENTION

### 1. Overview

The present invention generally relates to genetic engineering, and provides compositions and methods to perform precise genome editing to accurately delete and/or insert large DNA sequences in order to treat a wide range of diseases. In particular, the invention described herein generally relates to methods and compositions to modify/correct genomic sequences (e.g., genomic mutations) that may be associated with diseases or other medical disorders.

The invention described herein differs from the more traditional Prime Editing (PE), including the more recently described Twin Prime Editing (TwinPE) method, in that the present invention can be used to insert much larger polynucleotide sequences at precisely selected location, beyond the capability of these more conventional prime editing methods.

One salient feature of the presently claimed invention is that the prime editing guide RNA, or “pegRNA,” harbors two primer binding sites (PBS1 and PBS2, respectively), whereas the more conventional pegRNA harbors only one PBS, on any given pegRNA. For example, the TwinPE employs two pairs of pegRNA/Cas nickases, with each pegRNA containing one distinct PBS. Due to the unique design feature of the presently described invention, including that of the pegRNA, the invention is capable of inserting much larger donor sequence into selected target DNA sequence.

In one embodiment, the present invention provides a pegRNA with two PBS's capable of supporting the insertion of up to 800 bp or more of donor DNA sequence into a pre-selected target DNA sequence, such as a target DNA sequence inside a human cell.

The data presented herein demonstrates that the subject biPE/TJ-PE system and method can support an efficacious clinical therapy for correcting pathogenic mutations, by replacing/deleting/substituting a large nucleotide sequence with mutation and/or a chromosomal aberration, with a donor sequence, in order to correct the mutation or aberration.

Thus in one aspect, the invention provides a prime editing guide RNA (pegRNA), comprising, from 5′ to 3′: (1) a single guide RNA (sgRNA); (2) a second primer binding sequence (2^{nd} PBS); (3) an optional reverse transcription template (RTT) sequence; and, (4) a first primer binding sequence (1^{st} PBS); wherein: (i) the sgRNA is capable of forming a complex with a CRISPR/Cas nickase and targeting the complex to a target (e.g., target genomic) DNA sequence through base pairing with a targeting strand of the target (genomic) DNA sequence to enable nicking of the non-targeting strand reverse complementary to the targeting strand by the CRISPR/Cas nickase; (ii) the 1^{st} PBS is capable of annealing with the 3′ end of the nicked non-targeting strand created by the CRISPR/Cas nickase, to prime reverse transcription of the RTT (if present) and the 2^{nd} PBS by a reverse transcriptase (RT); optionally, the RT is fused to the CRISPR/Cas nickase; and, (iii) the reverse transcription product of the 2^{nd} PBS is capable of annealing to an anchor sequence on the targeting strand, wherein nicking the targeting strand 3′ to the anchor sequence (e.g., by the CRISPR/Cas nickase and a nicking sgRNA) creates a 3′ end of the targeting strand capable of being extended by the RT to form a second strand cDNA, using the reverse transcribed RTT (if present) and the 1^{st} PBS as template; wherein the reverse complement sequence of the anchor sequence on the non-targeting strand is either upstream (5′) or downstream (3′) of the 1^{st} PBS binding sequence.

In an alternative embodiment, the pegRNA can be replaced with a split variant combination (SVC), wherein the SVC comprises: (a) the sgRNA; and, (b) a prime editing template RNA (petRNA) comprising, from 5′ to 3′, (2)-(4), wherein the petRNA further comprises a linked aptamer (such as MS2) that specifically binds an aptamer binding protein (such as MCP or a functional fragment thereof that binds MS2).

The SVC can be particularly useful when the petRNA component of the SVC can be produced in large quantity using, for example, in vitro transcription. See Example IV. The SVC alternative embodiment enables alternative delivery means, such as non-viral (e.g., RNA-based) delivery of gene editors. Such non-viral delivery possesses numerous advantages, such as easiness of production scaling up, transient expression, lack of detrimental host immune response against heterologous RNA, and minimum off-target effects, etc.

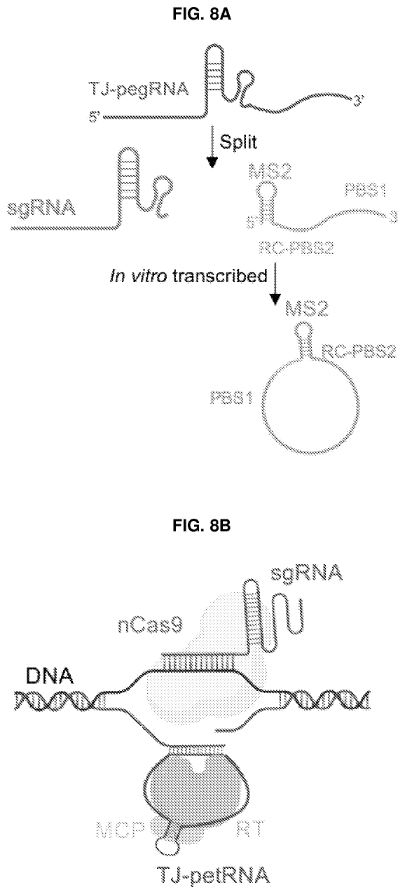

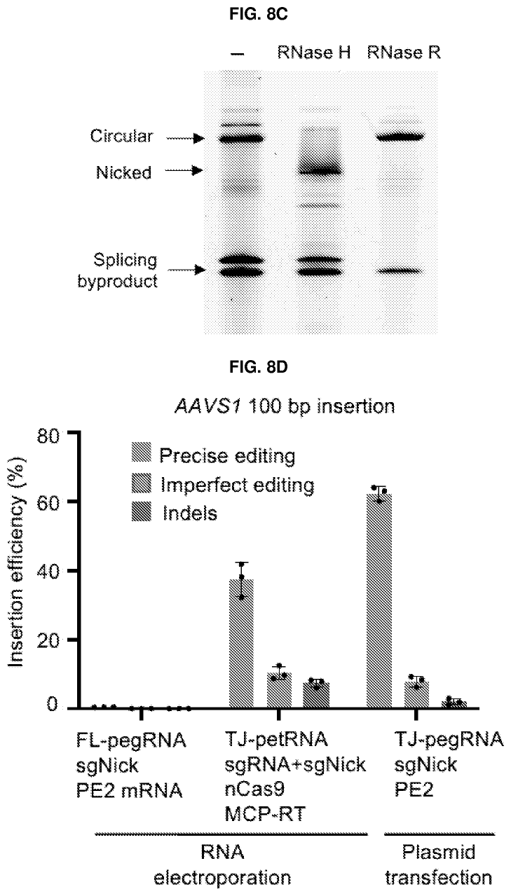

In certain embodiments, the petRNA component of the SVC is a circular RNA, or is produced through an intermediate circular RNA. For example, in some embodiments, the circular petRNA is generated by in vitro transcription to generate a precursor RNA that is circularized post transcriptionally via self-splicing through a permuted group I catalytic intron (see, for example, Wesselhoeft et al., Nature Comm., DOI: 10.1038/s41467-018-05096-6, incorporated herein by reference). Briefly, a group I catalytic intron, such as one of the T4 phage Td gene, can be bisected in such a way to preserve structural elements critical for ribozyme folding. Exon fragment 2 immediately downstream/3′ to the 3′ intron is ligated upstream of (5′ tp) exon fragment 1, and a coding region for the petRNA can be inserted between the exon-exon junction. During splicing, the 3′ hydroxyl group of a guanosine nucleotide engages in a transesterification reaction at the 5′ splice site. The 5′ intron half is excised, and the freed hydroxyl group at the end of the intermediate engages in a second transesterification at the 3′ splice site, resulting in circularization of the intervening region (e.g., the petRNA) and excision of the 3′ intron. See FIG. 8E.

When the SVC embodiment is deployed, a linked aptamer can be included in the petRNA to bring the petRNA to the reverse transcriptase (RT) if the RT is fused to a motif or domain that binds to the aptamer. For example, the MS2 aptamer contains a stem-loop structure from the MS2 bacterial phage genome, which stem-loop structure binds to the MS2 coat protein (MCP).

In some embodiments, the linked aptamer in the petRNA (such as MS2) is immediately 5′ to the 2^{nd} PBS.

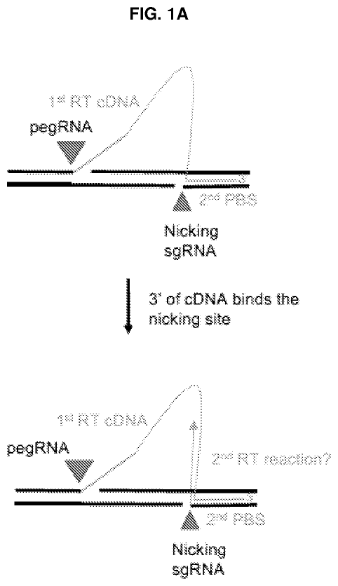

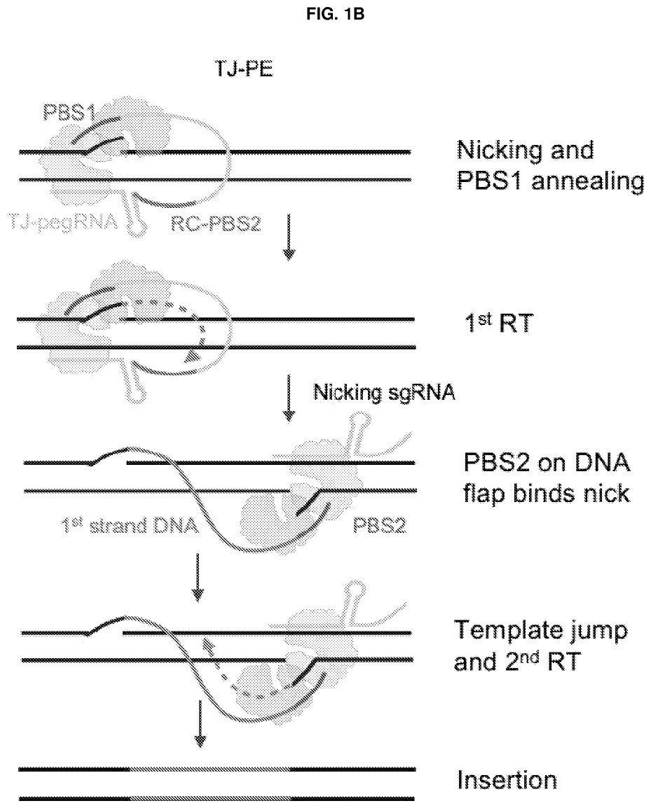

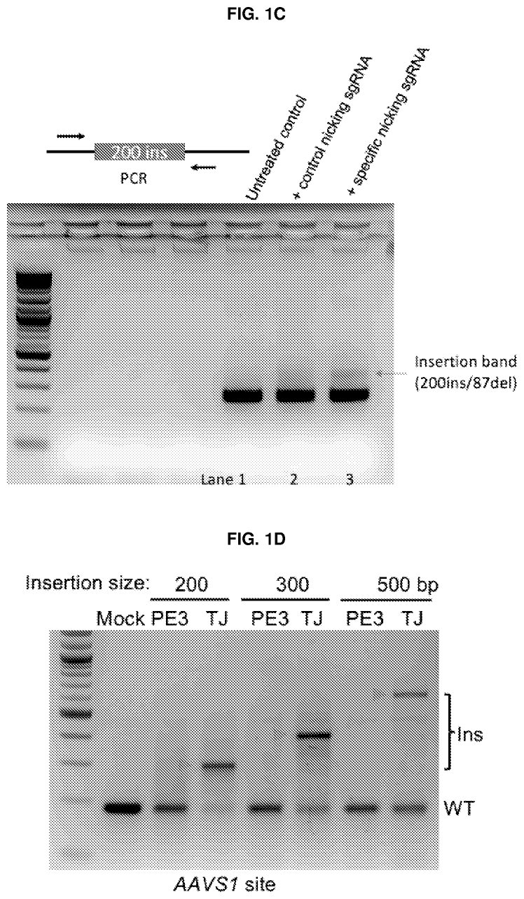

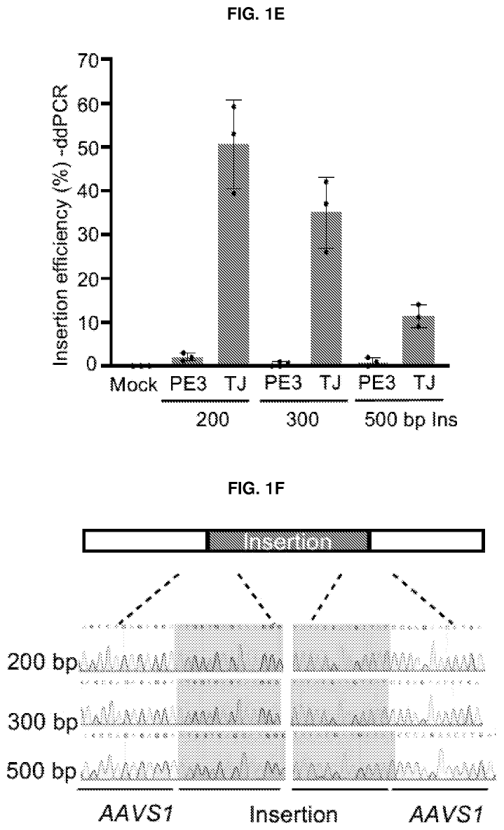

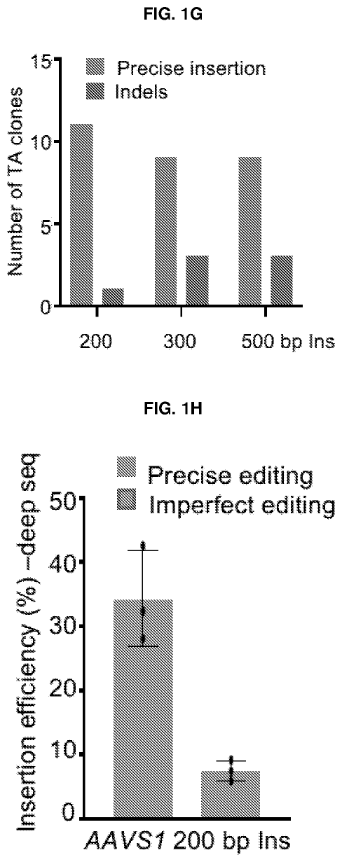

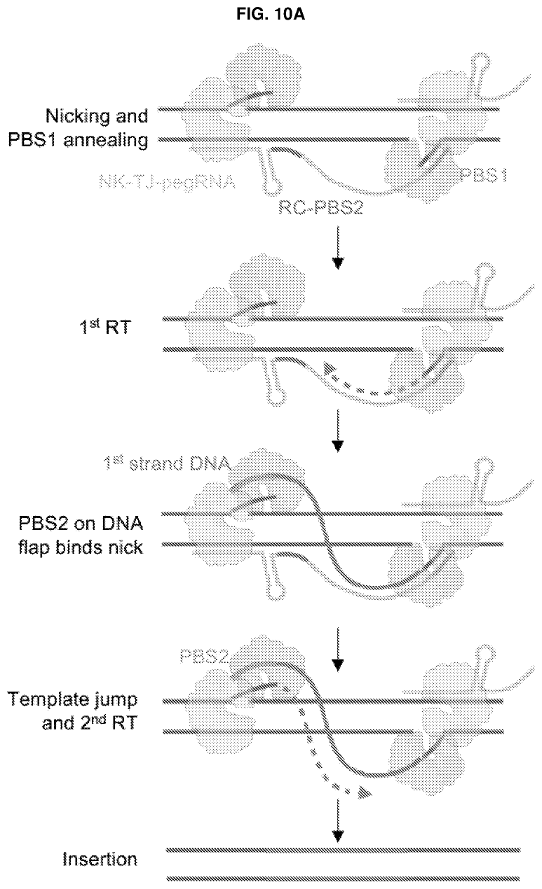

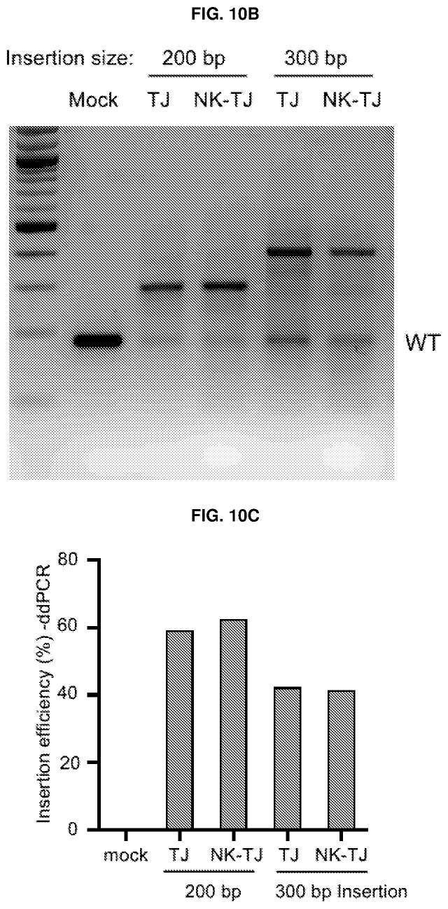

In yet another alternative embodiment, (A) the sgRNA is capable of forming a complex with the CRISPR/Cas nickase and targeting the complex to the target (e.g., target genomic) DNA sequence through base pairing with the targeting strand of the target (genomic) DNA sequence to enable nicking of the non-targeting strand reverse complementary to the targeting strand by the CRISPR/Cas nickase; (B) the 1^{st} PBS is capable of annealing with the 3′ end of the anchor sequence on the targeting strand (resulting from nicking by the CRISPR/Cas nickase and the nicking sgRNA) to prime reverse transcription of the RTT (if present) and the 2^{nd} PBS by the RT; and, (C) the reverse transcription product of the 2^{nd} PBS is capable of annealing to the 3′ end of the nicked non-targeting strand created by the CRISPR/Cas nickase to enable the RT to synthesize a second strand cDNA, using the reverse transcribed RTT (if present) and the 1^{st} PBS as template. See, for example, FIG. 10A (cf. FIG. 1B). It should be noted that this alternative embodiment can be combined with the SVC variation, though FIG. 10A only illustrates the non-SVC embodiment.

In any of the above embodiments, according to this aspect of the invention, the sgRNA portion of the pegRNA or SVC thereof, or petRNA, can be used with a Class 2, Type II CRISPR/Cas nuclease, such as a Cas9-type nuclease, that forms a complex with an sgRNA at or close to the 5′ end of the pegRNA. The sgRNA comprises sequence elements such as a direct repeat (DR) sequence compatible with and forms a complex with the Class 2, Type II (e.g., a Cas9-type) nuclease, and a spacer sequence designed to bind/hybridize/form a double stranded complex with a targeting strand of a target DNA sequence adjacent to a matching/compatible PAM sequence. The Class 2, Type II CRISPR/Cas nuclease, such as a Cas9-type nuclease, has been mutated to become a nickase, such that the nickase has substantially lost the ability to nick the targeting strand, but substantially retains the ability to nick the non-targeting strand of the target DNA sequence, in order to create a 3′—OH group and a 5′-phosphate group.

The very 3′ end of the subject pegRNA, according to this aspect of the invention, comprises a first primer binding sequence (1^{st} PBS), which in one embodiment is capable of annealing with the newly created 3′-end of the nicked non-targeting strand by the Cas-9-type nickase, to prime the reverse transcription of the optional reverse transcription template (or RTT) sequence (if it is present) and the 2^{nd} PBS by a reverse transcriptase (RT). Optionally, the RT can be linked to the Cas9-type nickase, such as through direct fusion of the protein domains, with or without an optional peptide linker (such as a flexible linker based on repeats of G and/or S, including G₄S repeat linker, G₃S repeat linker, G₂S repeat linker, of GS repeat linker, with an overall length of about 1-25 residues, or 5-20 residues, or 10-15 residues) to allow certain degree of flexibility of the linked nickase and RT. In other embodiments, the RT may not be linked to the Cas nickase (see, for example, FIG. 8B). The embodiment may or may not be used in combination with the SVC embodiment of pegRNA.

As used herein, the “2^{nd} PBS” is sometimes referred to as “the reverse complement of the 2^{nd} PBS” or “RC-PBS2.” It should be understood that the RNA sequence element known as the 2^{nd} PBS or PBS2 is not a primer binding sequence, in that it does not actually base-pair with the anchor sequence with a newly generated 3′ end (due to cleavage by the Cas nickase and the nicking guide RNA). Rather, it is the reverse transcription cDNA product of the 2^{nd} PBS that anneals with the anchor sequence (in one embodiment) that promotes second strand cDNA synthesis by the reverse transcriptase.

As used herein, cleavage/nicking by the Cas nickase is not only based on the ability of the sgRNA to guide the Cas complex to the target DNA sequence, but is also predicated on the fact that a suitable protospacer adjacent motif (PAM) sequence compatible with the specific Cas nickase used is adjacent to the target DNA sequence. Thus the term “target DNA sequence” inherently imparts the presence of the PAM adjacent to the target DNA sequence itself. Further, since the nickase that nicks the target strand and the non-target strand may be the same or different, the same or different PAM sequences are present for each specific nickase.

In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

Reverse transcription proceeds to transcribe a first strand cDNA, using the 1^{st} PBS, the optional RTT sequence, and the 2^{nd} PBS of the pegRNA as template. The resulting first strand cDNA comprises a transcribed DNA at the 3′-end with sequence corresponding to and reverse complementary to the 2^{nd} PBS. According to this aspect of the invention, this sequence (the reverse transcription product of the 2^{nd} PBS) at the 3′ end of the first strand cDNA can then serve as a primer to anneal/bind to, for example, an anchor sequence on the targeting strand, wherein nicking the targeting strand (immediately) 3′ to the anchor sequence (e.g., by the Cas9-type CRISPR/Cas nickase and a nicking sgRNA, see below) creates a 3′ end of the targeting strand capable of being extended by the RT to form a second strand cDNA, using the reverse transcribed RTT (if present) and the 1^{st} PBS (PBS1) as template.

The nicking of the targeting strand immediately 3′ to the anchor sequence on the targeting strand can be facilitated by the same Class 2, Type II nuclease (such as the Cas9-type nuclease), when it is complexed with a so-called nicking sgRNA designed to have a compatible DR sequence for the Cas9-type nickase, and a spacer sequence reverse complementary to the non-targeting strand and designed to create a nick immediately 3′ to the anchor sequence by the same Class 2, Type II nuclease (such as the Cas9-type nuclease).

Alternatively, the nicking of the targeting strand immediately 3′ to the anchor sequence on the targeting strand can be facilitated by a different, second nickase, such as another Class 2, Type II nuclease (e.g., a second identical or different Cas9-type nickase not fused to any RT), when it is complexed with a nicking sgRNA designed to have a compatible DR sequence for the second Cas9-type nickase, and a spacer sequence reverse complementary to the targeting strand or non-targeting strand and designed to create a nick immediately 3′ to the anchor sequence by the second Class 2, Type II (such as the Cas9-type) nickase.

Therefore, according to this aspect of the invention, two separate nicks are created on the target DNA, one on the non-targeting strand based on the designed spacer sequence on the pegRNA, and another on the targeting strand based on the designed spacer sequence on the nicking sgRNA. The relative location of the two nicking sites adopt two different configurations.

In one embodiment, the nick on the targeting strand (created by the nicking sgRNA), or strictly speaking, the nucleotide opposite to the nick on the targeting strand, is more downstream or 3′ end to the nick on the non-targeting strand (created by the pegRNA). See FIG. 1A. In this embodiment, in the resulting DNA product, the original DNA sequence between the two nicking sites are replaced by the RTT sequence (if there is an RTT sequence), or is deleted (if there is no RTT sequence, or when RTT sequence has “0 nucleotide”).

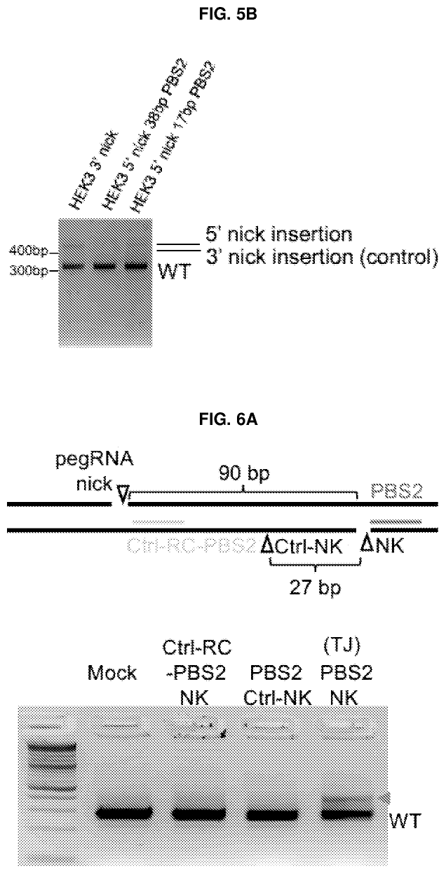

In another embodiment, the nick on the targeting strand (created by the nicking sgRNA), or strictly speaking, the nucleotide opposite to the nick on the targeting strand, is more upstream or 5′ end to the nick on the non-targeting strand (created by the pegRNA). See FIG. 5A. In this embodiment, in the resulting DNA product, the original DNA sequence between the two nicking sites are duplicated and flank the RTT sequence (if there is an RTT sequence), or are simply duplicated (if there is no RTT sequence, or when RTT sequence has “0 nucleotide”).

In certain embodiments, the sgRNA is about 80-120 (e.g., 90-110, or about 100) nucleotides in length. The sgRNA comprises a DR sequence compatible with the Class 2, Type II nuclease (e.g., a Cas9-type nickase), such that the Class 2, Type II (e.g., Cas9-type) nickase can form a complex with the sgRNA. The sgRNA also comprises a spacer sequence designed to hybridize/bind/form a complex with a desired sequence on the targeting strand of the target DNA, adjacent to a PAM sequence compatible with the Class 2, Type II (e.g., Cas9-type) nickase. The spacer sequence is designed such that cleavage or nicking of the non-targeting strand by the Class 2, Type II (e.g., Cas9-type) nickase creates a 3′ end on the non-targeting strand, wherein the 3′-end is substantially reverse complementary in sequence to the 1^{st} PBS in order to prime the reverse transcription from the 3′ end.

In certain embodiments, the spacer sequence on the sgRNA is at least 4-15 nucleotides in length, 8-20 nucleotides in length, or 12-15 nucleotides in length.

In certain embodiments, the optional RTT is absent. In this embodiment, the 1^{st} and the 2^{nd} PBS sequences are directly linked to each other.

In certain embodiments, the optional RTT comprises at least one nucleotide. In certain embodiments, the optional RTT is about 0-900 (e.g., 0-800, 0-850, 5-550, 10-500, 15-400, 20-300, 50-200, 30-60, 40-50, 80-150, 100-120, 0-5, 0, 100, 200, 300, 400, 500, 600, 700, 800, or about 900) nucleotides in length.

In certain embodiments, the 2^{nd} PBS is about 10-20 (e.g., 12-18 or about 15) nucleotides in length. In certain embodiments, the reverse transcription product of the 2^{nd} PBS is substantially reverse complementary in sequence to the anchor sequence, such that it can hybridize with/bind to/form a complex with the anchor sequence.

In certain embodiments, the pegRNA or SVC of the invention further comprises one or more linker(s) or linker sequence(s).

The term “linker,” as used herein, generally refers to a molecule linking two other molecules or moieties. The linker in this context is a nucleotide sequence joining two nucleotide sequences together. For example, in the instant case, the traditional guide RNA or sgRNA can be linked via a linker nucleotide sequence to the RNA extension arm of the subject pegRNA, which may comprise a RTT sequence and two PBS sequences. The nucleotide linker can be 1, 2, 3, 4, 5, 6, 7, 8, 9, 10, 15, 20, 25, 30, 40, 50, 60, 70, 80, 90, or 100 nts in length. Longer or shorter linkers are also contemplated.

The linker may be present between the 1^{st} PBS and the RTT, between the RTT and the 2^{nd} PBS, and/or (in the pegRNA) between the 2^{nd} PBS and the sgRNA. In certain embodiments, the linker in each instance is independently 1, 2, 3, 4, 5, 6, 7, 8, 9, 10, 15, 20, 25, 30, 35, 40, 45, 50, 60, 70, 80, 90, or 100 nucleotides in length. In certain embodiments, each linker is not GC rich (e.g., less than 50%, 40%, or 30% in GC content). In certain embodiments, the linker does not form secondary structure or base pairing with any of the sequence elements of the pegRNA.

Any Class 2, Type II CRISPR/Cas nuclease having guide RNA 5′ to its compatible DR sequence (and thus having 3′ extension to encompass the 1^{st} and 2^{nd} PBS sequences and the RTT sequence) can be used with the pegRNA of the subject invention. Such nucleases can be adapted for use with the pegRNA of the invention by mutating/substantially inactivating one of its endonuclease domains that targets the targeting strand to which the guide RNA binds, but maintaining the endonuclease activity of the other endonuclease domain that targets the non-targeting strand, to create a corresponding CRISPR/Cas nickase.

In certain embodiments, the CRISPR/Cas nickase is a Class 2, Type II Cas effector enzyme. In certain embodiments, the nickase is based on a Cas9, such as SpCas9, SpCas9-HF1, eSpCas9, SaCas9, SaCas9-HF, KKHSaCas9, StCas9, NmCas9, FnCas9, CjCas9, ScCas9, HypaCas9, xCas9, SpRY, SpG, or SauriCas9, which lacks the (HNH) endonuclease activity against the targeting strand.

The same nickase can also be used to create a nick on the targeting strand, when it forms a complex with the nicking sgRNA. For example, the nicking sgRNA may be designed to have a spacer sequence substantially reverse complementary to a sequence on the non-targeting strand, and adjacent to a suitable PAM sequence such that the nicking sgRNA can direct the same nickase to nick the targeting strand, preferably immediately 3′ to the anchor sequence in order to create a free 3′ end to prime the 2^{nd} strand cDNA synthesis once the reverse transcribed 2^{nd} PBS transcript binds to the anchor sequence.

In other words, the CRISPR/Cas nickase lacks endonuclease activity against the non-targeting strand, when forming a complex with the nicking sgRNA to nick the targeting strand (immediately) 3′ to the anchor sequence.

In certain embodiments, the nicking site of the non-targeting strand and the nicking site of the targeting strand are separated by 5, 10, 15, 20, 25, 30, 35, 40, 45, 50, 55, 60, 65, 70, 75, 80, 85, 90, 95, 100, 105, 110, 120, 130, 140, 150, 160, 170, 180, 190, 200, 210, 220, 230, 240, 250, 260, 270, 280, 290, or 300 nucleotides, with the nicking site of the non-targeting strand or the nucleotide directly opposite thereto being either 5′ or 3′ to the nicking site of the targeting strand.

In certain embodiments, the 1^{st} PBS is linked to an RNA element that enhances pegRNA or petRNA stability, and/or improves prime editing efficiency.

RNA elements such as stable pseudoknots at the 3′ end of the pegRNA are well-known in the art to improve prime editing efficiency. Example of such RNA elements include a modified prequeosine₁-1 riboswitch aptamer known as evopreQ1, and the frame-shifting pseudoknot from Moloney murine leukemia virus (MMLV) referred to as “mpknot.” Additional such pseudoknots include those described in Anzalone et al., Nat Methods 13, 453-458 (2016), Houck-Loomis et al., Nature 480, 561-564 (2011); Nahar et al., Chem Commun 54, 2377-2380 (2018); Steckelberg et al., Proc Natl Acad Sci USA 115, 6404-6409 (2018); Cate et al., Science 273, 1678-1685 (1996); and Nelson et al. (Nat Biotechnol. 40(3): 402-410, 2022, all incorporated herein by reference.

In some embodiments, the RNA element comprises a modified/trimmed version of evopreQ1 (tevopreQ1) motif, as described in Nelson et al. (Nat Biotechnol. 40(3): 402-410, 2022, incorporated herein by reference).

In some other embodiments, the RNA element comprises an aptamer such as MS2.

Another aspect of the invention provide a prime editing guide RNA (pegRNA), comprising, from 5′ to 3′: (1) a second primer binding sequence (2^{nd} PBS); (2) an optional reverse transcription template (RTT) sequence; (3) a first primer binding sequence (1^{st} PBS); and, (4) a single guide RNA (sgRNA); wherein: (i) the sgRNA is capable of forming a complex with a CRISPR/Cas nickase and targeting the complex to a target (e.g., a target genomic) DNA sequence through base pairing with a targeting strand of the target (genomic) DNA sequence to enable nicking of the non-targeting strand reverse complementary to the targeting strand by the CRISPR/Cas nickase; (ii) the 1^{st} PBS is capable of annealing with the 3′ end of the nicked non-targeting strand created by the CRISPR/Cas nickase, to prime reverse transcription of the RTT (if present) and the 2^{nd} PBS by a reverse transcriptase (RT); optionally, the RT is fused to the CRISPR/Cas nickase; and, (iii) the reverse transcription product of the 2^{nd} PBS is capable of annealing to an anchor sequence on the targeting strand, wherein nicking the targeting strand 3′ to the anchor sequence (e.g., by the CRISPR/Cas nickase and a nicking sgRNA) creates a 3′ end of the targeting strand capable of being extended by the RT to form a second strand cDNA, using the reverse transcribed RTT (if present) and the 1^{st} PRB as template; wherein the reverse complement sequence of the anchor sequence on the non-targeting strand is either upstream (5′) or downstream (3′) of the 1^{st} PBS binding sequence.

An alternative embodiment of this aspect of the invention is the SVC embodiment as described above, in which the sgRNA and the petRNA are separate polynucleotides.

In a further alternative embodiment, which may or may not be used together with the above SVC embodiment, (A) the sgRNA is capable of forming a complex with the CRISPR/Cas nickase and targeting the complex to the target (e.g., target genomic) DNA sequence through base pairing with the targeting strand of the target (genomic) DNA sequence to enable nicking of the non-targeting strand reverse complementary to the targeting strand by the CRISPR/Cas nickase; (B) the 1^{st} PBS is capable of annealing with the 3′ end of the anchor sequence on the targeting strand (resulting from nicking by the CRISPR/Cas nickase and the nicking sgRNA) to prime reverse transcription of the RTT (if present) and the 2^{nd} PBS by the RT; and, (C) the reverse transcription product of the 2^{nd} PBS is capable of annealing to the 3′ end of the nicked non-targeting strand created by the CRISPR/Cas nickase to enable the RT to synthesize a second strand cDNA, using the reverse transcribed RTT (if present) and the 1^{st} PBS as template;

According to this aspect of the invention, the pegRNA can be used with a Class 2, Type V CRISPR/Cas nuclease, such as a Cpf1-type nuclease, that forms a complex with an sgRNA at or close to the 3′ end of the pegRNA. The sgRNA comprises sequence elements such as a direct repeat (DR) sequence compatible with and forms a complex with the Class 2, Type V (e.g., a Cpf1-type) nuclease, and a spacer sequence designed to bind/hybridize/form a double stranded complex with a targeting strand of a target DNA sequence adjacent to a PAM sequence. The Class 2, Type V CRISPR/Cas nuclease, such as a Cpf1-type nuclease, has been mutated to become a nickase, such that the nickase has substantially lost the ability to nick the targeting strand, but substantially retains the ability to nick the non-targeting strand of the target DNA sequence, in order to create a 3′-OH group and a 5′-phosphate group.

The very 5′ end of the subject pegRNA, according to this aspect of the invention, comprises a second primer binding sequence (2^{nd} PBS), which is capable of annealing with the newly created 3′-end of the nicked non-targeting strand by the Cpf1-type nickase, to prime the reverse transcription of the optional reverse transcription template (or RTT) sequence (if it is present) and the 2^{nd} PBS by a reverse transcriptase (RT). Optionally, the RT can be linked to the Cpf1, such as through direct fusion of the protein domains, with or without an optional peptide linker to allow certain degree of flexibility of the linked nickase and RT.

Reverse transcription proceeds to transcribe a first strand cDNA, using the 1^{st} PBS, the optional RTT sequence, and the 2^{nd} PBS of the pegRNA as template. The resulting first strand cDNA comprises a transcribed DNA at the 3′-end with sequence corresponding to and reverse complementary to the 2^{nd} PBS. According to this aspect of the invention, this sequence (the reverse transcription product of the 2^{nd} PBS) at the 3′ end of the first strand cDNA can then serve as a primer to anneal/bind to, in one embodiment, an anchor sequence on the targeting strand, wherein nicking the targeting strand (immediately) 3′ to the anchor sequence (e.g., by the Cpf1-type CRISPR/Cas nickase and a nicking sgRNA, see below) creates a 3′ end of the targeting strand capable of being extended by the RT to form a second strand cDNA, using the reverse transcribed RTT (if present) and the 1^{st} PBS (PBS1) as template.

The nicking of the targeting strand immediately 3′ to the anchor sequence on the targeting strand can be facilitated by the same Class 2, Type V nuclease (such as the Cpf1-type nuclease), when it is complexed with a so-called nicking sgRNA designed to have a compatible DR sequence for the Cpf1-type nickase, and a spacer sequence reverse complementary to the non-targeting strand and designed to create a nick immediately 3′ to the anchor sequence by the same Class 2, Type V nuclease (such as the Cpf1-type nuclease).

Alternatively, the nicking of the targeting strand immediately 3′ to the anchor sequence on the targeting strand can be facilitated by a different, second nickase, such as another Class 2, Type V nuclease (e.g., a second identical or different Cpf1 not fused to any RT), when it is complexed with a nicking sgRNA designed to have a compatible DR sequence for the second Cpf1, and a spacer sequence reverse complementary to the targeting strand or non-targeting strand and designed to create a nick immediately 3′ to the anchor sequence by the second Class 2, Type V (such as the Cpf1-type) nickase.

Therefore, according to this aspect of the invention, two separate nicks are created on the target DNA, one on the non-targeting strand based on the designed spacer sequence on the pegRNA, and another on the targeting strand based on the designed spacer sequence on the nicking sgRNA. The relative location of the two nicking sites adopt two different configurations.

In one embodiment, the nick on the targeting strand (created by the nicking sgRNA), or strictly speaking, the nucleotide opposite to the nick on the targeting strand, is more downstream or 3′ end to the nick on the non-targeting strand (created by the pegRNA). See FIG. 1A. In this embodiment, in the resulting DNA product, the original DNA sequence between the two nicking sites is replaced by the RTT sequence (if there is an RTT sequence), or is deleted (if there is no RTT sequence, or when RTT sequence has “0 nucleotide”).

In another embodiment, the nick on the targeting strand (created by the nicking sgRNA), or strictly speaking, the nucleotide opposite to the nick on the targeting strand, is more upstream or 5′ end to the nick on the non-targeting strand (created by the pegRNA). See FIG. 5A. In this embodiment, in the resulting DNA product, the original DNA sequence between the two nicking sites is duplicated and flank the RTT sequence (if there is an RTT sequence), or are simply duplicated (if there is no RTT sequence, or when RTT sequence has “0 nucleotide”).

In certain embodiments, the sgRNA is about 80-120 (e.g., 90-110, or about 100) nucleotides in length. The sgRNA comprises a DR sequence compatible with the Class 2, Type V nuclease (e.g., a Cpf1-type nickase), such that the Class 2, Type V (e.g., Cpf1-type) nickase can form a complex with the sgRNA. The sgRNA also comprises a spacer sequence designed to hybridize/bind/form a complex with a desired sequence on the targeting strand of the target DNA, adjacent to a PAM sequence compatible with the Class 2, Type V (e.g., Cpf1-type) nickase. The spacer sequence is designed such that cleavage or nicking of the non-targeting strand by the Class 2, Type V (e.g., Cpf1-type) nickase creates a 3′ end on the non-targeting strand, wherein the 3′-end is substantially reverse complementary in sequence to the 1^{st} PBS in order to prime the reverse transcription from the 3′ end.

In certain embodiments, the spacer sequence on the sgRNA is at least 4-15 nucleotides in length, 8-20 nucleotides in length, or 12-15 nucleotides in length.

In certain embodiments, the optional RTT is absent. In this embodiment, the 1^{st} and the 2^{nd} PBS sequences are directly linked to each other.

In certain embodiments, the optional RTT comprises at least one nucleotide. In certain embodiments, the optional RTT is about 0-900 (e.g., 0-800, 0-850, 5-550, 10-500, 15-400, 20-300, 50-200, 30-60, 40-50, 80-150, 100-120, 0-5, 0, 100, 200, 300, 400, 500, 600, 700, 800, or about 900) nucleotides in length.

In certain embodiments, the 2^{nd} PBS is about 10-20 (e.g., 12-18 or about 15) nucleotides in length. In certain embodiments, the reverse transcription product of the 2^{nd} PBS is substantially reverse complementary in sequence to the anchor sequence, such that it can hybridize with/bind to/form a complex with the anchor sequence.

In certain embodiments, the pegRNA of the invention further comprises one or more linker(s) or linker sequence(s). The linker may be present between the 1^{st} PBS and the RTT, between the RTT and the 2^{nd} PBS, and/or between the 2^{nd} PBS and the sgRNA. In certain embodiments, the linker in each instance is independently 1, 2, 3, 4, 5, 6, 7, 8, 9, 10, 15, 20, 25, 30, 35, 40, 45, 50, 60, 70, 80, 90, or 100 nucleotides in length. In certain embodiments, each linker is not GC rich (e.g., less than 50%, 40%, or 30% in GC content). In certain embodiments, the linker does not form secondary structure or base pairing with any of the sequence elements of the pegRNA.

Any Class 2, Type V CRISPR/Cas nuclease having guide RNA 3′ to its compatible DR sequence (and thus having 5′ extension to encompass the 1^{st} and 2^{nd} PBS sequences and the RTT sequence) can be used with the pegRNA of the subject invention. Such nucleases can be adapted for use with the pegRNA of the invention by mutating/substantially inactivating one of its endonuclease domains that targets the targeting strand to which the guide RNA binds, but maintaining the endonuclease activity of the other endonuclease domain that targets the non-targeting strand, to create a corresponding CRISPR/Cas nickase.

In certain embodiments, the CRISPR/Cas nickase is a Class 2, Type V Cas effector enzyme. In certain embodiments, the nickase is based on a Cas12a/Cpf1, a Cas12b, a Cas12c, a Cas12d, a Cas12e/CasX, a Cas12f/Cas14, a Cas12g, a Cas12h, a Cas12i, a Cas12k, or a V-U, which lacks the endonuclease activity against the targeting strand.

The same nickase can also be used to create a nick on the targeting strand, when it forms a complex with the nicking sgRNA. For example, the nicking sgRNA may be designed to have a spacer sequence substantially reverse complementary to a sequence on the non-targeting strand, and adjacent to a suitable PAM sequence such that the nicking sgRNA can direct the same nickase to nick the targeting strand, preferably immediately 3′ to the anchor sequence in order to create a free 3′ end to prime the 2^{nd} strand cDNA synthesis once the reverse transcribed 2^{nd} PBS transcript binds to the anchor sequence.

In other words, the CRISPR/Cas nickase lacks endonuclease activity against the non-targeting strand, when forming a complex with the nicking sgRNA to nick the targeting strand (immediately) 3′ to the anchor sequence.

In certain embodiments, the nicking site of the non-targeting strand and the nicking site of the targeting strand are separated by 5, 10, 15, 20, 25, 30, 35, 40, 45, 50, 55, 60, 65, 70, 75, 80, 85, 90, 95, 100, 105, 110, 120, 130, 140, 150, 160, 170, 180, 190, 200, 210, 220, 230, 240, 250, 260, 270, 280, 290, or 300 nucleotides, with the nicking site of the non-targeting strand or the nucleotide directly opposite thereto being either 5′ or 3′ to the nicking site of the targeting strand.

In certain embodiments, the RTT sequence comprise or encodes one or more sequences of interest, including (but not limited to) a protein-encoding sequence, a peptide-encoding sequence, or an RNA-encoding sequence.

In certain embodiments, the RTT sequence comprises or encodes a recombinase site, e.g., a Bxb1 recombinase attB (38 bp) and/or attP (50 bp) site, a recombinase site recognized by Hin recombinase, Gin recombinase, Tn3 recombinase, β-six recombinase, CinH recombinase, ParA recombinase, γδ recombinase, ϕC31 recombinase, TP901 recombinase, TG1 recombinase, φBT1 recombinase, R4 recombinase, φRV1 recombinase, φFC1 recombinase, MR11 recombinase, A118 recombinase, U153 recombinase, and gp29 recombinase, Cre recombinase, FLP recombinase, R recombinase, Lambda recombinase, HK101 recombinase, HK022 recombinase, and/or pSAM2 recombinase.

Another aspect of the invention provides a complex, comprising: (1) the pegRNA or SVC of the invention described herein; and, (2) the compatible CRISPR/Cas nickase of the invention.

In certain embodiments, the complex further comprises a target (e.g., a target genomic) DNA sequence, wherein the target (genomic) DNA sequence base pairs with the sgRNA through a targeting strand of the target (genomic) DNA sequence.

In certain embodiments, the complex further comprises (4) a reverse transcribed first strand cDNA reverse complementary in sequence to the 2^{nd} PBS and the RTT sequence (if present); and optionally, (5) a reverse transcribed second strand cDNA reverse complementary in sequence to the first strand cDNA.

Another aspect of the invention provides a method of inserting a donor DNA sequence into/around/proximate to a target (e.g., a target genomic) DNA sequence, the method comprising contacting the target (genomic) DNA sequence with: (1) the pegRNA or SVC, (2) the CRISPR/Cas nickase, and (3) the nicking sgRNA, of the invention described herein, to permit the synthesis of a first strand cDNA and a second strand cDNA based on the RTT sequence of the pegRNA or SVC, through the reverse transcriptase (RT), wherein the RTT sequence encodes the donor DNA sequence.

In certain embodiments, the method is carried out in vitro.

In certain embodiments, method is carried out in a cell.

In certain embodiments, the cell is a eukaryotic cell, such as a mammalian cell (e.g., a human cell, or a rodent cell).

In certain embodiments, the cell is within a live organism, such as a mammal (e.g., a human, a non-human mammal, a rodent, or a mouse).

In certain embodiments, (1) the pegRNA or SVC, (2) the CRISPR/Cas nickase, and/or (3) the nicking sgRNA is/are delivered to the cell via a vector or a non-vector delivery vehicle (such as nanoparticle).

In certain embodiments, the vector is independently a plasmid, or a viral vector (e.g., an AAV vector, a lentiviral vector, or a retroviral vector).

In certain embodiments, the AAV vector has a serotype of AAV1, AAV2, AAV3A, AAV3B, AAV4, AAV5, AAV6, AAV7, AAV8, AAV9, AAV10, AAV11, AAV12, AAV13, AAV-DJ, AAV PHP.eB, AAVrh74, or 7m8.

Another aspect of the invention provides a polynucleotide comprising, from 5′ to 3′, (2) a second primer binding sequence (2^{nd} PBS); (3) an optional reverse transcription template (RTT) sequence; and, (4) a first primer binding sequence (1^{st} PBS); as described herein above.

Another aspect of the invention provides a polynucleotide encoding the pegRNA of the invention, the petRNA of the invention, or the polynucleotide comprising elements (2)-(4) of the pegRNA as described herein above.

Another aspect of the invention provides a vector comprising the polynucleotide of the invention.

Another aspect of the invention provides a cell comprising the polynucleotide of the invention.

Another aspect of the invention provides a pharmaceutical composition comprising the pegRNA, petRNA or SVC, the vector, or the cell of the invention, and a pharmaceutically acceptable diluent or excipient.

Another aspect of the invention provides a kit comprising the pegRNA, petRNA or SVC, the vector, or the cell, and instructions for inserting a donor DNA sequence at a target DNA sequence.

With the general aspects of the invention having been described, more specific aspects of the invention are further elaborated in the sections below.

### 2. pegRNA

The subject pegRNA comprises multiple sequence elements, including 1^{st} and 2^{nd} PBS sequences and the optional RTT sequence, as well as the sgRNA and optional linkers that link any two adjacent sequence elements. The order of the sequence elements may vary, depending on how the pegRNA is to be used with a compatible CRISPR/Cas nickase, specifically, whether the sgRNA portion of the pegRNA will be located at or near the 5′ or 3′ end of the pegRNA.

These sequence elements are described in further details below.

Guide RNA or Single Guide RNA (“gRNA” or “sgRNA”)

As used herein, the terms “guide RNA,” sgRNA and gRNA are used interchangeably, and they all refer to a particular type of guide nucleic acid which is mostly commonly associated with a CRISPR/Cas nuclease, such as a Class 2, Type II (e.g., a Cas9-type) or a Type V (e.g., a Cpf1-type) nuclease. When associated with a compatible Cas such as Cas9 or Cpf1, sgRNA directs the associated Cas protein to a specific target sequence in a DNA molecule that includes reverse complementarity to the spacer sequence of the guide RNA, to enable cleavage or nicking of at least one strand of the target DNA sequence by the Cas protein or nickase.

In a broader sense, this term also includes the equivalent guide nucleic acid molecules that associate with Cas equivalents, homologs, orthologs, or paralogs, whether naturally occurring or non-naturally occurring (e.g., engineered or recombinant), and which otherwise program the Cas equivalent to localize to a specific target nucleotide sequence.

Exemplary Cas protein equivalents may include any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 353(6299), 2016, the contents of which are incorporated herein by reference.

Additional exemplary sequences are and structures of guide RNAs are provided in WO2021/226558A1 (incorporated herein by reference).

In addition, methods for designing appropriate guide RNA sequences are provided herein.

As used herein, the “guide RNA” is one sequence elements of the pegRNA, which includes additional sequence elements for use with the biPE methods and compositions disclosed herein.

The guide RNA of the subject pegRNA may comprise various structural elements that include, but are not limited to: Spacer sequence (the sequence in the guide RNA which binds to the protospacer in the target DNA (a spacer typically has about 20 nts in length); and gRNA core (or gRNA scaffold or backbone sequence, which refers to the sequence within the gRNA that is responsible for Cas binding, and does not include the 20 bp or so spacer/targeting sequence that is used to guide Cas protein to its target DNA).

As used herein, “spacer sequence” refers to the portion of the sgRNA of about 20 nucleotides, which contains a nucleotide sequence that shares the same sequence as the protospacer sequence in the target DNA sequence. The spacer sequence anneals to the reverse complement of the protospacer sequence to form a ssRNA/ssDNA hybrid structure at the target site and a corresponding R loop ssDNA structure of the endogenous DNA strand.

As used herein, “protospacer” refers to the sequence (˜20 bp) in DNA adjacent to the PAM (protospacer adjacent motif) sequence. The protospacer shares the same sequence as the spacer sequence of the guide RNA. The guide RNA anneals to the reverse complement of the protospacer sequence on the target DNA (specifically, one strand thereof, i.e., the “target strand” versus the “non-target strand” of the target DNA sequence). In order for Cas9 to function, it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand.

As used herein, “protospacer adjacent sequence” or “PAM” refers to an approximately 2-6 base pair DNA sequence that is an important targeting component of a Cas nuclease. Typically, the PAM sequence is on either strand, and is downstream in the 5′ to 3′ direction of the Cas cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence. For example, with reference to the canonical SpCas9 amino acid sequence (SEQ ID NO: 18 of WO2021/226558A1, incorporated herein by reference), the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

The term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, and can be packaged into adeno-associated virus (AAV). Further reference may be made to Shah et al., RNA Biology, 10(5): 891-899 (which is incorporated herein by reference).

An “extension arm” as used herein refers to a single strand extension from either the 3′ end or the 5′ end of the sgRNA, which extension arm comprises the 1^{st} and the 2^{nd} primer binding sites (PBS1 and PBS2) and the optional RTT sequence (plus any optional linkers). The RTT and the PBSs form a DNA synthesis template that encodes, via a polymerase (e.g., a reverse transcriptase), a single stranded DNA flap containing the genetic change of interest, which can then be integrated into the endogenous DNA by replacing the corresponding endogenous strand, thereby installing the desired genetic change.

### Reverse Transcription Template (RTT) Sequence

As used herein, the term “Reverse Transcription Template” or RTT sequence refers to the region or portion of the extension arm of a pegRNA that is utilized as a template strand by a polymerase of a prime editor to encode a 3′ single-strand DNA flap that contains the desired edit and which then, through the mechanism of biPE prime editing, replaces and/or adding to the corresponding endogenous strand of DNA at the target site.

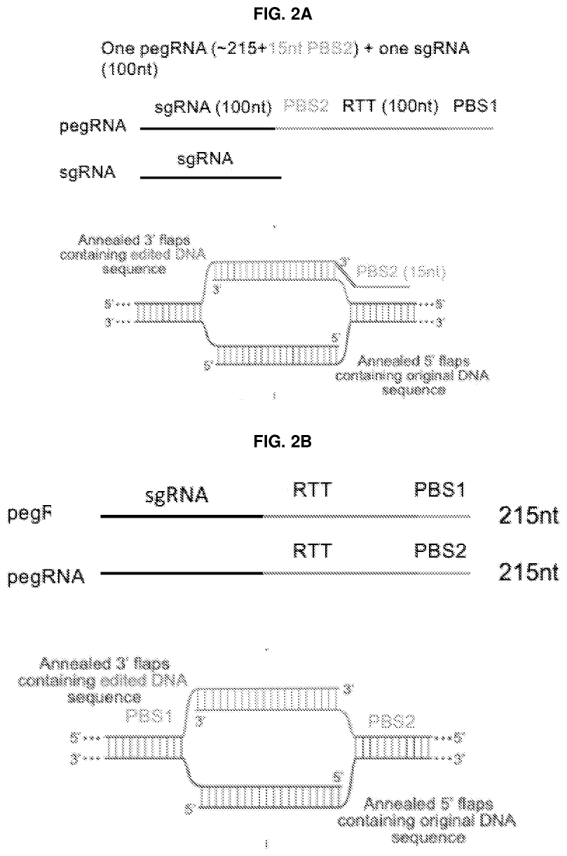

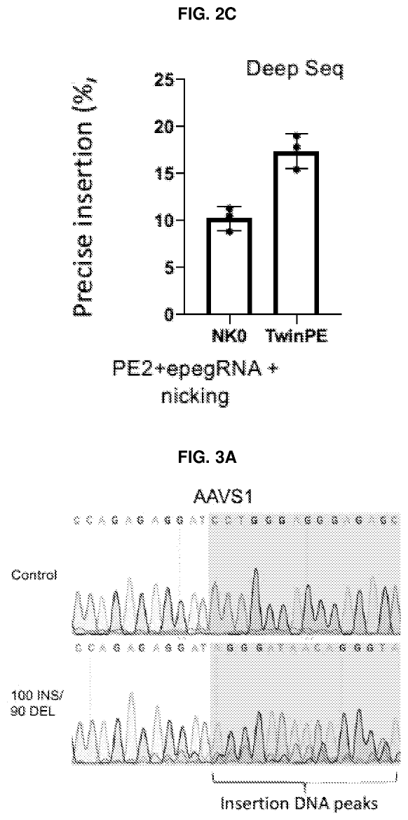

In various embodiments, exemplary RTT is shown in FIGS. 2A, 3B and 5A.

The RTT sequence within the pegRNA is RNA, while its reverse transcription product that is integrated into the DNA target site is DNA, so is the corresponding RTT coding sequence for the pegRNA. Preferably, the RTT sequence excludes the 1^{st} and the 2^{nd} primer binding site (PBS) of the subject pegRNA.

The RTT sequence is flanked by the two PBS of the invention (i.e., the 1^{st} PBS or PBS1, and the 2^{nd} PBS or PBS2).

Reverse transcription using RTT as a template is carried out by a reverse transcriptase (RT), or an RNA-dependent DNA polymerase. Polymerization may terminate in a variety of ways, including, but not limited to (a) reaching a 5′ terminus of the pegRNA (e.g., in the case of the 5′ extension arm for use with the Cpf1-type CRISPR/Cas nuclease, wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA. Either (b) or (c) or both may be used to terminate the transcription downstream from PBS2, when PBS2 is not at the 5′ end of the pegRNA.

The RTT sequence may be the donor sequence to be incorporated into the target DNA site, such as a target genomic location. There is no limit as to what donor sequence may be present in the RTT sequence.

In certain embodiments, the RTT sequence comprises or encodes a “gene of interest” or “GOI,” which refers to a gene or sequence that encodes a biomolecule of interest (e.g., a protein or an RNA molecule).

A protein of interest can include any intracellular protein, membrane protein, or extracellular protein, e.g., a nuclear protein, transcription factor, nuclear membrane transporter, intracellular organelle associated protein, a membrane receptor, a catalytic protein, and enzyme, a therapeutic protein, a membrane protein, a membrane transport protein, a signal transduction protein, or an immunological protein (e.g., an IgG or other antibody protein), etc.

The gene of interest may also encode an RNA molecule, including, but not limited to, messenger RNA (mRNA), transfer RNA (tRNA), ribosomal RNA (rRNA), small nuclear RNA (snRNA), antisense RNA, guide RNA, microRNA (miRNA), small interfering RNA (siRNA), and cell-free RNA (cfRNA).

In certain embodiments, the RTT sequence comprises a recombinase recognition sequence (or “RRS,” “recombinase target sequence,” or “recombinase site”), which refers to a nucleotide sequence target recognized by a recombinase, and which undergoes strand exchange with another DNA molecule having the RRS that results in excision, integration, inversion, or exchange of DNA fragments between the recombinase recognition sequences.

RTT comprising RRS can be used to insert into the target DNA sequence one or more recombinase sites, e.g., at adjacent target sites or non-adjacent target sites (e.g., separate chromosomes).

In certain embodiments, single installed recombinase sites can be used as “landing sites” for a recombinase-mediated reaction between the genomic recombinase site and a second recombinase site within an exogenously supplied nucleic acid molecule, e.g., a plasmid. This enables the targeted integration of a desired nucleic acid molecule.

In other embodiments, where two recombinase sites are inserted in adjacent regions of DNA (e.g., separated by 25-50 bp, 50-100 bp, 100-200 bp, 200-300 bp, 300-400 bp, 400-500 bp, 500-600 bp, 600-700 bp, 700-800 bp, 800-900 bp, 900-1000 bp, 1000-2000 bp, 2000-3000 bp, 3000-4000 bp, 4000-5000 bp, or more), the recombinase sites can be used for recombinase-mediated excision or inversion of the intervening sequence, or for recombinase-mediated cassette exchange with exogenous DNA having the same recombinase sites.

When the two or more recombinase sites are installed on two different chromosomes, translocation of the intervening sequence can occur from a first chromosomal location to the second.

The term “recombinase” refers to a site-specific enzyme that mediates the recombination of DNA between recombinase recognition sequences (RSS), which results in the excision, integration, inversion, or exchange (e.g., translocation) of DNA fragments between the recombinase recognition sequences.

Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, ϕC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange.

Recombinases have numerous applications, including the creation of gene knockouts/knock-ins and gene therapy applications. See, e.g., Brown et al., “Serine recombinases as tools for genome engineering.” Methods 53(4):372-9, 2011; Hirano et al., “Site-specific recombinases as tools for heterologous gene integration.” Appl. Microbiol. Biotechnol. 92(2):227-39, 2011; Chavez and Calos, “Therapeutic applications of the ΦC31 integrase system.” Curr. Gene Ther. 11(5):375-81, 2011; Turan and Bode, “Site-specific recombinases: from tag-and-target- to tag-and-exchange-based genomic modifications.” FASEB J. 25(12):4088-107, 2011; Venken and Bellen, “Genome-wide manipulations of Drosophila melanogaster with transposons, Flp recombinase, and ΦC31 integrase.” Methods Mol. Biol. 859:203-28, 2012; Murphy, “Phage recombinases and their applications.” Adv. Virus Res. 83:367-414, 2012; Zhang et al., “Conditional gene manipulation: Creating a new biological era.” J. Zhejiang Univ. Sci. B. 13(7):511-24, 2012; Karpenshif and Bernstein, “From yeast to mammals: recent advances in genetic control of homologous recombination.” DNA Repair (Amst). 1; 11(10):781-8, 2012; the entire contents of each are hereby incorporated by reference in their entirety.

The recombinases provided herein are not meant to be exclusive examples of recombinases that can be used in embodiments of the invention. The methods and compositions of the invention can be expanded by database searching for new orthogonal recombinases or designing synthetic recombinases with defined DNA specificities (See, e.g., Groth et al., “Phage integrases: biology and applications.” J. Mol. Biol. 335, 667-678, 2004; Gordley et al., “Synthesis of programmable integrases.” Proc. Natl. Acad. Sci. USA. 106, 5053-5058, 2009; the entire contents of each are hereby incorporated by reference in their entirety).

Other examples of recombinases that are useful in the methods and compositions described herein are known to those of skill in the art, and any new recombinase that is discovered or generated is expected to be able to be used in the different embodiments of the invention.

In some embodiments, the catalytic domains of a recombinase are fused to a nuclease-inactivated RNA-programmable nuclease (e.g., dCas9, or a functional fragment thereof), such that the recombinase domain does not comprise a nucleic acid binding domain or is unable to bind to a target nucleic acid (e.g., the recombinase domain is engineered such that it does not have specific DNA binding activity). Recombinases lacking DNA binding activity and methods for engineering such are known, and include those described by Klippel et al., “Isolation and characterization of unusual gin mutants.” EMBO J. 7: 3983-3989, 1988: Burke et al., “Activating mutations of Tn3 resolvase marking interfaces important in recombination catalysis and its regulation. Mol Microbiol. 51: 937-948, 2004; Olorunniji et al., “Synapsis and catalysis by activated Tn3 resolvase mutants.” Nucleic Acids Res. 36: 7181-7191, 2008; Rowland et al., “Regulatory mutations in Sin recombinase support a structure-based model of the synaptosome.” Mol Microbiol. 74: 282-298, 2009; Akopian et al., “Chimeric recombinases with designed DNA sequence recognition.” Proc Natl Acad Sci USA. 100: 8688-8691, 2003; Gordley et al., “Evolution of programmable zinc finger-recombinases with activity in human cells. J Mol Biol. 367: 802-813, 2007; Gordley et al., “Synthesis of programmable integrases.” Proc Natl Acad Sci USA. 106: 5053-5058, 2009; Arnold et al., “Mutants of Tn3 resolvase which do not require accessory binding sites for recombination activity.” EMBO J. 18: 1407-1414, 1999; Gaj et al., “Structure-guided reprogramming of serine recombinase DNA sequence specificity.” Proc Natl Acad Sci USA. 108(2):498-503, 2011; and Proudfoot et al., “Zinc finger recombinases with adaptable DNA sequence specificity.” PLoS One. 6(4):e19537, 2011; the entire contents of each are hereby incorporated by reference.

For example, serine recombinases of the resolvase-invertase group, e.g., Tn3 and 76 resolvases and the Hin and Gin invertases, have modular structures with autonomous catalytic and DNA-binding domains (See, e.g., Grindley et al., “Mechanism of site-specific recombination.” Ann Rev Biochem. 75: 567-605, 2006, the entire contents of which are incorporated by reference).

The catalytic domains of these recombinases are thus amenable to being recombined with nuclease-inactivated RNA-programmable nucleases (e.g., dCas9, or a fragment thereof) as described herein, e.g., following the isolation of “activated” recombinase mutants which do not require any accessory factors (e.g., DNA binding activities) (See, e.g., Klippel et al., “Isolation and characterisation of unusual gin mutants.” EMBO J. 7: 3983-3989, 1988: Burke et al., “Activating mutations of Tn3 resolvase marking interfaces important in recombination catalysis and its regulation. Mol Microbiol. 51: 937-948, 2004; Olorunniji et al., “Synapsis and catalysis by activated Tn3 resolvase mutants.” Nucleic Acids Res. 36: 7181-7191, 2008; Rowland et al., “Regulatory mutations in Sin recombinase support a structure-based model of the synaptosome.” Mol Microbiol. 74: 282-298, 2009; Akopian et al., “Chimeric recombinases with designed DNA sequence recognition.” Proc Natl Acad Sci USA. 100: 8688-8691, 2003).

Additionally, many other natural serine recombinases having an N-terminal catalytic domain and a C-terminal DNA binding domain are known (e.g., phiC31 integrase, TnpX transposase, IS607 transposase), and their catalytic domains can be co-opted to engineer programmable site-specific recombinases as described herein (See, e.g., Smith et al., “Diversity in the serine recombinases.” Mol Microbiol. 44: 299-307, 2002, the entire contents of which are incorporated by reference).

Similarly, the core catalytic domains of tyrosine recombinases (e.g., Cre, λ integrase) are known, and can be similarly co-opted to engineer programmable site-specific recombinases as described herein (See, e.g., Guo et al., “Structure of Cre recombinase complexed with DNA in a site-specific recombination synapse.” Nature. 389:40-46, 1997; Hartung et al., “Cre mutants with altered DNA binding properties.” J Biol Chem 273:22884-22891, 1998; Shaikh et al., “Chimeras of the Flp and Cre recombinases: Tests of the mode of cleavage by Flp and Cre. J Mol Biol. 302:27-48, 2000; Rongrong et al., “Effect of deletion mutation on the recombination activity of Cre recombinase.” Acta Biochim Pol. 52:541-544, 2005; Kilbride et al., “Determinants of product topology in a hybrid Cre-Tn3 resolvase site-specific recombination system.” J Mol Biol. 355:185-195, 2006; Warren et al., “A chimeric cre recombinase with regulated directionality.” Proc Natl Acad Sci USA. 105:18278-18283, 2008; Van Duyne, “Teaching Cre to follow directions.” Proc Natl Acad Sci USA January 6; 106(1):4-5, 2009; Numrych et al., “A comparison of the effects of single-base and triple-base changes in the integrase arm-type binding sites on the site-specific recombination of bacteriophage λ.” Nucleic Acids Res. 18:3953-3959, 1990; Tirumalai et al., “The recognition of core-type DNA sites by λ integrase.” J Mol Biol. 279:513-527, 1998; Aihara et al., “A conformational switch controls the DNA cleavage activity of X integrase.” Mol Cell. 12:187-198, 2003; Biswas et al., “A structural basis for allosteric control of DNA recombination by λ integrase.” Nature 435:1059-1066, 2005; and Warren et al., “Mutations in the amino-terminal domain of λ-integrase have differential effects on integrative and excisive recombination.” Mol Microbiol. 55:1104-1112, 2005; the entire contents of each are incorporated by reference.

### Primer Binding Site

The term “primer binding site” or “PBS” refers to the two nucleotide sequences (PBS1 and PBS2) located on a pegRNA as components of the extension arm (typically the PBS1 and PBS2 flank the optional RTT sequence, on the extension arm) and serve to bind to the primer sequence that is formed after Cas nicking of the non-targeting strand by the prime editor to initiate reverse transcription (PBS1), and to bind to the anchor sequence to prime the 2^{nd} strand cDNA synthesis by the RT (PBS2), respectively. As detailed elsewhere, when the Cas nickase component of a prime editor nicks one strand of the target DNA sequence, a 3′-end ssDNA flap is formed, which serves a primer sequence that anneals to the PBS1 sequence on the pegRNA to prime first strand cDNA reverse transcription.

### Transcription Terminator

In certain embodiments, the pegRNA comprises a transcription terminator to terminate reverse transcription after PBS2. In certain embodiments, the transcription terminatror comprises an impassable RNA secondary structure (e.g., hairpin or stem/loop). In certain embodiments, the transcription terminator comprises a replication termination signal, e.g., a specific nucleotide sequence that blocks or inhibits the polymerase (e.g., RT), or a nucleic acid topological signal, such as, supercoiled DNA or RNA.

### 3. Class 2, Type II, V, and VI CRISPR/Cas Nucleases

In one aspect, the subject pegRNA can be associated/complexed with a suitable or compatible CRISPR/Cas protein (such as nickase), which pegRNA localizes the Cas/nickase to a target DNA sequence that comprises a targeting strand that is reverse complementary to the sgNA or a portion thereof (e.g., the spacer of a sgRNA which anneals to the protospacer of the DNA target).

Any suitable/compatible Cas/nickase may be used in the subject biPE method or system described herein. In certain embodiments, the Cas may be any Class 2 CRISPR-Cas system, including any type II, type V, or type VI CRISPR-Cas enzyme.

Numerous Class 2, Type II Cas such as Cas9-type Cas or Cas9 orthologs are known in the art. See, e.g., Makarova et al., “Classification and Nomenclature of CRISPR-Cas Systems: Where from Here?,” The CRISPR Journal, Vol. 1. No. 5, 2018, the entire contents of which are incorporated herein by reference. The particular CRISPR-Cas nomenclature used in any given instance herein is not limiting in any way.

In certain embodiments, the following type II, type V, and type VI Class 2 CRISPR-Cas enzymes are art-recognized. Each of these enzymes, and/or variants thereof, may be used with the biPE system described herein: Cas9, Cas12a/Cpf1, Cas12b1, Cas12c, Cas12d, Cas12e, Cas13a, Cas13b, Cas13c, and Cas13d.

In certain embodiments, the Cas is a Cas9, such as SpCas9, SpCas9-HF1, eSpCas9, SaCas9, SaCas9-HF, KKHSaCas9, StCas9, NmCas9, FnCas9, CjCas9, ScCas9, HypaCas9, xCas9, SpRY, SpG, or SauriCas9. Their corresponding nickases may lack the (HNH) endonuclease activity against the targeting strand.

In certain embodiments, the CRISPR/Cas nickase is based on a Class 2, Type V Cas effector enzyme (e.g., Cas12a/Cpf1, Cas12b1 (C2c1), Cas12b2, Cas12c (C2c3), Cas12d, Cas12e/CasX, Cas12f/Cas14, Cas12g, Cas12h, Cas12i, Cas12k, or V-U). The nickase may lack endonuclease activity against the targeting strand.

In certain embodiments, the CRISPR/Cas nickase is based on C2c4, C2c8, C2c5, C2c10, C2c9 Cas13a (C2c2), Cas13d, Cas13c (C2c7), Cas13b (C2c6), or Cas13b.

In certain embodiments, a variant, homolog, ortholog, or paralog, whether naturally occurring or non-naturally occurring (e.g., engineered or recombinant), of the above Cas, such as Cas9/Cpf1, which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9/Cpf1 sequence, such as a reference SpCas9 canonical sequence or a reference Cas12a (Cpf1), can also be used in the biPE methods/systems of the invention.

One aspect of the invention utilizes a Class 2, Type II CRISPR/Cas endonuclease modified as nickase, for use with the pegRNA of the invention. Any such endonucleases capable of utilizing a present pegRNA having a guide RNA at/near the 5′ end of the pegRNA and a 3′ end extension that comprises the two PBS sequences and the RTT sequence may be suitable.

A typical such Cas endonuclease is the various Cas9-type endonucleases, or a functional equivalent thereof.

As used herein, the term “Cas9” or “Cas9 nuclease” includes an RNA-guided nuclease comprising a Cas9 domain, or a functional fragment thereof (e.g., a protein comprising an active or inactive DNA cleavage domain of Cas9, and/or the gRNA binding domain of Cas9).

A “Cas9 domain” as used herein, is a protein fragment comprising an active or inactive endonuclease cleavage domain of Cas9 and/or the gRNA binding domain of Cas9.

A “Cas9 protein” may be a full length Cas9 protein. A Cas9 nuclease is also sometimes referred to as a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain natural spacer sequences, which are sequences reverse complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target reverse complementary to the spacer. The target strand not reverse complementary to crRNA is first cut endonucleolytically, then trimmed 3′-5′ exonucleolytically. In nature, DNA-binding and cleavage typically requires protein and both RNAs. However, single guide RNAs (“sgRNA,” or simply “gNRA”) can be engineered to incorporate aspects of both the crRNA and tracrRNA into a single RNA species. See, e.g., Jinek et al., Science 337:816-821, 2012, the entire contents of which are hereby incorporated by reference.

As used herein, “functional equivalent” refers to a second biomolecule that is equivalent in function, but not necessarily equivalent in structure to a first biomolecule. For example, a “Cas9 equivalent” refers to a protein that has the same or substantially the same functions as a particular Cas9 (such as SpCas9 or SaCas9), but not necessarily the same amino acid sequence. In the context of the disclosure, the specification refers throughout to “a protein X, or a functional equivalent thereof.” In this context, a “functional equivalent” of protein X embraces any homolog, paralog, fragment, naturally occurring, engineered, mutated, or synthetic version of protein X which bears an equivalent function.

Cas9 recognizes a short motif in the CRISPR repeat sequences (the PAM or protospacer adjacent motif) to help distinguish self versus non-self sequence. Cas9 nuclease sequences and structures are well known to those of skill in the art (see, e.g., Ferretti et al., Proc. Natl. Acad. Sci. U.S.A. 98:4658-4663, 2001; Deltcheva et al., Nature 471:602-607, 2011; and Jinek et al., Science 337:816-821, 2012, the entire contents of each of which are incorporated herein by reference. Cas9 orthologs have also been described in various species, including, but not limited to, S. pyogenes and S. thermophilus.

Additional suitable Cas9 nucleases and sequences will be apparent to those of skill in the art based on this disclosure, and such Cas9 nucleases and sequences include Cas9 sequences from the organisms and loci disclosed in Chylinski et al., “The tracrRNA and Cas9 families of type II CRISPR-Cas immunity systems,” RNA Biology 10:5, 726-737, 2013, the entire contents of which are incorporated herein by reference.

In some embodiments, a Cas9 nuclease comprises one or more mutations that partially impair or inactivate at least one of the DNA cleavage domains, such as the HNH domain or the RuvC domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science, 337:816-821, 2012; Qi et al., Cell 28; 152(5):1173-1183, 2013, the entire contents of each of which are incorporated herein by reference.

For example, the DNA cleavage domain of Cas9 is known to include two subdomains, the HNH nuclease subdomain and the RuvC1 subdomain. The HNH subdomain cleaves the strand reverse complementary to the gRNA (or the targeting strand), whereas the RuvC1 subdomain cleaves the non-complementary strand (or the non-targeting strand). Mutations within these subdomains can selectively silence one or both subdomain nuclease activity of Cas9. For example, the mutations D10A and H840A completely inactivate the nuclease activity of S. pyogenes Cas9 (Jinek et al., Science 337:816-821, 2012; Qi et al., Cell 28 152(5):1173-1183, 2013).

In some embodiments, proteins comprising functional fragments of Cas9 are provided. For example, in some embodiments, a protein comprises one of two Cas9 domains: (1) the gRNA binding domain of Cas9; or (2) the DNA cleavage domain of Cas9.

In some embodiments, proteins comprising Cas9 or functional fragments thereof are referred to as “Cas9 variants,” or Cas9 for short. A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 18 of WO2021/226558A1, incorporated herein by reference). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes (e.g., conservative or non-conservative changes) compared to wild type Cas9 (e.g., SpCas9 of SEQ ID NO: 18 of WO2021/226558A1). In some embodiments, the Cas9 variant comprises a functional fragment of SEQ ID NO: 18 of WO2021/226558A1 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 18 of WO2021/226558A1). In some embodiments, the functional fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 18 of WO2021/226558A1).

It should be noted that the terms “Cas9” or “Cas9 nuclease” or “Cas9 moiety” or “Cas9 domain” include any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the biPE methods and systems described herein.

Exemplary Cas9 nuclease sequences and structures are well known to those of skill in the art (see, e.g., Ferretti et al., PNAS USA 98:4658-4663, 2001; Deltcheva et al., Nature 471:602-607, 2011; and Jinek et al., Science 337:816-821, 2012, the entire contents of each of which are incorporated herein by reference.

Several specific examples of Cas9 and Cas9 equivalents are provided below. However, these specific examples are not meant to be limiting.

In one embodiment, the Cas9 is a “canonical SpCas9” nuclease from S. pyogenes. Point mutations can be introduced into SpCas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9, or a variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the amino acid and nucleotide sequences of SEQ ID NOs: 18 & 19, respectively, of WO2021/226558A1 (incorporated by reference).

Useable SpCas9 variants include those having at least 80%, at least 85%, at least 90%, at least 95%, or at least 99% sequence identity with a wild type SpCas9 sequence provided above. These variants may include SpCas9 variants containing one or more mutations, including any known mutation reported with the SwissProt Accession No. Q99ZW2 (SEQ ID NO: 18 of WO2021/226558A1) entry, which include:

| SpCas9 mutation (relative to the amino acid | Function/Characteristic (as reported) (see UniProtKB - |
|---|---|
| sequence of the canonical SpCas9 sequence, | Q99ZW2 (CAS9_STRPT1) entry - oncorporated herein |
| SEQ ID NO: 18) | by reference) |
| D10A | Nickase mutant which cleaves the protospacer strand |
| | (but no cleavage of non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | Almost 50% decreased DNA cleavage |
| 312-409 deletion | No nuclease activity |
| E762A | Nickase |
| H840A | Nickase mutant which cleaves the non-protospacer |
| | strand but does not cleave the protospacer strand |
| N854A | Nickase |
| N863A | Nickase |
| H982A | Decreased DNA cleavage |
| D986A | Nickase |
| 1099-1368 deletion | No nuclease activity |
| R133A | Reduced DNA binding |

Other wild type SpCas9 protein or DNA sequences that may be used in the present disclosure include SEQ ID NOs: 20-25 of WO2021/226558A1 (all incorporated by reference).

In other embodiments, the Cas9 protein is a wild type Cas9 ortholog from another bacterial species different from the canonical Cas9 from S. pyogenes. For example, the following Cas9 orthologs described in WO2021/226558A1 can all be used in connection with the biPE constructs described herein: LfCas9 (SEQ ID NO: 26 of WO2021/226558A1), SaCas9 (SEQ ID NO: 27 or 28 of WO2021/226558A1), StCas9 (SEQ ID NO: 29 of WO2021/226558A1), LcCas9 (SEQ ID NO: 30 of WO2021/226558A1), PdCas9 (SEQ ID NO: 31 of WO2021/226558A1), FnCas9 (SEQ ID NO: 32 of WO2021/226558A1), EcCas9 (SEQ ID NO: 33 of WO2021/226558A1), AhCas9 (SEQ ID NO: 34 of WO2021/226558A1), KvCas9 (SEQ ID NO: 35 of WO2021/226558A1), EfCas9 (SEQ ID NO: 36 of WO2021/226558A1), SaCas9 (SEQ ID NO: 37 of WO2021/226558A1), GtCas9 (SEQ ID NO: 38 of WO2021/226558A1), and ScCas9 (SEQ ID NO: 39 of WO2021/226558A1), all incorporated by reference. In addition, any variant Cas9 orthologs having at least 80%, at least 85%, at least 90%, at least 95%, or at least 99% sequence identity to any of the below orthologs may also be used in the methods/system of the invention.

In certain embodiments, the Cas is a protein described as SEQ ID NOs: 58-63 (SaCas9, NmeCas9, CjCas9, GeoCas9, LbaCas12a, and BhCas12b) of WO2021/226558A1 (incorporated by reference), a variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical thereto.

In some embodiments, the Cas is a “Cas9 equivalent”—a broad term that encompasses any Cas9-like protein that serves the same function as Cas9 in the present biPE despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but that do not necessarily have any similarity with regard to amino acid sequence and/or three-dimensional structure. Any Cas9 equivalent that would provide the same or similar function as Cas9, despite that the Cas9 equivalent may be based on a protein that arose through convergent evolution. For instance, if Cas9 refers to a type II enzyme of the CRISPR-Cas system, a Cas9 equivalent can refer to a type V or type VI enzyme of the CRISPR-Cas system.

For example, Cas12e (CasX) is a Cas9 equivalent that reportedly has the same function as Cas9 but which evolved through convergent evolution. Thus, the Cas12e (CasX) protein described in Liu et al., Nature, 2019, Vol. 566: 218-223, is contemplated to be used with the biPE system/method described herein. In addition, any variant or modification of Cas12e (CasX) is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to Cas12e (CasX) or Cas12d (CasY), which have been described in, for example, Burstein et al., Cell Res. 2017 Feb. 21. doi: 10.1038/cr.2017.21, the entire contents of which is hereby incorporated by reference. Using genome-resolved metagenomics, a number of CRISPR/Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-Cas12e and CRISPR-Cas12d, which are among the most compact systems yet discovered. In some embodiments, Cas9 refers to Cas12e, or a variant of Cas12e. In some embodiments, Cas9 refers to a Cas12d, or a variant of Cas12d. It should be appreciated that other RNA-guided DNA binding proteins may be used and are within the scope of this disclosure. Also see Liu et al., Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12e (CasX) or Cas12d (CasY) protein. In some embodiments, the Cas is a naturally-occurring Cas12e (CasX) or Cas12d (CasY) protein. In some embodiments, the Cas comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the Cas includes, without limitation, Cas9 (e.g., nCas9), Cas12e (CasX), Cas12d (CasY), Cas12a (Cpf1), Cas12b1 (C2c1), Cas13a (C2c2), Cas12c (C2c3), Argonaute, and Cas12b1. One example of a nucleic acid programmable DNA-binding protein that has different PAM specificity than Cas9 is Clustered Regularly Interspaced Short Palindromic Repeats from Prevotella and Francisella (i.e., Cas12a (Cpf1)). Similar to Cas9, Cas12a (Cpf1) is also a Class 2 CRISPR effector, but it is a member of type V subgroup of enzymes, rather than the type II subgroup. It has been shown that Cas12a (Cpf1) mediates robust DNA interference with features distinct from Cas9. Cas12a (Cpf1) is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., Cell (165) 2016, p. 949-962; the entire contents of which is hereby incorporated by reference.

In still other embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 18 of WO2021/226558A1).

In various other embodiments, the Cas can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a variant thereof.

Exemplary Cas9 equivalent protein sequences can include the following: AsCas12a (SEQ ID NO: 64 of WO2021/226558A1) or nickase thereof (SEQ ID NO: 65 of WO2021/226558A1), LbCas12a (SEQ ID NO: 66 of WO2021/226558A1), PcCas12a (SEQ ID NO: 67 of WO2021/226558A1), ErCas12a (SEQ ID NO: 68 of WO2021/226558A1), CsCas12a (SEQ ID NO: 69 of WO2021/226558A1), BhCas12b (SEQ ID NO: 70 of WO2021/226558A1), ThCas12b (SEQ ID NO: 71 of WO2021/226558A1), LsCas12b (SEQ ID NO: 72 of WO2021/226558A1), and DtCas12b (SEQ ID NO: 73 of WO2021/226558A1).

The biEP system described herein may also comprise Cas12a (Cpf1) variants that may be used as a Cas nickase protein domain. The Cas12a (Cpf1) protein has a RuvC-like endonuclease domain that is similar to the RuvC domain of Cas9 but does not have a HNH endonuclease domain, and the N-terminal of Cas12a (Cpf1) does not have the alfa-helical recognition lobe of Cas9. It was shown in Zetsche et al., Cell, 163, 759-771, 2015 (which is incorporated herein by reference) that, the RuvC-like domain of Cas12a (Cpf1) is responsible for cleaving both DNA strands and inactivation of the RuvC-like domain inactivates Cas12a (Cpf1) nuclease activity.

Additional Cas9 variants having modified PAM specificity have been described in the art, such as those in Tables 1-3 and SEQ ID NOs: 74-76 and 88 of WO2021/226558A1 (incorporated herein by reference). Such Cas9 variants can also be used in the biPE system/method of the invention.

Any of the above Cas9 protein or variants thereof may be engineered to lack one of the two nuclease catalytic sites to become a nickase. D10A or H840A mutations in wt Cas9 will turn it into a nickase that nicks the targeting or non-targeting strand. Other amino acid substitutions at D10 and H840 positions, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1), can also be made.

The term “Cas9 nickase” or “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, H983A, D986A, or E762A, or a combination thereof. Exemplary Cas9 nickases are described in SEQ ID NOs: 42-49 of WO2021/226558A1 (all incorporated here by reference).

In certain embodiments, the Cas9 nickase can have a mutation in the RuvC nuclease domain and have one of the following amino acid sequences, or a variant thereof having an amino acid sequence that has at least 80%, at least 85%, at least 90%, at least 95%, or at least 99% sequence identity thereto.

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof. See exemplary nickases in SEQ ID NOs: 50-53 of WO2021/226558A1 (incorporated by ref.) In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

In certain embodiments, variants or homologues of Cas9 (e.g., variants of Cas9 from Streptococcus pyogenes (NCBI Reference Sequence: NC_017053.1 (SEQ ID NO: 20 of WO2021/226558A1) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of Cas9 are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto. See SEQ ID NOs: 54-57 of WO2021/226558A1 (incorporated by reference).

Additional Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a Cas9 nickase), or functional fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 18 of WO2021/226558A1).

In some embodiments, the disclosure also may utilize Cas9 fragments that retain their functionality and that are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the biPE prime editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

Equivalent mutations in the Cas9 homologs, orthologs, and paralogs can be made based on sequence comparison.

In certain embodiments, the Cas endonuclease or a nickase thereof is linked to a reverse transcriptase (RT), such as through protein fusion.

The term “reverse transcriptase” or RT describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA which can then be cloned into a vector for further manipulation. Avian myoblastosis virus (AMV) reverse transcriptase was the first widely used RNA-dependent DNA polymerase (Verma, Biochim. Biophys. Acta 473:1, 1977). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger et al., Biochemistry 22:2365-2372 (1983). Another reverse transcriptase which is used extensively in molecular biology is reverse transcriptase originating from Moloney murine leukemia virus (M-MLV). See, e.g., Gerard, DNA 5:271-279 (1986) and Kotewicz et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

Any RT, including wild type RT, functional fragments, mutants, variants, or truncated variants, and the like, can be used. The RT may include wild type polymerases from eukaryotic, prokaryotic, archael, or viral organisms, and/or the polymerases may be modified by genetic engineering, mutagenesis, directed evolution-based processes.

Any wild type reverse transcriptase obtained from any naturally-occurring organism or virus, or obtained from a commercial or non-commercial source, can be used. In addition, the reverse transcriptases usable herein can include any naturally-occurring mutant RT, engineered mutant RT, or other variant RT, including truncated variants that retain function. The RTs may also be engineered to contain specific amino acid substitutions, such as those specifically disclosed herein.

Reverse transcriptases are multi-functional enzymes typically with three enzymatic activities including RNA- and DNA-dependent DNA polymerization activity, and an RNaseH activity that catalyzes the cleavage of RNA in RNA-DNA hybrids. Some mutants of reverse transcriptases have disabled the RNaseH moiety to prevent unintended damage to the mRNA. These enzymes that synthesize complementary DNA (cDNA) using mRNA as a template were first identified in RNA viruses. Subsequently, reverse transcriptases were isolated and purified directly from virus particles, cells or tissues. (e.g., see Kacian et al., 1971, Biochim. Biophys. Acta 46: 365-83; Yang et al., 1972, Biochem. Biophys. Res. Comm. 47: 505-11; Gerard et al., 1975, J. Virol. 15: 785-97; Liu et al., 1977, Arch. Virol. 55187-200; Kato et al., 1984, J. Virol. Methods 9: 325-39; Luke et al., 1990, Biochem. 29: 1764-69 and Le Grice et al., 1991, J. Virol. 65: 7004-07, each of which are incorporated by reference). More recently, mutants and fusion proteins have been created in the quest for improved properties such as thermostability, fidelity and activity. Any of the wild type, variant, and/or mutant forms of reverse transcriptase which are known in the art or which can be made using methods known in the art are contemplated herein.

The reverse transcriptase (RT) gene (or the genetic information contained therein) can be obtained from a number of different sources. For instance, the gene may be obtained from eukaryotic cells which are infected with retrovirus, or from a number of plasmids which contain either a portion of or the entire retrovirus genome. In addition, messenger RNA-like RNA which contains the RT gene can be obtained from retroviruses. Examples of sources for RT include, but are not limited to, Moloney murine leukemia virus (M-MLV or MLVRT); human T-cell leukemia virus type 1 (HTLV-1); bovine leukemia virus (BLV); Rous Sarcoma Virus (RSV); human immunodeficiency virus (HIV); yeast, including Saccharomyces, Neurospora, Drosophila; primates; and rodents. See, for example, Weiss, et al., U.S. Pat. No. 4,663,290 (1987); Gerard, G. R., DNA:271-79 (1986); Kotewicz, M. L., et al., Gene 35:249-58 (1985); Tanese, N., et al., Proc. Natl. Acad. Sci. (USA):4944-48 (1985); Roth, M. J., at al., J. Biol. Chem. 260:9326-35 (1985); Michel, F., et al., Nature 316:641-43 (1985); Akins, R. A., et al., Cell 47:505-16 (1986), EMBO J.4:1267-75 (1985); and Fawcett, D. F., Cell 47:1007-15 (1986) (each of which are incorporated herein by reference in their entireties).

Exemplary RT enzymes include, but are not limited to, M-MLV reverse transcriptase and RSV reverse transcriptase. Enzymes having reverse transcriptase activity are commercially available. In certain embodiments, the reverse transcriptase is provided in trans to the other components of the biPE system. That is, the reverse transcriptase is expressed or otherwise provided as an individual component, i.e., not as a fusion protein with a Cas nickase. In other embodiments, the RT is fused to the nickase via an optional linker.

A person of ordinary skill in the art will recognize that wild type reverse transcriptases, including but not limited to, Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the subject methods and composition described herein.

Exemplary wild type RT enzymes include: MMLV RT (Ref. Seq. AAA66622.1, or SEQ ID NO: 90 of WO2021/226558A1), MMLV wt RT (SEQ ID NO: 700 of WO2021/226558A1), FLV RT (Ref. Seq. NP955579.1, SEQ ID NO: 91 of WO2021/226558A1), HIV-1 RT, Chain A (Ref. Seq. ITL3-A, or SEQ ID NO: 92 of WO2021/226558A1), HIV-1 RT, Chain B (Ref. Seq. ITL3-B, or SEQ ID NO: 93 of WO2021/226558A1), RSV RT (Ref. Seq. ACL14945, or SEQ ID NO: 94 of WO2021/226558A1), CMV RT (Ref. Seq. AGT42196, or SEQ ID NO: 95 of WO2021/226558A1), Klebsiella penumonia RT (Ref. Seq. RFF81513.1, or SEQ ID NO: 96 of WO2021/226558A1), E. coli RT (Ref. Seq. TGH57013, or SEQ ID NO: 97 of WO2021/226558A1), B. subtilis RT (Refd. Seq. QBJ66766, or SEQ ID NO: 98 of WO2021/226558A1), Eubacterium rectale group II intron RT (SEQ ID NO: 99 of WO2021/226558A1), or Geobacillus stearothermophilus group II intron RT (SEQ ID NO: 100 of WO2021/226558A1).

In addition, the invention contemplates the use of reverse transcriptases that are error-prone, i.e., that may be referred to as error-prone reverse transcriptases or reverse transcriptases that do not support high fidelity incorporation of nucleotides during polymerization. During synthesis of the single-strand DNA flap based on the RT template integrated with the pegRNA, the error-prone reverse transcriptase can introduce one or more nucleotides which are mismatched with the RT template sequence, thereby introducing changes to the nucleotide sequence through erroneous polymerization of the single-strand DNA flap. These errors introduced during synthesis of the single strand DNA flap then become integrated into the double strand molecule through hybridization to the corresponding endogenous target strand, removal of the endogenous displaced strand, ligation, and then through one more round of endogenous DNA repair and/or sequencing processes.

In certain embodiments, the reverse transcriptase may be a variant reverse transcriptase. As used herein, a “variant reverse transcriptase” includes any naturally occurring or genetically engineered variant comprising one or more mutations (including singular mutations, inversions, deletions, insertions, and rearrangements) relative to a reference sequences (e.g., a reference wild type sequence). RT naturally have several activities, including an RNA-dependent DNA polymerase activity, ribonuclease H activity, and DNA-dependent DNA polymerase activity. Collectively, these activities enable the enzyme to convert single-stranded RNA into double-stranded cDNA. In retroviruses and retrotransposons, this cDNA can then integrate into the host genome, from which new RNA copies can be made via host-cell transcription. Variant RT's may comprise a mutation which impacts one or more of these activities (either which reduces or increases these activities, or which eliminates these activities all together). In addition, variant RTs may comprise one or more mutations which render the RT more or less stable, less prone to aggregation, and facilitates purification and/or detection, and/or other the modification of properties or characteristics.

One of ordinary skill in the art will recognize that variant reverse transcriptases derived from other reverse transcriptases, including but not limited to Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the subject methods and composition described herein.

One method of preparing variant RTs is by genetic modification (e.g., by modifying the DNA sequence of a wild-type reverse transcriptase). A number of methods are known in the art that permit the random as well as targeted mutation of DNA sequences (see for example, Ausubel et. al. Short Protocols in Molecular Biology (1995) 3^{rd} Ed. John Wiley & Sons, Inc.). In addition, there are a number of commercially available kits for site-directed mutagenesis, including both conventional and PCR-based methods. Examples include the QuikChange Site-Directed Mutagenesis Kits (AGILENT®), the Q5® Site-Directed Mutagenesis Kit (NEW ENGLAND BIOLABS®), and GeneArt™ Site-Directed Mutagenesis System (THERMOFISHER SCIENTIFIC@).

In addition, mutant reverse transcriptases may be generated by insertional mutation or truncation (N-terminal, internal, or C-terminal insertions or truncations) according to methodologies known to one skilled in the art. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Mutations also embrace “gain-of-function” mutations, which is one which confers an abnormal activity on a protein or cell that is otherwise not present in a normal condition.

An example of a method for random mutagenesis is the so-called “error-prone PCR method.” As the name implies, the method amplifies a given sequence under conditions in which the DNA polymerase does not support high fidelity incorporation. Although the conditions encouraging error-prone incorporation for different DNA polymerases vary, one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase.

Also contemplated herein are reverse transcriptase variants that have altered thermostability characteristics. The ability of a reverse transcriptase to withstand high temperatures is an important aspect of cDNA synthesis. Elevated reaction temperatures help denature RNA with strong secondary structures and/or high GC content, allowing reverse transcriptases to read through the sequence. As a result, reverse transcription at higher temperatures enables full-length cDNA synthesis and higher yields, which can lead to an improved generation of the 3′ flap ssDNA as a result of the biPE prime editing process. Wild type M-MLV reverse transcriptase typically has an optimal temperature in the range of 37-48° C.; however, mutations may be introduced that allow for the reverse transcription activity at higher temperatures of over 48° C., including 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., and higher.

The variant reverse transcriptases contemplated herein, including error-prone RTs, thermostable RTs, increase-processivity RTs, can be engineered by various routine strategies, including mutagenesis or evolutionary processes. In some cases, the variants can be produced by introducing a single mutation. In other cases, the variants may require more than one mutation. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

Variant RT enzymes used herein may also include other “RT variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference RT protein, including any wild type RT, or mutant RT, or fragment RT, or other variant of RT disclosed or contemplated herein or known in the art.

In some embodiments, an RT variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT. In some embodiments, the RT variant comprises a fragment of a reference RT, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference RT. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type RT (M-MLV reverse transcriptase) (e.g., SEQ ID NO: 89 of WO2021/226558A1) or to any of the reverse transcriptases of SEQ ID NOs: 90-100 of WO2021/226558A1.

In some embodiments, the disclosure also may utilize RT fragments which retain their functionality and which are fragments of any herein disclosed RT proteins. In some embodiments, the RT fragment is at least 100 amino acids in length. In some embodiments, the fragment is at least 100, 150, 200, 250, 300, 350, 400, 450, 500, 550, or up to 600 or more amino acids in length.

In still other embodiments, the disclosure also may utilize RT variants which are truncated at the N-terminus or the C-terminus, or both, by a certain number of amino acids which results in a truncated variant which still retains sufficient polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end of the protein. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end of the protein. In still other embodiments, the RT truncated variant has a truncation at the N-terminal and the C-terminal end which are the same or different lengths.

For example, a truncated version of M-MLV reverse transcriptase may be used. In this embodiment, the reverse transcriptase contains 4 mutations (D200N, T306K, W313F, T330P; noting that the L603W mutation present in PE2 is no longer present due to the truncation). The DNA sequence encoding this truncated editor is 522 bp smaller than PE2, and therefore makes its potentially useful for applications where delivery of the DNA sequence is challenging due to its size (i.e., adeno-associated virus and lentivirus delivery). This embodiment is referred to as MMLV-RT(trunc) and has the amino acid sequence of SEQ ID NO: 766 of WO2021/226558A1.

In certain embodiments, the Cas endonuclease or a nickase thereof is further linked to a Nuclear localization sequence (NLS). The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in WO/2001/038547, the contents of which are incorporated herein by reference for its disclosure of exemplary nuclear localization sequences. In some embodiments, a NLS comprises the amino acid sequence PKKKRKV (SEQ ID NO: 80) or MDSLLMNRRKFLYQFKNVRWAKGRRETYLC (SEQ ID NO: 82).

In certain embodiments, the NLS comprises any one of the following NLS from WO2021/226558A1 (SEQ ID NOS: 80-91, 85, 92-94, respectively):

| DESCRIPTION | SEQUENCE | SEQ ID NO: |
|---|---|---|
| NLS OF | PKKKRKV | SEQ ID NO: 16 |
| SV40 | | |
| LARGE T- | | |
| AG | | |
| NLS | MKRTADGSEFESPKKKRKV | SEQ ID NO: 124 |
| NLS | MDSLLMNRRKFLYQFKNVRWA | SEQ ID NO: 17 |
| | KGRRETYLC | |
| NLS OF | AVKRPAATKKAGQAKKKKLD | SEQ ID NO: 190 |
| NUCLEO- | | |
| PLASMIN | | |
| NLS OF | MSRPKANPTKLSENAKKLAKE | SEQ ID NO: 191 |
| EGL-13 | VEN | |
| NLS OF C- | PAAKRVKLD | SEQ ID NO: 192 |
| MYC | | |
| NLS OF | KLKIKRPVK | SEQ ID NO: 193 |
| TUS- | | |
| PROTEIN | | |
| NLS OF | VSRKRPRP | SEQ ID NO: 194 |
| POLYOMA | | |
| LARGE T- | | |
| AG | | |
| NLS OF | EGAPPAKRAR | SEQ ID NO: 195 |
| HEPATITIS | | |
| D VIRUS | | |
| ANTIGEN | | |
| NLS OF | PPQPKKKPLDGE | SEQ ID NO: 196 |
| MURINE | | |
| P53 | | |
| NLS OF | SGGSKRTADGSEFEPKKKRKV | SEQ ID NO: 133 |
| PE1 AND | | |
| PE2 | | |

NLSKRPAAIKKAGQAKKK (SEQ ID NO: 136),
PAAKRVKLD (SEQ ID NO: 192),
QRRNELKRSP (SEQ ID NO: 3934).
NQSSNFGPMKGGNFGGTSSGPYGGGGQYFAKPRNQGGY
(SEQ ID NO: 3935).

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof to a reverse transcriptase.

In various embodiments, the biPE prime editors described herein (with RT provided as either a fusion partner or in trans) can include a variant RT comprising one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N in the wild type M-MLV RT (see SEQ ID NO: 89 of WO2021/226558A1) or at a corresponding amino acid position in another wild type RT polypeptide sequence; or P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X in the wild type M-MLV RT (see SEQ ID NO: 89 of WO2021/226558A1) or at a corresponding amino acid position in another wild type RT polypeptide sequence, wherein “X” can be any amino acid.

Some exemplary reverse transcriptases fused to the Cas nickase of the invention are provided as individual proteins according to various embodiments of this disclosure. Exemplary reverse transcriptases include variants with at least 80%, at least 85%, at least 90%, at least 95%, or at least 99% sequence identity to the following wild-type enzymes or partial enzymes: see SEQ ID NOs: 89, 701-716, and 740.

Further possible RT include any publicly-available reverse transcriptase described or disclosed in any of the following U.S. patents (each of which are incorporated by reference in their entireties): U.S. Pat. Nos. 10,202,658; 10,189,831; 10,150,955; 9,932,567; 9,783,791; 9,580,698; 9,534,201; and 9,458,484, and any variant thereof that can be made using known methods for installing mutations, or known methods for evolving proteins. The following references describe reverse transcriptases in art. Each of their disclosures are incorporated herein by reference in their entireties: Herzig et al., J. Virol. 89, 8119-8129 (2015); Mohr et al., Mol. Cell 72, 700-714.e8 (2018); Zhao et al., RNA 24, 183-195 (2018); Zimmerly & Wu, MDNA3-0058-2014 (2015); Ostertag et al., Annual Review of Genetics 35, 501-538 (2001); Perach & Hizi, Virology 259, 176-189 (1999); Lim et al., J. Virol. 80, 8379-8389 (2006); Zhao, Nature Structural & Molecular Biology 23, 558-565 (2016); Griffiths, Genome Biol. 2, REVIEWS1017 (2001); Baranauskas et al., Protein Eng Des Sel 25, 657-668 (2012); Zimmerly et al., Cell 82, 545-554 (1995); Feng et al., Cell 87, 905-916 (1996); Berkhout, Journal of Virology 73, 2365-2375 (1999); Kotewicz et al., Nucleic Acids Res 16, 265-277 (1988); Arezi & Hogrefe, Nucleic Acids Res 37, 473-481 (2009); Blain & Goff, J. Biol. Chem. 268, 23585-23592 (1993); Xiong & Eickbush, EMBO J 9, 3353-3362 (1990); Herschhorn & Hizi, Cell. Mol. Life Sci. 67, 2717-2747 (2010); Taube et al., Biochem. J. 329 (Pt 3), 579-587 (1998); Liu et al., Science 295, 2091-2094 (2002); Luan et al., Cell 72, 595-605 (1993); Nottingham et al., RNA 22, 597-613 (2016); Telesnitsky & Goff, Proc. Natl. Acad. Sci. U.S.A. 90, 1276-1280 (1993); Halvas et al., Journal of Virology 74, 10349-10358 (2000); Nowak et al., Nucleic Acids Res 41, 3874-3887 (2013); Stamos et al., Molecular Cell 68, 926-939.e4 (2017); Das & Georgiadis, Structure 12, 819-829 (2004).; Avidan et al., European Journal of Biochemistry 269, 859-867 (2002); and Gerard et al., Nucleic Acids Res 30, 3118-3129 (2002); Monot et al., PLOS Genetics 9, e1003499 (2013); Mohr et al., RNA 19, 958-970 (2013); and any of the references noted above which relate to reverse transriptases are hereby incorporated by reference in their entireties, if not already stated so.

In certain embodiments, exemplary reverse transcriptases that can be fused to Cas nickase or provided as individual proteins in trans, according to various embodiments of this disclosure are provided below as: SEQ ID NOs: 89 and 106-122 of WO2021/226558A1 (all incorporated herein). Exemplary reverse transcriptases include variants with at least 80%, at least 85%, at least 90%, at least 95% or at least 99% sequence identity to the wild-type enzymes or partial enzymes are also provided.

In certain embodiments, the fusion of a Cas9 nickase and a RT is PE1 fusion, which, as used herein, refers to a fusion protein comprising Cas9 (H840A) and a wild type MMLV RT having the following structure: [NLS]-[Cas9 (H840A)]-[33-residue linker]-[MMLV_RT(wt)]. See SEQ ID NO: 123 of WO2021/226558A1 (incorporated herein by reference), and copied below. (SEQ ID NO: 94)

(SEQ ID NO: 123)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

indicates data missing or illegible when filed

In certain embodiments, the PE1 fusion is in complex with a subject pegRNA to form a PE1 complex. In certain embodiments, the PE1 fusion is in complex with a subject nicking sgRNA that facilitates the nicking of the targeting strand at the 3′ end of the anchor sequence.

In certain embodiments, the fusion of a Cas9 nickase and a RT is PE2 fusion, which, as used herein, refers to a fusion protein comprising Cas9 (H840A) and a wild type MMLV RT having the following structure: [NLS]-[Cas9 (H840A)]-[33-residue linker]-[MMLV_RT(D200N) (T330P) (L603W) (T306K) (W313F)]. See SEQ ID NO: 134 of WO2021/226558A1 (incorporated herein by reference), and copied below.

In certain embodiments, the PE2 fusion is in complex with a subject pegRNA to form a PE2 complex. In certain embodiments, the PE2 fusion is in complex with a subject nicking sgRNA that facilitates the nicking of the targeting strand at the 3′ end of the anchor sequence. (SEQ ID NO: 95)

(SEQ ID NO: 134)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

indicates data missing or illegible when filed

In certain embodiments, the fusion of a Cas9 nickase and a RT is PE-s fusion, which, as used herein, refers to a fusion protein comprising Cas9 (H840A) and a C-terminally truncated RT having the following structure: [NLS]-[Cas9 (H840A)]-[33-residue linker]-[MMLV_RT]. See SEQ ID NO: 765 of WO2021/226558A1 (incorporated herein by reference), and copied below.

In certain embodiments, the PE-s fusion is in complex with a subject pegRNA to form a PE-s complex. In certain embodiments, the PE-s fusion is in complex with a subject nicking sgRNA that facilitates the nicking of the targeting strand at the 3′ end of the anchor sequence. (SEQ ID NO: 96)

(SEQ ID NO: 765)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

indicates data missing or illegible when filed

Additional exemplary biPE prime editors include SEQ ID NOs: 130, 141, 145, 150, 154, 162-164 of WO2021/226558A1 (incorporated by reference).

Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

In certain embodiments, the biPE prime editors described herein may be delivered to cells as two or more fragments which become assembled inside the cell (either by passive assembly, or by active assembly, such as using split intein sequences) into a reconstituted prime editor. In some cases, the self-assembly may be passive whereby the two or more biPE prime editor fragments associate inside the cell covalently or non-covalently to reconstitute the biPE prime editor. In other cases, the self-assembly may be catalyzed by dimerization domains installed on each of the fragments. Examples of dimerization domains are described herein. In still other cases, the self-assembly may be catalyzed by split intein sequences installed on each of the prime editor fragments.

In certain embodiments, the Cas (such as SpCas9 or Cpf1) is split into two fragments at a split site located between residues 1 and 2, or 2 and 3, or 3 and 4, or 4 and 5, or 5 and 6, or 6 and 7, or 7 and 8, or 8 and 9, or 9 and 10, or between any two pair of residues located anywhere between residues 1-10, 10-20, 20-30, 30-40, 40-50, 50-60, 60-70, 70-80, 80-90, 90-100, 100-200, 200-300, 300-400, 400-500, 500-600, 600-700, 700-800, 800-900, 1000-1100, 1100-1200, 1200-1300, or 1300-1368 of wt Cas (such as SEQ ID NO: 18 of WO2021/226558A1).

### 4. Delivery of biPE Prime Editors

In another aspect, the present disclosure provides for the delivery of the subject biPE prime editors in vitro and in vivo using various strategies, including on separate vectors using split inteins and as well as direct delivery strategies of the ribonucleoprotein complex (i.e., the prime editor complexed to the pegRNA and/or the second-site nicking sgRNA) using techniques such as electroporation, use of cationic lipid-mediated formulations, and induced endocytosis methods using receptor ligands fused to the ribonucleotprotein complexes. Any such methods are contemplated herein.

In some aspects, the invention provides methods comprising delivering one or more biPE prime editor-encoding polynucleotides, such as or one or more vectors as described herein encoding one or more components of the biPE prime editing system described herein, one or more transcripts thereof, and/or one or proteins transcribed therefrom, to a host cell.

In some aspects, the invention further provides cells produced by such methods, and organisms (such as animals, plants, or fungi) comprising or produced from such cells.

In some embodiments, a biPE prime editor as described herein in combination with (and optionally complexed with) a guide sequence is delivered to a cell.

Conventional viral and non-viral based gene transfer methods can be used to introduce nucleic acids in mammalian cells or target tissues. Such methods can be used to administer nucleic acids encoding components of a biPE prime editor system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther.2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In various embodiments, the biPE constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split PE fusion protein that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6 (Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan Al, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the pegRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the pegRNA.

In various embodiments, the pegRNAs and the second-site nicking guide RNAs can be expressed from an appropriate promoter, such as a human U6 (hU6) promoter, a mouse U6 (mU6) promoter, or other appropriate promoter. The pegRNAs and the second-site nicking guide RNAs can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

In certain embodiments, the biPE prime editors can be divided at a split site and provided as two halves of a whole/complete prime editor. The two halves can be delivered to cells (e.g., as expressed proteins or on separate expression vectors) and once in contact inside the cell, the two halves form the complete prime editor through the self-splicing action of the inteins on each prime editor half. Split intein sequences can be engineered into each of the halves of the encoded prime editor to facilitate their transplicing inside the cell and the concomitant restoration of the complete, functioning PE.

These split intein-based methods overcome several barriers to in vivo delivery. For example, the DNA encoding prime editors is larger than the rAAV packaging limit, and so requires special solutions. One such solution is formulating the editor fused to split intein pairs that are packaged into two separate rAAV particles that, when co-delivered to a cell, reconstitute the functional editor protein. Several other special considerations to account for the unique features of biPE prime editing are described, including the optimization of second-site nicking targets and properly packaging biPE prime editors into virus vectors, including lentiviruses and rAAV.

In this aspect, the biPE prime editors can be divided at a split site and provided as two halves of a whole/complete prime editor. The two halves can be delivered to cells (e.g., as expressed proteins or on separate expression vectors) and once in contact inside the cell, the two halves form the complete prime editor through the self-splicing action of the inteins on each prime editor half. Split intein sequences can be engineered into each of the halves of the encoded prime editor to facilitate their transplicing inside the cell and the concomitant restoration of the complete, functioning PE.

In some embodiments, the biPE prime editors may be engineered as two half proteins (i.e., a PE N-terminal half and a PE C-terminal half) by “splitting” the whole prime editor as a “split site.” The “split site” refers to the location of insertion of split intein sequences (i.e., the N intein and the C intein) between two adjacent amino acid residues in the prime editor. More specifically, the “split site” refers to the location of dividing the whole prime editor into two separate halves, wherein in each halve is fused at the split site to either the N intein or the C intein motifs. The split site can be at any suitable location in the prime editor fusion protein, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

In some embodiments, the split site is located in the Cas domain. In other embodiments, the split site is located in the RT domain. In other embodiments, the split site is located in a linker that joins the Cas domain and the RT domain.

In various embodiments, split site design requires finding sites to split and insert an N- and C-terminal intein that are both structurally permissive for purposes of packaging the two half prime editor domains into two different AAV genomes. Additionally, intein residues necessary for trans splicing can be incorporated by mutating residues at the N terminus of the C terminal extein or inserting residues that will leave an intein “scar.”

In some embodiments, the split inteins can be used to separately deliver separate portions of a complete PE fusion protein to a cell, which upon expression in a cell, become reconstituted as a complete PE fusion protein through the trans splicing.

In certain embodiments, the biPE prime editors may be delivered by non-viral delivery strategies involving delivery of a biPE prime editor complexed with pegRNA (i.e., a PE ribonucleoprotein complex) by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther.2:291-297 (1995); Behr et al., Bioconjugate Chem. 5:382-389 (1994); Remy et al., Bioconjugate Chem.5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional reference may be made to the following references that discuss approaches for non-viral delivery of ribonucleoprotein complexes, each of which are incorporated herein by reference. See, Chen et al., JBC (2016): jbc-M116; Zuris et al., Nature biotechnology 33.1 (2015): 73; Rouet et al., JMCS 140.21 (2018): 6596-6603.

Another method that may be employed to deliver the subject biPE prime editors and/or pegRNAs to cells in which the biPE prime editing-based genome editing is desired is by employing the use of messenger RNA (mRNA) delivery methods and technologies. Examples of mRNA delivery methods and compositions that may be utilized in the present disclosure including, for example, PCT/US2014/028330, U.S. Pat. No. 8,822,663B2, NZ700688A, ES2740248T3, EP2755693A4, EP2755986A4, WO2014152940A1, EP3450553B1, BR112016030852A2, and EP3362461A1, each of which are incorporated herein by reference in their entireties. Additional disclosure hereby incorporated by reference can be found in Kowalski et al., Mol Therap., 2019; 27(4): 710-728.

In contrast to DNA vector encoding biPE prime editors, the use of RNA as a delivery agent for biPE prime editors has the advantage that the genetic material does not have to enter the nucleus to perform its function. The delivered mRNA may be directly translated in the cytoplasm into the desired protein (e.g., prime editor fusion protein) and nucleic acid products (e.g., pegRNA). However, in order to be more stable (e.g., resist RNA-degrading enzymes in the cytoplasm), it is in some embodiments necessary to stabilize the mRNA to improve delivery efficiency. Certain delivery carriers such as cationic lipids or polymeric delivery carriers can also help protect the transfected mRNA from endogenous RNase enzymes that might otherwise degrade the therapeutic mRNA encoding the desired prime editor fusion proteins. In addition, despite the increased stability of modified mRNA, delivery of mRNA, particularly mRNA encoding full-length protein, to cells in vivo in a manner that allows therapeutic levels of protein production remains a challenge.

With some exceptions, the intracellular delivery of mRNA is generally more challenging than that of small oligonucleotides, and it requires encapsulation into a delivery nanoparticle, in part due to the significantly larger size of mRNA molecules (300-5,000 kDa, ˜1-15 kb) as compared to other types of RNAs (small interfering RNAs [siRNAs], ˜14 kDa; antisense oligonucleotides [ASOs], 4-10 kDa).

mRNA must cross the cell membrane in order to reach the cytoplasm. The cell membrane is a dynamic and formidable barrier to intracellular delivery. It is made up primarily of a lipid bilayer of zwitterionic and negatively charged phospholipids, where the polar heads of the phospholipids point toward the aqueous environment and the hydrophobic tails form a hydrophobic core.

In some embodiments, the mRNA compositions of the disclosure comprise mRNA (encoding a prime editor and/or pegRNA), a transport vehicle, and optionally an agent that facilitates contact with the target cell and subsequent transfection.

In some embodiments, the mRNA can include one or more modifications that confer stability to the mRNA (e.g., compared to the wild-type or native version of the mRNA) and is involved in the associated abnormal expression of the protein. One or more modifications to the wild type that correct the defect may also be included. For example, the nucleic acids of the invention can include modifications of one or both of a 5′ untranslated region or a 3′ untranslated region. Such modifications may include the inclusion of sequences encoding a partial sequence of the cytomegalovirus (CMV) immediate early 1 (IE1) gene, poly A tail, Cap1 structure, or human growth hormone (hGH). In some embodiments, the mRNA is modified to reduce mRNA immunogenicity.

In one embodiment, the biPE prime editor mRNA in the composition of the invention can be formulated in a liposome transfer vehicle to facilitate delivery to target cells. Contemplated transfer vehicles can include one or more cationic lipids, non-cationic lipids, and/or PEG-modified lipids. For example, the transfer vehicle can include at least one of the following cationic lipids: C12-200, DLin-KC2-DMA, DODAP, HGT4003, ICE, HGT5000, or HGT5001. In embodiments, the transfer vehicle comprises cholesterol (chol) and/or PEG modified lipids. In some embodiments, the transfer vehicle comprises DMG-PEG2K. In certain embodiments, the transfer vehicle has the following lipid formulation: C12-200, DOPE, chol, DMG-PEG2K; DODAP, DOPE, cholesterol, DMG-PEG2K; HGT5000, DOPE, chol, DMG-PEG2K, HGT5001, DOPE, chol, one of DMG-PEG2K.

The present disclosure also provides compositions and methods useful for facilitating transfection of target cells with one or more PE-encoding mRNA molecules. For example, the compositions and methods of the present invention contemplate the use of targeting ligands that can increase the affinity of the composition for one or more target cells. In one embodiment, the targeting ligand is apolipoprotein B or apolipoprotein E, and the corresponding target cells express low density lipoprotein receptors and thus promote recognition of the targeting ligand. A vast number of target cells can be preferentially targeted using the methods and compositions of the present disclosure. For example, contemplated target cells include hepatocytes, epithelial cells, hematopoietic cells, epithelial cells, endothelial cells, lung cells, bone cells, stem cells, mesenchymal cells, nerve cells, heart cells, adipocytes, vascular smooth muscle Includes cells, cardiomyocytes, skeletal muscle cells, beta cells, pituitary cells, synovial lining cells, ovarian cells, testis cells, fibroblasts, B cells, T cells, reticulocytes, leukocytes, granulocytes, and tumor cells. However, it is not limited to these.

In some embodiments, the PE-encoding mRNA may optionally have chemical or biological modifications which, for example, improve the stability and/or half-life of such mRNA or which improve or otherwise facilitate protein production. Upon transfection, a natural mRNA in the compositions of the invention may decay with a half-life of between 30 minutes and several days. The mRNAs in the compositions of the disclosure may retain at least some ability to be translated, thereby producing a functional protein or enzyme. Accordingly, the invention provides compositions comprising and methods of administering a stabilized mRNA. In some embodiments, the activity of the mRNA is prolonged over an extended period of time. For example, the activity of the mRNA may be prolonged such that the compositions of the present disclosure are administered to a subject on a semi-weekly or bi-weekly basis, or more preferably on a monthly, bi-monthly, quarterly or an annual basis. The extended or prolonged activity of the mRNA of the present invention is directly related to the quantity of protein or enzyme produced from such mRNA. Similarly, the activity of the compositions of the present disclosure may be further extended or prolonged by modifications made to improve or enhance translation of the mRNA. Furthermore, the quantity of functional protein or enzyme produced by the target cell is a function of the quantity of mRNA delivered to the target cells and the stability of such mRNA. To the extent that the stability of the mRNA of the present invention may be improved or enhanced, the half-life, the activity of the produced protein or enzyme and the dosing frequency of the composition may be further extended.

Accordingly, in some embodiments, the mRNA in the compositions of the disclosure comprise at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present invention, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. The stabilized mRNA molecules provided herein demonstrate longer half-lives relative to their naturally occurring, unmodified counterparts (e.g. the wild-type version of the mRNA). Also contemplated by the terms “modification” and “modified” as such terms related to the mRNA of the present invention are alterations which improve or enhance translation of mRNA nucleic acids, including for example, the inclusion of sequences which function in the initiation of protein translation (e.g., the Kozak consensus sequence). (Kozak, M., Nucleic Acids Res 15 (20): 8125-48 (1987)).

In some embodiments, the mRNAs used in the compositions of the disclosure have undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

Other suitable polynucleotide modifications that may be incorporated into the PE-encoding mRNA used in the compositions of the disclosure include, but are not limited to, 4′-thio-modified bases: 4′-thio-adenosine, 4′-thio-guanosine, 4′-thio-cytidine, 4′-thio-uridine, 4′-thio-5-methyl-cytidine, 4′-thio-pseudouridine, and 4′-thio-2-thiouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof. The term modification also includes, for example, the incorporation of non-nucleotide linkages or modified nucleotides into the mRNA sequences of the present invention (e.g., modifications to one or both of the 3′ and 5′ ends of an mRNA molecule encoding a functional protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

In some embodiments, PE-encoding mRNAs include a 5′ cap structure.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp(5′(A,G(5′)ppp(5′)A and G(5′)ppp(5′)G. Naturally occurring cap structures comprise a 7-methyl guanosine that is linked via a triphosphate bridge to the 5′-end of the first transcribed nucleotide, resulting in a dinucleotide cap of m7G(5′)ppp(5′)N, where N is any nucleoside. In vivo, the cap is added enzymatically. The cap is added in the nucleus and is catalyzed by the enzyme guanylyl transferase. The addition of the cap to the 5′ terminal end of RNA occurs immediately after initiation of transcription. The terminal nucleoside is typically a guanosine, and is in the reverse orientation to all the other nucleotides, i.e., G(5′)ppp(5′)GpNpNp.

Additional cap analogs include, but are not limited to, a chemical structures selected from the group consisting of m⁷GpppG, m⁷GpppA, m⁷GpppC; unmethylated cap analogs (e.g., GpppG); dimethylated cap analog (e.g., m^{2,7}GpppG), trimethylated cap analog (e.g., m^{2,2,7}GpppG), dimethylated symmetrical cap analogs (e.g., m⁷Gpppm⁷G), or anti reverse cap analogs (e.g., ARCA; m^{7,2′}OmeGpppG, m^{7,2′}dGpppG, m^{7,3′}OmeGpppG, m^{7,3′}dGpppG and their tetraphosphate derivatives) (see, e.g., Jemielity, J. et al., “Novel ‘anti-reverse’ cap analogs with superior translational properties”, RNA, 9: 1108-1122 (2003)).

Typically, the presence of a “tail” serves to protect the mRNA from exonuclease degradation. A poly A or poly U tail is thought to stabilize natural messengers and synthetic sense RNA.

Therefore, in certain embodiments a long poly A or poly U tail can be added to an mRNA molecule thus rendering the RNA more stable. Poly A or poly U tails can be added using a variety of art-recognized techniques. For example, long poly A tails can be added to synthetic or in vitro transcribed RNA using poly A polymerase (Yokoe, et al. Nature Biotechnology. 1996; 14: 1252-1256). A transcription vector can also encode long poly A tails. In addition, poly A tails can be added by transcription directly from PCR products. Poly A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)).

Typically, the length of a poly A or poly U tail can be at least about 10, 50, 100, 200, 300, 400 at least 500 nucleotides. In some embodiments, a poly-A tail on the 3′ terminus of mRNA typically includes about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A or poly U tail or may substitute the poly-A or poly U tail.

PE-encoding mRNAs according to the present disclosure may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In embodiments involving mRNA delivery, the ratio of the mRNA encoding the PE fusion protein to the pegRNA may be important for efficient editing. In certain embodiments, the weight ratio of mRNA (encoding the PE fusion protein) to pegRNA is 1:1. In certain other embodiments, the weight ratio of mRNA (encoding the PE fusion protein) to pegRNA is 2:1. In still other embodiments, the weight ratio of mRNA (encoding the PE fusion protein) to pegRNA is 1:2. In still further embodiments, the weight ratio of mRNA (encoding the PE fusion protein) to pegRNA is selected from the group consisting of about 1:1000, 1:900; 1:800; 1:700; 1:600; 1:500; 1:400; 1:300; 1:200; 1:100; 1:90; 1:80; 1:70; 1:60; 1:50; 1:40; 1:30; 1:20; 1:10; and 1:1. In other embodiments, the weight ratio of mRNA (encoding the PE fusion protein) to pegRNA is selected from the group consisting of about 1:1000, 1:900; 800:1; 700:1; 600:1; 500:1; 400:1; 300:1; 200:1; 100:1; 90:1; 80:1; 70:1; 60:1; 50:1; 40:1; 30:1; 20:1; 10:1; and 1:1.

### 5. Pharmaceutical Compositions

Other aspects of the present disclosure relate to pharmaceutical compositions comprising any of the various components of the biPE prime editing system described herein (e.g., including, but not limited to, the Cas nickase optionally fused to the reverse transcriptases (which can be separately delivered in trans), pegRNAs, 2^{nd} specific nicking sgRNAs, and complexes thereof comprising the fusion proteins and pegRNAs, as well as accessory elements, such as second strand nicking components, polynucleotides encoding the same, vectors comprising the polynucleotides, and cells comprising the biPE systems/polynucleotides/vectors thereof.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### 6. Kits, Cells, Vectors, and Delivery

The compositions of the present disclosure may be assembled into kits. In some embodiments, the kit comprises nucleic acid vectors for the expression of the biPE prime editors described herein. In other embodiments, the kit further comprises appropriate guide nucleotide sequences (e.g., pegRNAs and second-site sgRNAs) or nucleic acid vectors for the expression of such guide nucleotide sequences, to target the Cas9 protein or prime editor to the desired target sequence.

The kit described herein may include one or more containers housing components for performing the methods described herein and optionally instructions for use. Any of the kit described herein may further comprise components needed for performing the assay methods. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided.

As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration.

As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively, it may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

Some aspects of this disclosure provide kits comprising a nucleic acid construct comprising a nucleotide sequence encoding the various components of the biPE prime editing systems (e.g., dual prime editing and quadruple prime editing systems) described herein (e.g., including, but not limited to, the napDNAbps, reverse transcriptases, polymerases, fusion proteins (e.g., comprising napDNAbps and reverse transcriptases (or more broadly, polymerases), extended guide RNAs, and complexes comprising fusion proteins and extended guide RNAs, as well as accessory elements, such as second strand nicking components (e.g., second strand nicking gRNA) and 5′ endogenous DNA flap removal endonucleases for helping to drive the biPE prime editing process towards the edited product formation). In some embodiments, the nucleotide sequence(s) comprises a heterologous promoter (or more than a single promoter) that drives expression of the biPE prime editing system components.

Other aspects of this disclosure provide kits comprising one or more nucleic acid constructs encoding the various components of the biPE prime editing systems described herein, e.g., comprising a nucleotide sequence encoding the components of the biPE prime editing system capable of modifying a target DNA sequence. In some embodiments, the nucleotide sequence comprises a heterologous promoter that drives expression of the biPE prime editing system components.

Some aspects of this disclosure provide kits comprising a nucleic acid construct, comprising (a) a nucleotide sequence encoding a Cas9 nickase fused to a reverse transcriptase and (b) a heterologous promoter that drives expression of the sequence of (a).

Cells that may contain any of the compositions described herein include prokaryotic cells and eukaryotic cells. The methods described herein are used to deliver a Cas9 protein or a biPE prime editor into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, rAAV vectors are delivered into human embryonic kidney (HEK) cells (e.g., HEK 293 or HEK 293T cells). In some embodiments, rAAV vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO—IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

Some aspects of the present disclosure relate to using recombinant virus vectors (e.g., adeno-associated virus vectors, adenovirus vectors, or herpes simplex virus vectors) for the delivery of the biPE prime editors or components thereof described herein, e.g., the split Cas9 protein or a split nucleobase biPE prime editors, into a cell. In the case of a split-PE approach, the N-terminal portion of a PE fusion protein and the C-terminal portion of a PE fusion are delivered by separate recombinant virus vectors (e.g., adeno-associated virus vectors, adenovirus vectors, or herpes simplex virus vectors) into the same cell, since the full-length Cas9 protein or biPE prime editors exceeds the packaging limit of various virus vectors, e.g., rAAV (˜4.9 kb).

Thus, in one embodiment, the disclosure contemplates vectors capable of delivering split biPE prime editor fusion proteins, or split components thereof. In some embodiments, a composition for delivering the split Cas9 protein or split prime editor into a cell (e.g., a mammalian cell, a human cell) is provided. In some embodiments, the composition of the present disclosure comprises: (i) a first recombinant adeno-associated virus (rAAV) particle comprising a first nucleotide sequence encoding a N-terminal portion of a Cas9 protein or prime editor fused at its C-terminus to an intein-N; and (ii) a second recombinant adeno-associated virus (rAAV) particle comprising a second nucleotide sequence encoding an intein-C fused to the N-terminus of a C-terminal portion of the Cas9 protein or prime editor. The rAAV particles of the present disclosure comprise a rAAV vector (i.e., a recombinant genome of the rAAV) encapsidated in the viral capsid proteins.

In some embodiments, the rAAV vector comprises: (1) a heterologous nucleic acid region comprising the first or second nucleotide sequence encoding the N-terminal portion or C-terminal portion of a split Cas9 protein or a split biPE prime editor in any form as described herein, (2) one or more nucleotide sequences comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of a cell. In some embodiments, viral sequences that facilitate integration comprise Inverted Terminal Repeat (ITR) sequences. In some embodiments, the first or second nucleotide sequence encoding the N-terminal portion or C-terminal portion of a split Cas9 protein or a split biPE prime editor is flanked on each side by an ITR sequence. In some embodiments, the nucleic acid vector further comprises a region encoding an AAV Rep protein as described herein, either contained within the region flanked by ITRs or outside the region. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV6.

Thus, in some embodiments, the rAAV particles disclosed herein comprise at least one rAAV2 particle, rAAV6 particle, rAAV8 particle, rPHP.B particle, rPHP.eB particle, or rAAV9 particle, or a variant thereof. In particular embodiments, the disclosed rAAV particles are rPHP.B particles, rPHP.eB particles, rAAV9 particles.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc.2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the rAAV vector of the present disclosure comprises one or more regulatory elements to control the expression of the heterologous nucleic acid region (e.g., promoters, transcriptional terminators, and/or other regulatory elements).

In some embodiments, the first and/or second nucleotide sequence is operably linked to one or more (e.g., 1, 2, 3, 4, 5, or more) transcriptional terminators. Non-limiting examples of transcriptional terminators that may be used in accordance with the present disclosure include transcription terminators of the bovine growth hormone gene (bGH), human growth hormone gene (hGH), SV40, CW3, ϕ, or combinations thereof. The efficiencies of several transcriptional terminators have been tested to determine their respective effects in the expression level of the split Cas9 protein or the split biPE prime editor. In some embodiments, the transcriptional terminator used in the present disclosure is a bGH transcriptional terminator.

In some embodiments, the rAAV vector further comprises a Woodchuck Hepatitis Virus Posttranscriptional Regulatory Element (WPRE). In certain embodiments, the WPRE is a truncated WPRE sequence, such as “W3.” In some embodiments, the WPRE is inserted 5′ of the transcriptional terminator. Such sequences, when transcribed, create a tertiary structure which enhances expression, in particular, from viral vectors.

In some embodiments, the vectors used herein may encode the PE fusion proteins, or any of the components thereof (e.g., Cas nickase-RT, linkers, or polymerases). In addition, the vectors used herein may encode the pegRNAs, and/or the accessory sgRNA for second strand nicking. The vectors may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoters that may be used in the prime editor vectors may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphs1 promoter, OG-2 promoter, SP-B promoter, SYN1 promoter, and WASP promoter.

In some embodiments, the prime editor vectors (e.g., including any vectors encoding the prime editor fusion protein and/or the pegRNAs, and/or the accessory second strand nicking gRNAs) may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the prime editor vectors (e.g., including any vectors encoding the prime editor fusion protein and/or the pegRNAs, and/or the accessory second strand nicking gRNAs) may comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphs1 promoter, OG-2 promoter, SP-B promoter, SYN1 promoter, and WASP promoter.

In some embodiments, the nucleotide sequence encoding the pegRNA (or any guide RNAs used in connection with biPE prime editing) may be operably linked to at least one transcriptional or translational control sequence.

In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, HI and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human HI promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA.

In some embodiments, the nucleotide sequence encoding the guide RNA may be located on the same vector comprising the nucleotide sequence encoding the PE fusion protein. In some embodiments, expression of the guide RNA and of the PE fusion protein may be driven by their corresponding promoters. In some embodiments, expression of the guide RNA may be driven by the same promoter that drives expression of the PE fusion protein. In some embodiments, the guide RNA and the PE fusion protein transcript may be contained within a single transcript. For example, the guide RNA may be within an untranslated region (UTR) of the Cas9 protein transcript. In some embodiments, the guide RNA may be within the 5′ UTR of the PE fusion protein transcript. In other embodiments, the guide RNA may be within the 3′ UTR of the PE fusion protein transcript. In some embodiments, the intracellular half-life of the PE fusion protein transcript may be reduced by containing the guide RNA within its 3′ UTR and thereby shortening the length of its 3′ UTR. In additional embodiments, the guide RNA may be within an intron of the PE fusion protein transcript. In some embodiments, suitable splice sites may be added at the intron within which the guide RNA is located such that the guide RNA is properly spliced out of the transcript. In some embodiments, expression of the Cas9 protein and the guide RNA in close proximity on the same vector may facilitate more efficient formation of the CRISPR complex.

The biPE prime editor vector system may comprise one vector, or two vectors, or three vectors, or four vectors, or five vector, or more. In some embodiments, the vector system may comprise one single vector, which encodes both the PE fusion protein and pegRNA. In other embodiments, the vector system may comprise two vectors, wherein one vector encodes the PE fusion protein and the other encodes the pegRNA. In additional embodiments, the vector system may comprise three vectors, wherein the third vector encodes the second strand nicking gRNA used in the herein methods.

In some embodiments, the composition comprising the rAAV particle (in any form contemplated herein) further comprises a pharmaceutically acceptable carrier. In some embodiments, the composition is formulated in appropriate pharmaceutical vehicles for administration to human or animal subjects.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The terms such as “excipient”, “carrier”, “pharmaceutically acceptable carrier” or the like are used interchangeably herein.

### 7. Delivery Methods

In some aspects, the invention provides methods comprising delivering one or more polynucleotides encoding the various components of the biPE prime editors described herein, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell.

Exemplary delivery strategies are described herein elsewhere, which include vector-based strategies, PE ribonucleoprotein complex delivery, and delivery of PE by mRNA methods.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

Exemplary methods of delivery of nucleic acids include lipofection, nucleofection, electroporation, stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g. in vitro or ex vivo administration) or target tissues (e.g. in vivo administration). Delivery may be achieved through the use of RNP complexes.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther.2:291-297 (1995); Behr et al., Bioconjugate Chem. 5:382-389 (1994); Remy et al., Bioconjugate Chem.5:647-654 (1994); Gao et al., Gene Therapy 2:710-722 (1995); Ahmad et al., Cancer Res.52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In other embodiments, the method of delivery and vector provided herein is an RNP complex. RNP delivery of fusion proteins markedly increases the DNA specificity of base editing. RNP delivery of fusion proteins leads to decoupling of on- and off-target DNA editing. RNP delivery ablates off-target editing at non-repetitive sites while maintaining on-target editing comparable to plasmid delivery, and greatly reduces off-target DNA editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein.

Additional methods for the delivery of nucleic acids to cells are known to those skilled in the art. See, for example, US 2003/0087817, incorporated herein by reference.

Other aspects of the present disclosure provide methods of delivering the biPE prime editor constructs into a cell to form a complete and functional prime editor within a cell. For example, in some embodiments, a cell is contacted with a composition described herein (e.g., compositions comprising nucleotide sequences encoding the split Cas9 or the split prime editor or AAV particles containing nucleic acid vectors comprising such nucleotide sequences). In some embodiments, the contacting results in the delivery of such nucleotide sequences into a cell, wherein the N-terminal portion of the Cas9 protein or the prime editor and the C-terminal portion of the Cas9 protein or the prime editor are expressed in the cell and are joined to form a complete Cas9 protein or a complete prime editor.

It should be appreciated that any rAAV particle, nucleic acid molecule or composition provided herein may be introduced into the cell in any suitable way, either stably or transiently. In some embodiments, the disclosed proteins may be transfected into the cell. In some embodiments, the cell may be transduced or transfected with a nucleic acid molecule. For example, a cell may be transduced (e.g., with a virus encoding a split protein), or transfected (e.g., with a plasmid encoding a split protein) with a nucleic acid molecule that encodes a split protein, or an rAAV particle containing a viral genome encoding one or more nucleic acid molecules. Such transduction may be a stable or transient transduction. In some embodiments, cells expressing a split protein or containing a split protein may be transduced or transfected with one or more guide RNA sequences, for example in delivery of a split Cas9 (e.g., nCas9) protein. In some embodiments, a plasmid expressing a split protein may be introduced into cells through electroporation, transient (e.g., lipofection) and stable genome integration (e.g., piggybac) and viral transduction or other methods known to those of skill in the art.

In certain embodiments, the compositions provided herein comprise a lipid and/or polymer. In certain embodiments, the lipid and/or polymer is cationic. The preparation of such lipid particles is well known. See, e.g. U.S. Pat. Nos. 4,880,635; 4,906,477; 4,911,928; 4,917,951; 4,920,016; 4,921,757; and 9,737,604, each of which is incorporated herein by reference.

The guide RNA sequence may be 15-100 nucleotides in length and comprise a sequence of at least 10, at least 15, or at least 20 contiguous nucleotides that is reverse complementary to a target nucleotide sequence. The guide RNA may comprise a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is reverse complementary to a target nucleotide sequence. The guide RNA may be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the target nucleotide sequence is a DNA sequence in a genome, e.g. a eukaryotic genome. In certain embodiments, the target nucleotide sequence is in a mammalian (e.g. a human) genome.

The compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., a carrier or vehicle.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example I Insertion of Large Donor DNA Sequences Using biDirectional Prime Editing

This example demonstrates that the subject bi-directional prime edit method and system can be used to insert donor DNA sequences (e.g., >200 bp to >500 bp) that are much larger than previously reported traditional prime editing (PE) methods, including TwinPE.

Specifically, human embryonic kidney (HEK293T) cells and HEK293T-TLR cells were transfected, using Lipofectamine 3000 reagent (Invitrogen), by vectors encoding a biPE prime editor comprising a Cas9 nickase fused to an MMLV reverse transcriptase (RT), a subject pegRNA having two PBS sites flanking a donor sequence in the RTT sequence, and a PBS2-associated nicking sgRNA.

The pegRNA was designed to target the AAVS1 genomic locus by containing a spacer sequence in its sgRNA portion specific for the AAVS1 target sequence. The donor sequence within the RTT sequence had various lengths, such as about 200 bp and 500 bp (see SEQ ID NO: 1 below).

Three days post transfection, genomic DNA was isolated from the transfected HEK293T cells, and was PCR-amplified using a pair of primers specific for the insertion site at the AAVS1 genomic locus (see SEQ ID NOs: 2 and 3).

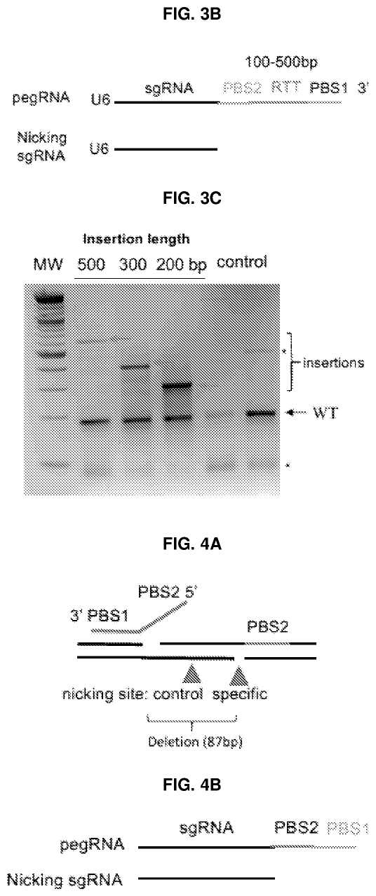

The amplified sequence was analyzed by sequencing, as well as by TIDE (Tracking of Indels by Decomposition) analysis. FIG. 3A shows that the AAVS1 target DNA sequence was successfully inserted by the designed donor sequence. FIG. 3C also shows the successful insertion of 200 bp, 300 bp, and 500 bp donor DNA sequences based on gel electrophoresis analysis.

An earlier similar experiment also showed that a 200 bp donor DNA sequence was successfully inserted by the subject biPE method. See FIG. 1C. FIG. 2C shows that the efficiency of the biPE method is comparable to that of the TwinPE method.

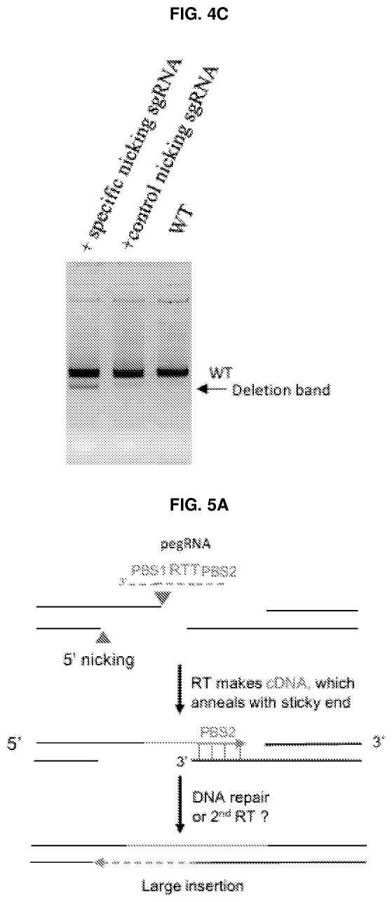

The same method was also used to delete a genomic DNA sequence at a target DNA sequence, according to a scheme illustrated in FIG. 4A, where the optional RTT sequence was missing. See the DNA band with a shorter length in FIG. 4C.

In yet another example, the PBS2 binding anchor sequence was chosen to be more upstream to the PBS1 binding sequence (FIG. 5A), and the so-called 5′ nicking biPE product is bigger because of the duplication of the region between the two nicking sites flanking the donor sequence in the end product. See FIG. 5B.

Detailed experimental steps and conditions used in these experiments are provided below for illustrative purpose only, and are by no means limiting.

### Cell Culture and Transfection

Human embryonic kidney (HEK293T) cells (from ATCC) and HEK293T-TLR cells were maintained in Dulbecco's Modified Eagle's Medium (DMEM, Corning) supplemented with 10% fetal bovine serum (FBS, Gibco) and 1% Penicillin/Streptomycin (Gibco). Cells were seeded at 70% confluence in 12-well cell culture plate one day before transfection. The plasmids containing the coding sequences for the PE (Cas nickase fused to reverse transcriptase), biPE pegRNA, and the PBS2-associated nicking sgRNA were transfected with Lipofectamine 3000 reagent (Invitrogen).

pegRNA Design and Clone

Plasmids expressing pegRNAs were constructed by Gibson assembly using BsaI-digested acceptor plasmid (Addgene #132777) as vector. The sequence of the pegRNA containing 500 bp RTT insertion sequence, for insertion at the AAVS1 genomic locus, is provided below:

AAVS1 +500 bp pegRNA:
(SEQ ID NO: 1)
GATGGAGCCAGAGAGGATCCGTTTTAGAGCTAGAAATAGCAAGTTAAAATAAGGCTAGTCCGTTATCA
ACTTGAAAAAGTGGGACCGAGTCGGTCCGCAGCTCAGGTTCTGGGATAACTTCGTATAATGTATGCTA
TACGAAGTTATAACAATCCCCCAACTGAGAGAACTCAAAGGTTACCCCAGTTGGGGCACATTACCCTG
TTATCCCTACCTCCTTGAAGTCGATGCCCTTCAGCTCGATGCGGTTCACCAGGGTGTCGCCCTCGAAC
TTCACCTCGGCGCGGGTCTTGTAGTTGCCGTCGTCCTTGAAGAAGATGGTGCGCTCCTGGACGTAGCC
TTCGGGCATGGCGGACTTGAAGAAGTCGTGCTGCTTCATGTGGTCGGGGTAGCGGCTGAAGCACTGCA
CGCCGTAGGTCAGGGTGGTCACGAGGGTGGGCCAGGGCACGGGCAGCTTGCCGGTGGTGCAGATGAAC
TTCAGGGTCAGCTTGCCGTAGGTGGCATCGCCCTCGCCCTCGCCGGACACGCTGAACTTGTGGCCGTT
TACGTCGCCGTCCAGCTCGACCAGGATGGGCACCACCCCGGTGAACAGCTCCTCGCCCTTGCTCACCA
TTCCTCTCTGGCTCTCTCTCTCTTGACGCGGTTCTATCTAGTTACGCGTTAAACCAACTAGAAATTTT
TTT

### Genomic DNA Extraction, PCR Amplification and Digestion

To extract genomic DNA, HEK293T cells (3 days post transfection) were washed with PBS, pelleted, and lysed with 50 μL of Quick extraction buffer (Epicenter). The genomic DNA was then incubated with appropriate PCR primers in a thermocycler for PCR amplification (65° C. 15 min, and 98° C. 5 min).

PureLink Genomic DNA Mini Kit (Thermo Fisher) was used to extract genomic DNA from two different liver lobes (˜10 mg each) per mouse. The genomic DNA was amplified similarly as described above.

AAVS1 primers for PCR:
(SEQ ID NO: 2)
CCAGGATCAGTGAAACGCAC
&
(SEQ ID NO: 3)
CTTGCCAGAACCTCTAAGGT

### Tracking of Indels by Decomposition (TIDE) Analysis

The sequences around the two cut sites of the target locus were amplified using Phusion Flash PCR Master Mix (Thermo Fisher). Sanger sequencing was performed to sequence the purified PCR products, and the trace sequences were analyzed using TIDE software (tide.nki.nl). The alignment window of left boundary was set at 10-bp.

### Example II Insertion of Large Donor DNA Sequences Using Template-Jumping (TJ) Prime Editing Using a Single pegRNA—GFP Expression in Cells

Examples II-IV further demonstrate the use of the subject biPE, also referred to hereinafter as “template-jumping (TJ) PE” approach, for the insertion of large DNA fragments using a single pegRNA. As described above, the subject TJ-pegRNA harbors the insertion sequence as well as two primer binding sites (PBSs), with one PBS matching a nicking sgRNA site. This example shows that TJ-PE precisely inserted 200 bp and 500 bp fragments with up to 50.5% and 11.4% efficiency, respectively, and enabled GFP (˜800 bp) insertion and expression in cells.

Prime editing is a powerful CRISPR-based genome editing approach that enables flexible genomic alterations, including all possible base substitutions, small genomic insertions, and small genomic deletions. PE usually consists of a Cas9 nickase-reverse transcriptase (RT) fusion protein and prime editing guide RNA (pegRNA). The use of two pegRNAs (e.g., TwinPE and GRAND editing) can insert relatively large DNA fragments in cells, but the efficiency of large insertions (>400 bp) remains low. Furthermore, PE shows modest efficiencies in vivo. Neither TwinPE nor GRAND editing has been applied in vivo.

For many genetic disorders, the disease-related gene can harbor diverse mutations that cause a pathogenic phenotype. Developing individual PE therapies for each pathogenic variant would be expensive and time-consuming. However, rewriting a mutation hotspot exon could provide a broadly applicable treatment strategy for genetically diverse patients. Such an approach would require PE to achieve efficient large DNA insertions.

Here, Applicant significantly improved PE by developing a template-jumping prime editor (TJ-PE) (FIGS. 1A & 1B) to enable precise insertions of large DNA fragments (up to 800 bp) at endogenous sites. As shown in this example, insertion efficiencies of up to 50.5% for 200 bp and 11.4% for 500 bp in cells have been achieved.

Specifically, a TJ-pegRNA (template jump prime editing guide RNA) and nicking sgRNA were designed as shown in FIG. 1B. The 3′ extension of TJ-pegRNA contains an insertion sequence (RTT sequence), primer binding site 1 (PBS1), and a reverse complement sequence of PBS2 (RC-PBS2, or sometimes referred to RBS2 for simplicity). After PE and TJ-pegRNA nick the top (non-targeting) DNA strand, the resulting DNA flap hybridizes to the PBS1 sequence, and the RT domain of PE synthesizes the first DNA strand. The newly synthesized DNA contains the desired insertion fragment and a PBS2 sequence at the 3′ end. PBS2 is designed to hybridize to the anchor sequence just 5′ to the second nicked site generated by PE and a nicking sgRNA to initiate the template jump and second strand synthesis.

As in Example I, the TJ-pegRNAs in this example were designed to insert 200-, 300-, or 500-bp DNA fragments into the AAVS1 locus. TJ-pegRNAs contained a trimmed evopreQ1 (tevopreQ1) motif at the 3′ end, in order to enhance pegRNA stability and improve prime editing efficiency. The TJ-pegRNA and nicking sgRNA sites were 90 bp apart, resulting in a deletion of a 90-bp genomic fragment with the desired fragment insertion.

Following transfection of HEK293T cells with TJ-pegRNA, nicking sgRNA, and PE, PCR amplification of the target region showed a band of the predicted insertion size at the AAVS1 site (FIG. 1D). Control pegRNAs were designed to produce a PBS2 complementary to a site 46 bp upstream of the nicking sgRNA site (termed PE3 control). The PE3 control showed no clear band of the predicted insertion length (FIG. 1D), suggesting that base pairing of PBS2 to the DNA flap at the nicking sgRNA site is essential for effective insertion.

Droplet digital polymerase chain reaction (ddPCR) using primers spanning the junction sequence of the insertion showed that the average insertion efficiency of TJ-PE was 50.5% for the 200-bp insertion, 35.1% for the 300-bp insertion, and 11.4% for the 500-bp insertion. The insertion efficiency of the PE3 control was 19- to 35-fold lower for the 200-, 300-, and 500-bp insertions (2.1%, 1.0%, and 0.6%, respectively; FIG. 1E) compared to TJ-PE.

To determine the accuracy of DNA fragment insertion, the PCR bands of the expected insertion sizes were gel purified. Sanger sequencing showed that these fragments were completely aligned with the expected inserted sequences (FIG. 1F).

TA cloning of individual clones with Sanger sequencing estimated accuracy rates to be 91.7%, 75.0%, and 75.0% for 200-bp, 300-bp, and 500-bp insertions, respectively (FIG. 1G and data not shown). The remaining TA clones harbor imperfect insertion or insertion with point mutations (data not shown). TA cloning shows the precise insertion in the expected insertion band.

To determine the absolute total precise insertion efficiency, PCR products were sequenced via deep sequencing. It was found that TJ-PE mediated 34.3% of accurate editing of total events for the 200-bp insertion at the AAVS1 locus (FIG. 1H).

Next, TJ-pegRNA and PE3 were compared at multiple endogenous insertion sites.

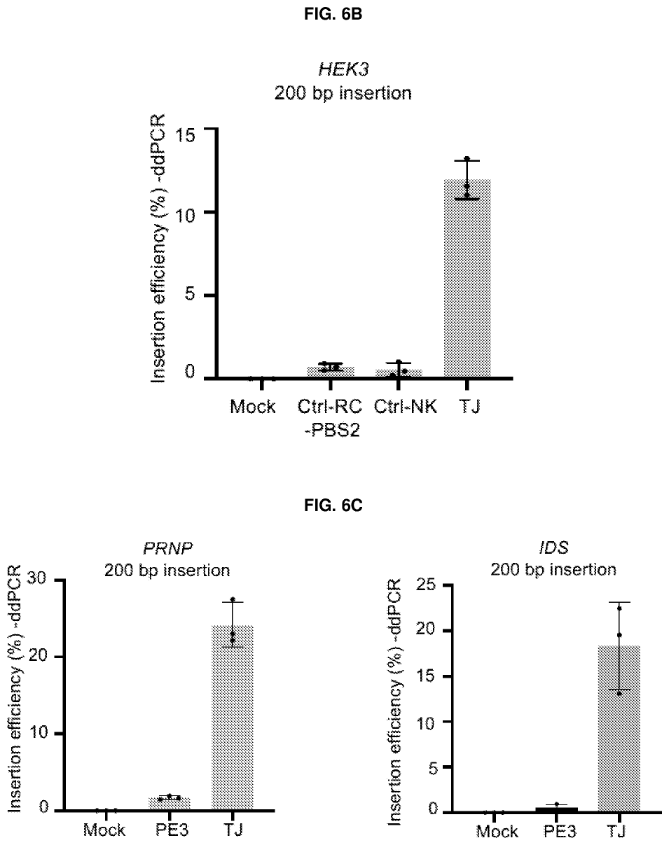

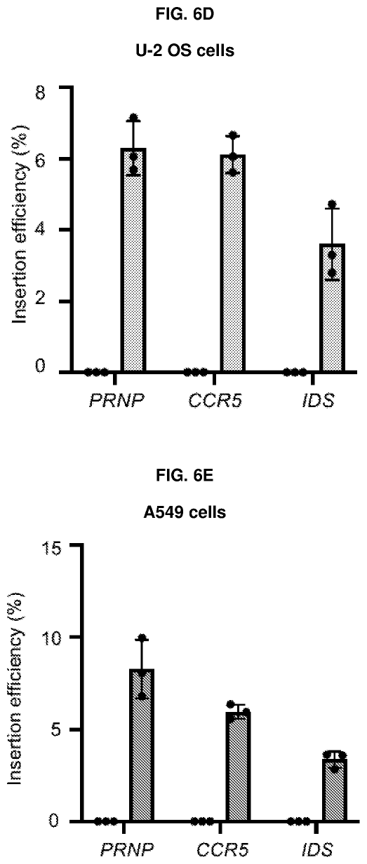

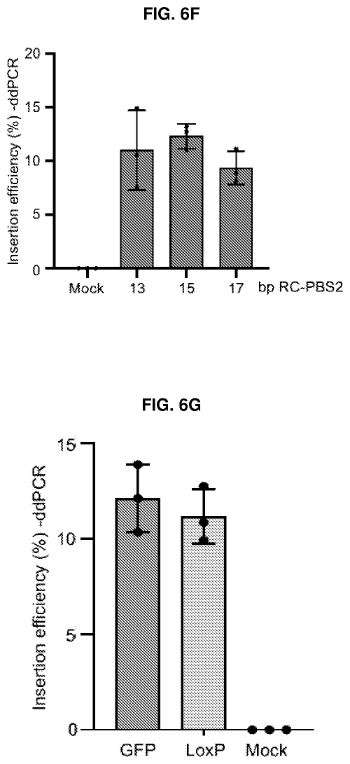

In one instance, a 200-bp DNA fragment was inserted at the endogenous HEK3 locus in HEK293 cells. The TJ-pegRNA and nicking sgRNA sites are 90 bp apart, resulting in a deletion of the 90-bp DNA fragment coupled to a 200-bp insertion. As a pegRNA control, a pegRNA was designed with an RC-PBS2 matching a sequence directly 3′ of the pegRNA nicking site (ctrl-PBS2). As a nicking sgRNA control, a nicking sgRNA (ctrl-NK) was designed to target 27 bp upstream of the site complementary to PBS2 (FIG. 6A, top panel) to generate a 63-bp deletion with a 200-bp insertion.

Using gel electrophoresis and ddPCR, the insertion efficiency of TJ-pegRNA was determined to be significantly higher than ctrl-PBS2 and ctrl-NK groups (11.9%, 0.7%, and 0.6%, respectively; FIG. 6B).

Additionally, no insertion band was detected at the HEK3 locus when the nicking sgRNA was designed to nick at the same position as ctrl-NK but on the opposite strand, indicating that the PBS2 hybridizes to the second nicked site to initiate the template jump and second-strand synthesis is essential for TJ-PE (data not shown).

Next, TJ-PE was used to insert a 200-bp fragment with concomitant 72-bp or 70-bp deletions at the endogenous PRNP or IDS loci, respectively. PegRNAs were designed to produce a PBS2 complementary to a sequence directly 3′ of the pegRNA nicking site (termed PE3 control). It was found that TJ-PE was 14-fold more efficient than PE3 at the PRNP site (24.2% versus 1.7%, respectively) and 37-fold more efficient than PE3 at the IDS site (18.4% versus 0.5%, respectively, FIG. 6C (gel image data not shown)).

The abilities of TJ-PE to support 200-bp fragment insertion in two commonly used cell lines (A549 and U-2 OS) were also tested. It was observed that TJ-PE enabled efficient genome editing (3.3%-8.3%) in both cell lines (FIGS. 6D and 6E).

To determine whether PBS2 length impacts insertion efficiency, TJ-pegRNA was designed with different RC-PBS2 lengths (13 bp, 17 bp, and 35 bp), and their abilities to insert a 200-bp fragment at the HEK3 locus were measured. All TJ-pegRNAs supported similar insertion efficiencies (11.0%, 12.3%, and 9.3%; FIG. 6F).

Furthermore, the insertions of a GFP fragment and the same sequence partially replaced by LoxP were compared. It was observed that the presence of LoxP did not impede the activity of reverse transcriptase, possibly due to the presence of RNA helicases which can potentially unwind hairpin structures in cells (FIG. 6G).

PegRNAs are sometimes prone to misfolding due to inevitable base pairing between the PBS and spacer sequence, which could potentially contribute to lower insertion efficiency. To stabilize the pegRNA and prevent misfolding, a nicking-TJ-pegRNA (NK-TJ-pegRNA) was designed to contain a PBS1 sequence that first hybridizes to the DNA flap generated by the nicking sgRNA (FIG. 10A). However, the NK-TJ-pegRNA did not increase insertion efficiency at the AAVS1 site as compared to TJ-pegRNA [62.5 versus 59.2% (for 200-bp insertion) and 41.4% versus 42.2% (for 300-bp insertion), respectively](FIGS. 10B and 10C).

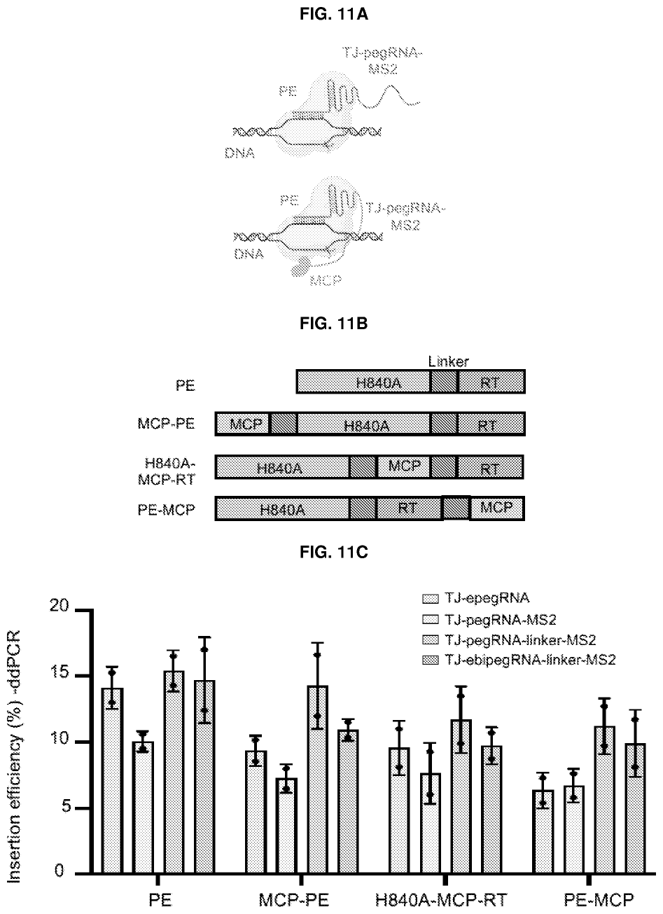

Finally, it was investigated whether tethering the PBS1 sequence of TJ-pegRNA to the PE fusion protein—via an MS2 coat protein (MCP)—improved TJ-pegRNA stability and enhanced insertion efficiency (FIGS. 11A-11C).

Specifically, the MS2 aptamer sequence was inserted at the 3′ end of TJ-pegRNA instead of the tevopreQ1 motif (FIG. 11A), and MCP was inserted into the PE fusion protein sequence (FIGS. 11A and 11B). To determine whether MCP placement affects results, different MCP fusion sites were tested in the PE protein: at the N terminus, C terminus, or between the nCas9 and RT segments of PE (FIG. 11B). It was found that, regardless of configuration, TJ-pegRNA tethered to PE-MCP protein did not increase insertion efficiency at the HEK3 locus compared to untethered TJ-pegRNA and PE (FIG. 11C).

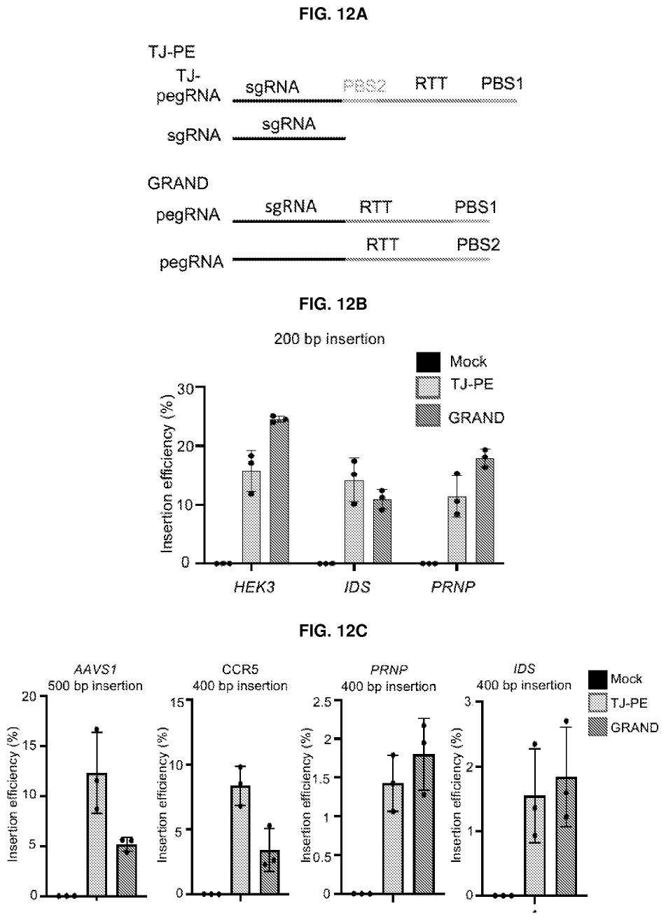

GRAND editing employs a pair of pegRNAs, which can efficiently generate the insertion of DNA fragments of less than 400 bp (FIG. 12A). The insertion efficiencies of TJ-PE and GRAND editing, in inserting a 200-bp, 400-bp or 500-bp DNA fragment at multiple endogenous sites, were compared. The results showed that TJ-PE and GRAND editing mediate similar insertion rates FIGS. 12B and 12C.

### Example III TJ-PE Mediated GFP Reporter Repair and Functional Gene Insertion

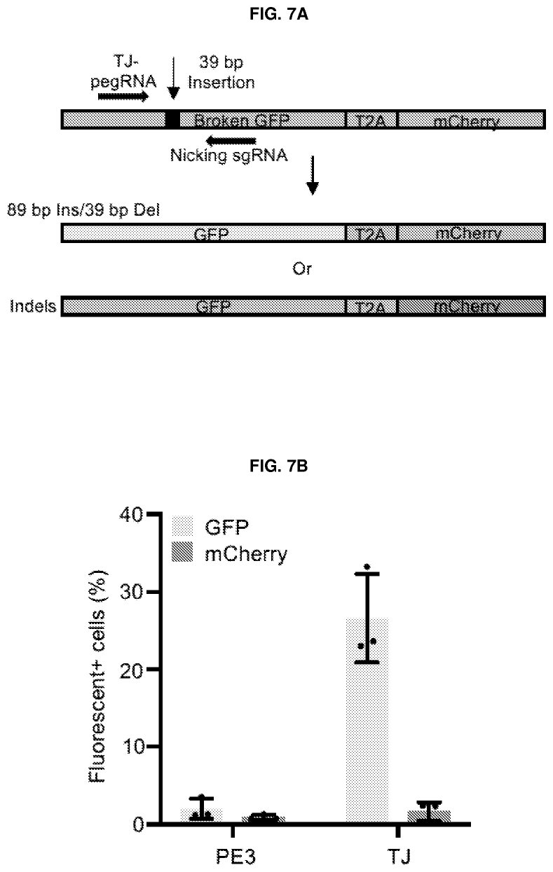

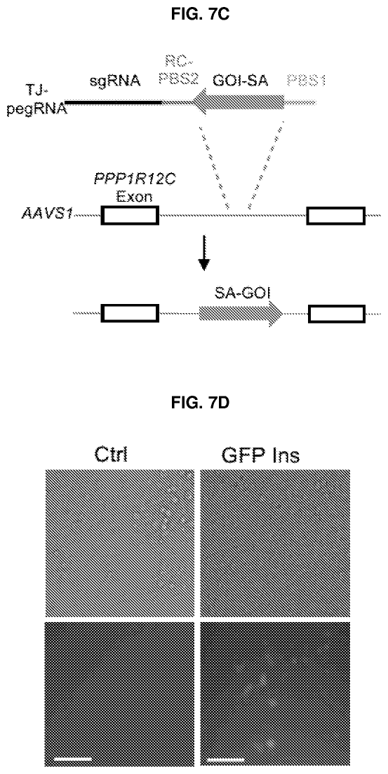

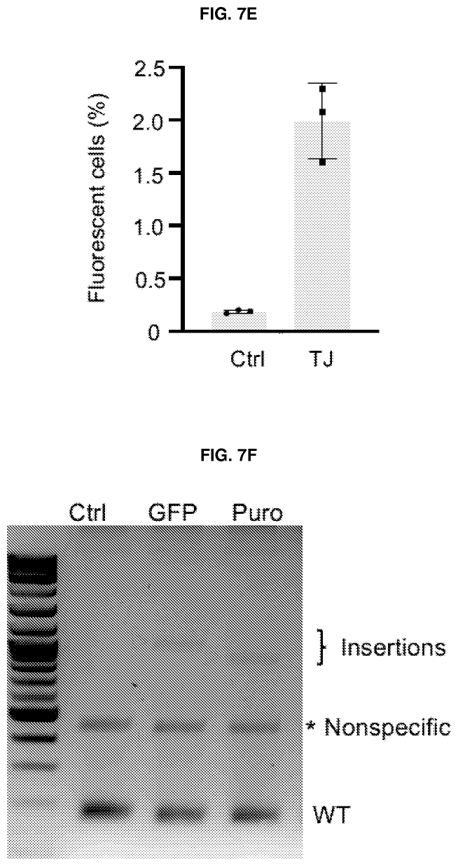

This example demonstrates that TJ-PE can mediate large in-frame insertions to restore gene expression. Specifically, the HEK293T traffic light reporter/multi-Cas variant 1 (TLR-MCV1) cell line contains a disrupted green fluorescent protein (GFP) sequence with a 39-bp sequence insertion, and an mCherry sequence, separated by a T2A sequence. The mCherry sequence is out of frame with the disrupted GFP sequence, preventing mCherry expression (FIG. 7A). Precise repair of the disrupted sequence enables GFP expression; indels that shift into the +1 reading frame will induce mCherry expression.

TLR-MCV1 cells were treated with PE, TJ-pegRNA, and nicking sgRNA designed to precisely insert an 89-bp codon-optimized fragment and concomitantly delete the 39-bp disruption sequence. A pegRNA designed to insert a 73-bp codon-optimized fragment and concomitantly delete the 39-bp disruption sequence was used as the PE3 control. TJ-PE led to a 13-fold increase in the level of precise 89-bp insertion compared to control (26.6% versus 2.0%, respectively, FIG. 7B). The indel efficiency was also higher in the TJ-PE-treated group than in the control group (1.7% versus 0.9%, respectively, FIG. 7B). These data demonstrate that TJ-PE can repair genomic coding regions through precise, large, in-frame insertions.

To demonstrate the applicability of TJ-PE with respect to different insertion sizes, TJ-pegRNA was designed to insert either splice acceptor (SA)-GFP (833 bp) or SA-Puro (709 bp) at the AAVS1 locus after deleting a 90-bp DNA fragment (FIG. 7C). Using fluorescence microscopy, it was observed that EGFP⁺ cells in the TJ-PE-treated group (FIG. 7D). Flow cytometry analysis showed that the EGFP⁺ cell efficiency was 2.0% (FIG. 7E). The control group (plasmid encoding PE protein only) showed minimal EGFP-positive cells (0.2%). After confirming insertions were the expected sizes (FIG. 7F), the insertion bands were purified and it was confirmed that these fragments were precisely inserted using Sanger sequencing (data not shown). The data demonstrate that TJ-PE can mediate functional gene insertion at AAVS1 site.

### Example IV Split Circular TJ-petRNA Enables Large Insertion for Non-Viral Delivery

This examples demonstrates that TJ-PE can be facilitated by transcription of a split circular TJ-petRNA in vitro via a permuted group I catalytic intron for non-viral delivery.

Non-viral (RNA-based) delivery of gene editors has considerable therapeutic potential for a wide range of diseases due to its many advantages, including ease of scale-up, transient expression, lack of immune response, and minimum off-target effects. However, pegRNA needs to be quite long to generate large insertions (e.g., 226-nt TJ-pegRNA is needed for a 100-bp insertion), making RNA synthesis complex. Long pegRNAs can be transcribed in vitro, but this does not allow for the addition of chemical modifications to improve pegRNA stability.

In vitro transcribed circular RNAs exhibit not only higher stability, but also lower immunogenicity, compared to unmodified linear RNA. To develop an RNA-encoded TJ-pegRNA system, TJ-pegRNA was split into an sgRNA and a prime editing template RNA (petRNA) carrying an RTT-PBS sequence (e.g., rcPBS2-RTT-PBS1) and an MS2 stem-loop aptamer (e.g., MS2-rcPBS2-RTT-PBS1, or MS2-RTT-PBS for short). The MS2-RTT-PBS was designed to form a circular RNA via a permuted group I catalytic intron in vitro (FIGS. 8A and 8E). Split circular TJ-petRNA was tethered to the MCP-RT fusion protein by the MS2 aptamer (FIG. 8B).

To test circularization efficiency, the transcribed RNA was treated with RNase R (digests linear, but not circular RNA) and RNase H. A circularization efficiency of >90% was observed (FIG. 8C). Circular RNAs were enriched using RNase R and electroporated into HEK293T cells along with sgRNA, nicking sgRNA, and mRNAs encoding nCas9 and MCP-RT. Deep sequencing showed that split circular TJ-petRNA mediates 37.6% insertion at the AAVS1 locus (FIG. 8D and data not shown).

In vitro transcribed full-length linear TJ-pegRNA was transcribed without chemical modification. FL-TJ-pegRNA showed low insertion efficiency (0.4%), likely due to the instability of unmodified RNA. Transfected TJ-pegRNA plasmid generates an accurate insertion frequency of 62.3% (FIG. 8D).

These results demonstrate that in vitro transcribed, circular MS2-containing petRNA can be coupled with TJ-PE to enable DNA fragment insertion, increasing the feasibility of using an RNA-encoded TJ-PE system to achieve large DNA insertion in vivo.

### Example V TJ-PE Mediated Recoding of the Fah Exon 8 Locus in the Tyrosinemia I Mouse Model

This example demonstrates that TJ-PE can rewrite an exon in the liver of tyrosinemia I mice to reverse the disease phenotype in vivo, demonstrating the potential of using TJ-PE to develop a broadly applicable strategy to correct large region and/or multiple pathogenic variants.

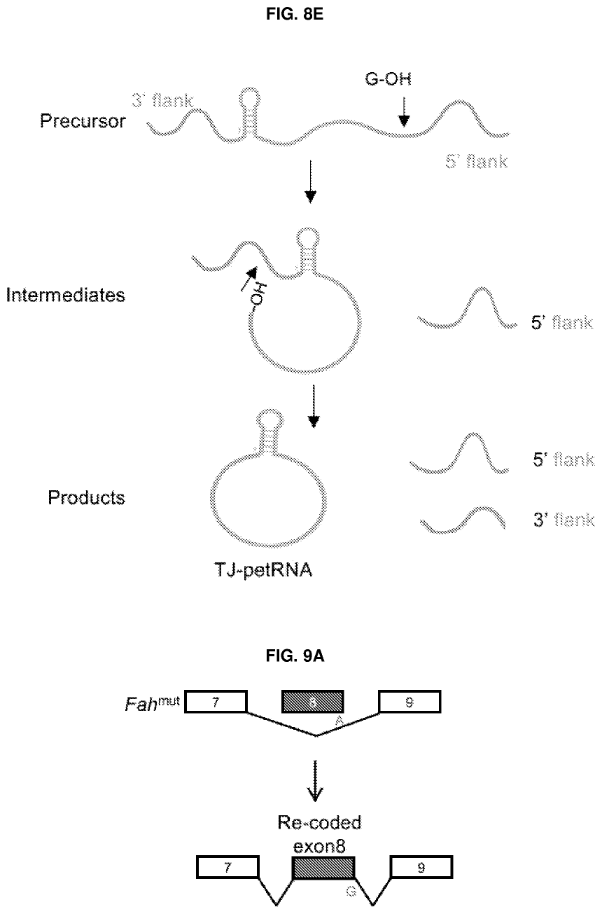

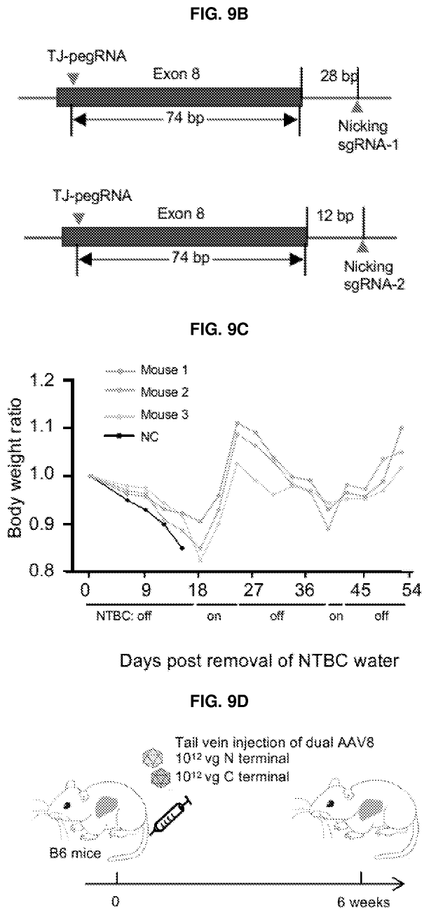

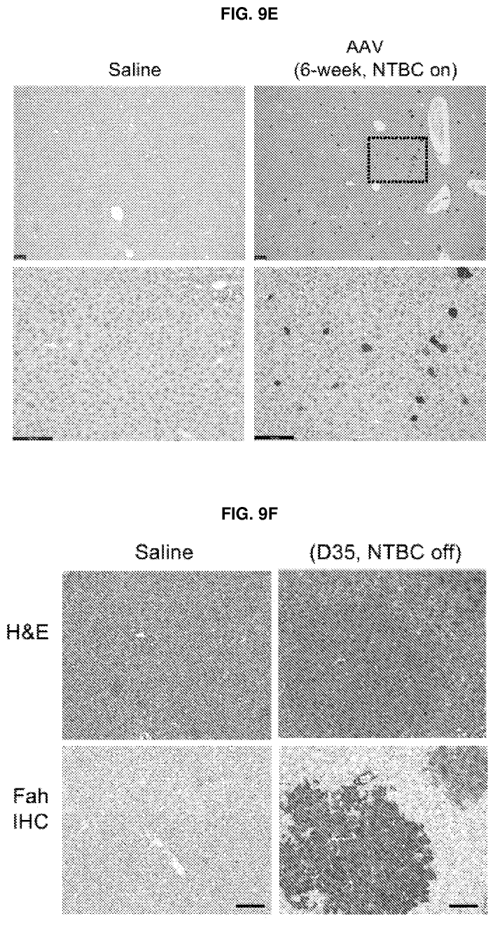

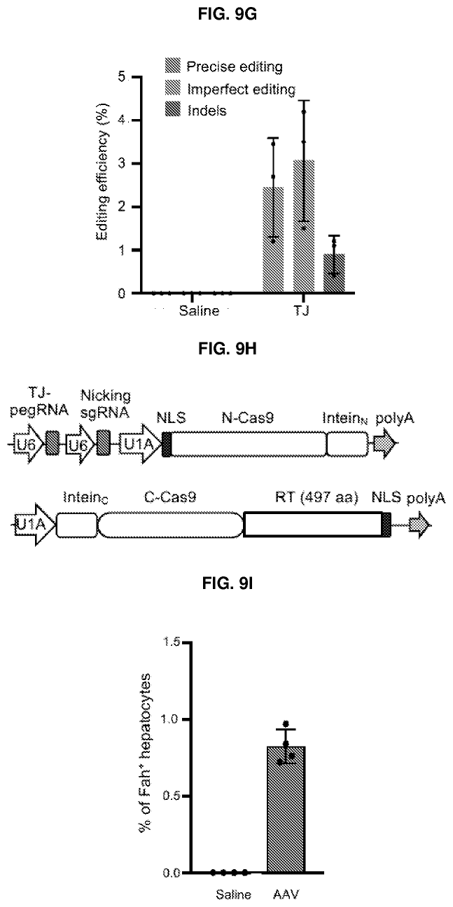

Tyrosinemia I is an autosomal recessive disorder characterized by hepatocyte toxin accumulation and liver damage. Tyrosinemia I is caused by loss-of-function mutations in the fumarylacetoacetate hydrolase (FAH) gene. Tyrosinemia I mice harbor a G·C to A·T point mutation in the last nucleotide of exon 8 in the Fah gene, resulting in exon 8 skipping and loss of functional FAH protein (FIG. 9A). Tyrosinemia I mice need to be treated with 2-(2-nitro-4-trifluoromethylbenzoyl)-1,3-cyclohexanedione (NTBC) supplemented water to maintain body weight and survive. Multiple types of mutations in exon 8 have been reported in patients.

Replacing the mutant Fah exon sequence with a synonymous DNA fragment would correct any combination of mutations in the exon. This exon rewriting strategy has the potential to correct multiple pathogenic mutations using a single template. The TJ-pegRNA and nicking sgRNA targeting the genomic region across exon 8 were engineered (FIG. 9B). TJ-pegRNA harbors the correction “G” and multiple synonymous mutations. PE2, TJ-pegRNAs, and nicking sgRNA (Nicking sgRNA-1) plasmids were delivered to the livers of mice via hydrodynamic injection. FAH-expressing hepatocytes were detected on TJ-PE-treated liver sections with a 0.1% correction rate (data not shown) two weeks after hydrodynamic injection. Since hepatocytes with corrected FAH protein gain a growth advantage, the NTBC supplement was removed, and it was observed that Fah-mutant mice treated with saline control rapidly lost 15% of body weight, while TJ-PE showed body weight rescued 45 days after NTBC withdrawal (FIG. 9C). Widespread Fah-positive cell clusters were observed in TJ-PE-treated mouse livers by immunohistochemistry (FIG. 9F).

The efficiency of precise replacement was confirmed via deep sequencing two months after NTBC withdrawal (average 3.1%, FIG. 9G). Also observed was sequencing reads with partial synonymous mutations and/or the correction “G” incorporated (data not shown), which may be due to that the RTT with synonymous mutations is highly homologous to the genomic sequence.

To reduce the imperfect editing and improve precise editing, the RTT was optimized further to avoid microhomology with the genomic sequence and used a new nicking sgRNA (Nicking sgRNA-2) which is closer to the rewritten exon to include less intron sequence (FIG. 9B). TJ-PE was delivered using the dual-AAV8 split-intein system to Fah-mutant mice that were kept on NTBC-supplemented water for 6-week to prevent the expansion of Fah-corrected cells (FIG. 9D & 9H). Up to 1.0% of hepatocytes stained positive for the FAH protein by immunohistochemistry in AAV-treated animals (FIGS. 9E and 9I).

Overall, the data demonstrate the potential of using TJ-PE in vivo to insert large DNA fragments without double-stranded DNA breaks, and to facilitate mutation hotspot exon rewriting in vivo.

The following experimental details used in Examples II-V are provided below for illustrative purpose only, and is not in any way limiting to the general principle of the invention described herein. However, specific embodiments described in these experiments are all part of the general disclosure of the invention, and can be combined with any one or more embodiments of the invention.

### Plasmid Construction

Plasmids expressing sgRNA were constructed by ligation of annealed oligonucleotides into a custom vector (BfuAI digested). To generate pegRNA plasmids, gBlocks gene fragments (spacer, scaffold, and 3′ extension sequences) were synthesized by Integrated DNA Technologies, and subsequently cloned into a BfuAI/EcoRI-digested vector by Gibson assembly. The PE-Sto7d plasmid was constructed through Gibson assembly with PE2 digested by AgeI and EcoRI. Codon-optimized Sto7d, NC domain was synthesized by Integrated DNA Technologies. Sequences of sgRNA and pegRNA are listed in Table 1. Plasmids used for in vitro experiments were purified using Miniprep kits (Qiagen). Plasmids were purified using a Maxiprep kit (Qiagen) including the endotoxin removal step for in vivo experiments.

### Cell Culture, Transfection and Genomic DNA Isolation

HEK293T cells acquired from ATCC were maintained in Dulbecco's Modified Eagle's Medium (DMEM) supplemented with 10% (v/v) fetal bovine serum (Gibco) and 1% (v/v) Penicillin/Streptomycin (Gibco). Cells were cultured at 37° C. with 5% CO₂.

HEK293T cells were seeded on 12-well plates overnight at 100,000 cells per well. One microgram PE2, 500 ng pegRNA, and 500 ng nicking sgRNA were transfected using Lipofectamine 3000 (Invitrogen). Cells were collected 4 days after transfection, lysed with 100 μL Quick extraction buffer (Epicenter), and incubated on a thermocycler at 65° C. for 15 min and 98° C. for 5 min. Sequences of primers used for genomic DNA amplification are listed in Table 2.

Droplet Digital PCR (ddPCR)

ddPCR was used to quantify the amplicon containing the insertion fragment (HEK3, IDS and PRNP loci) or insertion-genome junction (AAVS1) in comparison to a reference amplicon. Briefly, gDNA was added to a reaction containing ddPCR Supermix (no dUTP, Bio-Rad), the primers (900 nM) and the probes (250 nM). Droplets were generated using a QX200 Manual Droplet Generator (Bio-Rad). PCR reactions were carried out as follows: 95° C. for 10 min, 36 cycles of 94° C. for 30 s and 58° C. for 1 min, 98° C. for 10 min, and 4° C. holds. Droplets were read using a QX200 Droplet Reader (Bio-Rad) and analyzed using QuantaSoft (Bio-Rad). Sequences of probes are listed in Table 3.

### Flow Cytometry Analysis

Flow cytometry analysis was performed on day 4 after transfection. Reporter cells were collected after PBS washing and trypsin digestion and resuspended in PBS with 2% FBS for flow cytometry analysis (MACSQuant VYB). Data were analyzed by FlowJo 10.0 software.

### In Vitro Transcription

The transcription of split circular TJ-petRNA was performed as known in the art. The template was synthesized by Integrated DNA Technologies and amplified via PCR. Split circular TJ-petRNA was generated at 37° C. for 4 h using a HiScribe T7 High-Yield RNA Kit (New England Biolabs) according to the manufacturer's protocol. After DNase I digest, 0.8 μL 100 mM GTP was added to 1 reaction, 55° C. for 15 min. The RNA was then purified using a Monarch RNA Cleanup kit (New England Biolabs).

### Nucleofection

The Neon electroporation system was used for electroporation. Briefly, 1 g of each mRNA, 100 pmol of sgRNA, 100 pmol of nicking sgRNA, and 30 pmol split circular TJ-petRNA were electroporated into 5×104 HEK293T cells. One microgram of each mRNA, 100 pmol of pegRNA, and 100 pmol of nicking sgRNA was electroporated as control group. HEK293T cells were electroporated using the following electroporation parameters: 1,150 V, 20 ms, two pulses.

### Deep Sequencing and Data Analysis

Sequencing library preparation was performed as previously described. Briefly, for the first round of PCR, the primers containing Illumina forward and reverse adapters (listed in Table 4) were used for amplifying the genomic sites of interest from 100 ng genomic DNA using Phusion Hot Start II PCR Master Mix. PCR 1 reactions were carried out as follows: 98° C. for 10 s, then 20 cycles of 98° C. for 1 s, 58° C. for 5 s, and 72° C. for 6 s, followed by a final 72° C. extension for 2 min. A secondary PCR reaction were performed to add a unique Illumina barcode to each sample from 1 μL unpurified PCR 1 product. PCR 2 reactions were carried out as follows: 98° C. for 10 s, then 20 cycles of 98° C. for 1 s, 60° C. for 5 s, and 72° C. for 8 s, followed by a final 72° C. extension for 2 min. PCR 2 products were purified by gel purification using the QIAquick Gel Extraction Kit (Qiagen). DNA concentration was measured by Qubit dsDNA HS Assay Kit (Thermo Fisher Scientific). The library was sequenced on an Illumina MiniSeq instrument following the manufacturer's protocols. Sequencing reads were demultiplexed using bcl2fastq (Illumina). To quantify the frequency of precise editing and indels, CRISPResso230 was run in HDR mode with “plot_window_size”=65, “default_min_aln_score”=60, ‘min_average_read_quality’=30. The indel efficiency was calculated as 100%—precise insertion—WT, and then normalize to a blank group.

### Animal Studies

All animal experiments were approved by the Institutional Animal Care and Use Committee (IACUC) at University of Massachusetts Chan Medical School (PROT0202000051). All plasmids used for hydrodynamic tail-vein injection were prepared using EndoFree Plasmid Maxi kit (Qiagen). Fah mutant mice were kept on 10 mg/L NTBC water. Thirty micrograms of PE2, 15 μg TJ-pegRNA, and 15 μg nicking sgRNA were injected into Fah mutant mice via the tail vein in 5-7 s. Saline were injected in the control group. NTBC-supplemented water was replaced with normal water 7-14 days after injection, and mouse weight was measured daily.

### AAV Production

Low-passage HEK293T cells were transfected with AAV genome, pHelper, and Rep/Cap plasmids using PEI. After three days, the cells were dislodged and transferred to 50 mL Falcon tubes. For AAV purification, 1/10 Volume of pure chloroform was added and shaken vigorously at 37° C. for 1 h. NaCl was added to a final concentration of 1M, followed by centrifugation at 20,000 g at 4° C. for 15 min. The supernatant was gently collected and PEG8000 (Sigma) was added for virus precipitation. The pellet was resuspended in DPBS containing MgCl₂ and Benzonase (Sigma), and incubated at 37° C. for 45 min. Chloroform was added to remove protein and the aqueous layer was ultrafiltered through 100 kDa MWCO columns (Millipore). The virus titer was quantified via qPCR31.

### Data Availability

The raw sequencing data have been deposited to the NCBI BioProject database. All raw data are available from the corresponding author upon request.

### REFERENCES

1. Anzalone, A. V. et al. Search-and-replace genome editing without double-strand breaks or donor DNA. Nature 576, 149-157 (2019).
2. Anzalone, A. V., Koblan, L. W. & Liu, D. R. Genome editing with CRISPR-Cas nucleases, base editors, transposases and prime editors. Nat Biotechnol 38, 824-844 (2020).
3. Chen, P. J. et al. Enhanced prime editing systems by manipulating cellular determinants of editing outcomes. Cell 184, 5635-5652 e5629 (2021).
4. Zong, Y. et al. An engineered prime editor with enhanced editing efficiency in plants. Nat Biotechnol (2022).
5. Anzalone, A. V. et al. Programmable deletion, replacement, integration and inversion of large DNA sequences with twin prime editing. Nat Biotechnol 40, 731-740 (2022).
6. Wang, J. et al. Efficient targeted insertion of large DNA fragments without DNA donors. Nat Methods 19, 331-340 (2022).
7. Zheng, C. et al. A flexible split prime editor using truncated reverse transcriptase improves dual-AAV delivery in mouse liver. Mol Ther 30, 1343-1351 (2022).
8. Raguram, A., Banskota, S. & Liu, D. R. Therapeutic in vivo delivery of gene editing agents. Cell 185, 2806-2827 (2022).
9. Liu, B. et al. A split prime editor with untethered reverse transcriptase and circular RNA template. Nat Biotechnol (2022).
10. Bock, D. et al. In vivo prime editing of a metabolic liver disease in mice. Sci Transl Med 14, eabl9238 (2022).
11. McClellan, J. & King, M. C. Genetic heterogeneity in human disease. Cell 141, 210-217 (2010).
12. Landrum, M. J. et al. ClinVar: public archive of relationships among sequence variation and human phenotype. Nucleic Acids Res 42, D980-985 (2014).
13. Shastry, B. S. SNPs in disease gene mapping, medicinal drug development and evolution. J Hum Genet 52, 871-880 (2007).
14. Han, J. S. Non-long terminal repeat (non-LTR) retrotransposons: mechanisms, recent developments, and unanswered questions. Mob DNA 1, 15 (2010).
15. Gorbunova, V. et al. The role of retrotransposable elements in ageing and age-associated diseases. Nature 596, 43-53 (2021).
16. Nelson, J. W. et al. Engineered pegRNAs improve prime editing efficiency. Nat Biotechnol 40, 402-410 (2022).
17. Liu, Y. et al. Enhancing prime editing by Csy4-mediated processing of pegRNA. Cell Res 31, 1134-1136 (2021).
18. Iyer, S. et al. Efficient Homology-Directed Repair with Circular Single-Stranded DNA Donors. CRISPR J (2022).
19. Yin, H. et al. Structure-guided chemical modification of guide RNA enables potent non-viral in vivo genome editing. Nat Biotechnol 35, 1179-1187 (2017).
20. Wesselhoeft, R. A. et al. RNA Circularization Diminishes Immunogenicity and Can Extend Translation Duration In Vivo. Mol Cell 74, 508-520 e504 (2019).
21. Kay, M. A. State-of-the-art gene-based therapies: the road ahead. Nat Rev Genet 12, 316-328 (2011).
22. Wesselhoeft, R. A., Kowalski, P. S. & Anderson, D. G. Engineering circular RNA for potent and stable translation in eukaryotic cells. Nat Commun 9, 2629 (2018).
23. Petkovic, S. & Muller, S. RNA circularization strategies in vivo and in vitro. Nucleic Acids Res 43, 2454-2465 (2015).
24. Holme, E. & Lindstedt, S. Diagnosis and management of tyrosinemia type I. Curr Opin Pediatr 7, 726-732 (1995).
25. Paulk, N. K. et al. Adeno-associated virus gene repair corrects a mouse model of hereditary tyrosinemia in vivo. Hepatology 51, 1200-1208 (2010).
26. Angileri, F. et al. Geographical and Ethnic Distribution of Mutations of the Fumarylacetoacetate Hydrolase Gene in Hereditary Tyrosinemia Type 1. JIMD reports 19, 43-58 (2015).
27. Song, M. et al. Generation of a more efficient prime editor 2 by addition of the Rad51 DNA-binding domain. Nat Commun 12, 5617 (2021).
28. Ioannidi, E. I. et al. Drag-and-drop genome insertion without DNA cleavage with CRISPR-directed integrases
   Preprint at https://biorxiv.org/content/10.1101/2021.11.01.466786v1 (2021).
29. Liu, P. et al. Improved prime editors enable pathogenic allele correction and cancer modelling in adult mice. Nat Commun 12, 2121 (2021).
30. Clement, K. et al. CRISPResso2 provides accurate and rapid genome editing sequence analysis. Nat Biotechnol 37, 224-226 (2019).
31. Su, Q., Sena-Esteves, M. & Gao, G. Titration of Recombinant Adeno-Associated Virus (rAAV) Genome Copy Number Using Real-Time Quantitative Polymerase Chain Reaction (qPCR). Cold Spring Harb Protoc 2020, 095646 (2020).

All references cited herein are incorporated by reference in their entirety, preferably incorporated at the place of citation.

### Sequences

TABLE 1
Sequences of pegRNAs and sgRNAs used in Examples II-V.
sgRNA scaffold sequence
GTTTTAGAGCTAGAAATAGCAAGTTAAAATAAGGCTAGTCCGTTATCAACTTGAAAAAGTGG
GACCGAGTCGGTCC (SEQ ID NO: 4)

| | spacer | SEQ | | SEQ | |
|---|---|---|---|---|---|
| | sequence | ID | | ID | |
| pegRNA | (5′-3′) | NO | 3′ extension | NO | Figure |
| AAVS1 200 bp | GATGGAGCC | 5 | GCAGCTCAGGTTCTGGGATAACTTCGTAT | 6 | FIG. 1B |
| insertion TJ- | AGAGAGGAT | | AATGTATGCTATACGAAGTTATAACAATC | | |
| pegRNA | CC | | CCCCAACTGAGAGAACTCAAAGGTTACCC | | |
| | | | CAGTTGGGGCACATTACCCTGTTATCCCT | | |
| | | | ACCTCGCCGGACACGCTGAACTTGTGGCC | | |
| | | | GTTTACGTCGCCGTCCAGCTCGACCAGGA | | |
| | | | TGGGCACCACCCCGGTGAACAGCTCCTCG | | |
| | | | CCCTTGCTCACCATTCCTCTCTGGCTCTC | | |
| | | | TCTCTCTTGACGCGGTTCTATCTAGTTAC | | |
| | | | GCGTTAAACCAACTAGAAATTTTTTT | | |
| AAVS1 200 bp | GATGGAGCC | 7 | CCGGGCAGGTCACGCATATAACTTCGTAT | 8 | FIG. 1B |
| insertion | AGAGAGGAT | | AATGTATGCTATACGAAGTTATAACAATC | | |
| pegRNA | CC | | CCCCAACTGAGAGAACTCAAAGGTTACCC | | |
| | | | CAGTTGGGGCACATTACCCTGTTATCCCT | | |
| | | | ACCTCGCCGGACACGCTGAACTTGTGGCC | | |
| | | | GTTTACGTCGCCGTCCAGCTCGACCAGGA | | |
| | | | TGGGCACCACCCCGGTGAACAGCTCCTCG | | |
| | | | CCCTTGCTCACCATTCCTCTCTGGCTCTC | | |
| | | | TCTCTCTTGACGCGGTTCTATCTAGTTAC | | |
| | | | GCGTTAAACCAACTAGAAATTTTTTT | | |
| AAVS1 300 bp | GATGGAGCC | 9 | GCAGCTCAGGTTCTGGGATAACTTCGTAT | 10 | FIG. 1B |
| insertion TJ- | AGAGAGGAT | | AATGTATGCTATACGAAGTTATAACAATC | | |
| pegRNA | CC | | CCCCAACTGAGAGAACTCAAAGGTTACCC | | |
| | | | CAGTTGGGGCACATTACCCTGTTATCCCT | | |
| | | | ATAGGTCAGGGTGGTCACGAGGGTGGGCC | | |
| | | | AGGGCACGGGCAGCTTGCCGGTGGTGCAG | | |
| | | | ATGAACTTCAGGGTCAGCTTGCCGTAGGT | | |
| | | | GGCATCGCCCTCGCCCTCGCCGGACACGC | | |
| | | | TGAACTTGTGGCCGTTTACGTCGCCGTCC | | |
| | | | AGCTCGACCAGGATGGGCACCACCCCGGT | | |
| | | | GAACAGCTCCTCGCCCTTGCTCACCATTC | | |
| | | | CTCTCTGGCTCTCTCTCTCTTGACGCGGT | | |
| | | | TCTATCTAGTTACGCGTTAAACCAACTAG | | |
| | | | AAATTTTTTT | | |
| AAVS1 300 bp | GATGGAGCC | 11 | CCGGGCAGGTCACGCATATAACTTCGTAT | 12 | FIG. 1B |
| insertion | AGAGAGGAT | | AATGTATGCTATACGAAGTTATAACAATC | | |
| pegRNA | CC | | CCCCAACTGAGAGAACTCAAAGGTTACCC | | |
| | | | CAGTTGGGGCACATTACCCTGTTATCCCT | | |
| | | | ATAGGTCAGGGTGGTCACGAGGGTGGGCC | | |
| | | | AGGGCACGGGCAGCTTGCCGGTGGTGCAG | | |
| | | | ATGAACTTCAGGGTCAGCTTGCCGTAGGT | | |
| | | | GGCATCGCCCTCGCCCTCGCCGGACACGC | | |
| | | | TGAACTTGTGGCCGTTTACGTCGCCGTCC | | |
| | | | AGCTCGACCAGGATGGGCACCACCCCGGT | | |
| | | | GAACAGCTCCTCGCCCTTGCTCACCATTC | | |
| | | | CTCTCTGGCTCTCTCTCTCTTGACGCGGT | | |
| | | | TCTATCTAGTTACGCGTTAAACCAACTAG | | |
| | | | AAATTTTTTT | | |
| AAVS1 500 bp | GATGGAGCC | 13 | GCAGCTCAGGTTCTGGGATAACTTCGTAT | 14 | FIG. 1B |
| insertion TJ- | AGAGAGGAT | | AATGTATGCTATACGAAGTTATAACAATC | | |
| pegRNA | CC | | CCCCAACTGAGAGAACTCAAAGGTTACCC | | |
| | | | CAGTTGGGGCACATTACCCTGTTATCCCT | | |
| | | | ACCTCCTTGAAGTCGATGCCCTTCAGCTC | | |
| | | | GATGCGGTTCACCAGGGTGTCGCCCTCGA | | |
| | | | ACTTCACCTCGGCGCGGGTCTTGTAGTTG | | |
| | | | CCGTCGTCCTTGAAGAAGATGGTGCGCTC | | |
| | | | CTGGACGTAGCCTTCGGGCATGGCGGACT | | |
| | | | TGAAGAAGTCGTGCTGCTTCATGTGGTCG | | |
| | | | GGGTAGCGGCTGAAGCACTGCACGCCGTA | | |
| | | | GGTCAGGGTGGTCACGAGGGTGGGCCAGG | | |
| | | | GCACGGGCAGCTTGCCGGTGGTGCAGATG | | |
| | | | AACTTCAGGGTCAGCTTGCCGTAGGTGGC | | |
| | | | ATCGCCCTCGCCCTCGCCGGACACGCTGA | | |
| | | | ACTTGTGGCCGTTTACGTCGCCGTCCAGC | | |
| | | | TCGACCAGGATGGGCACCACCCCGGTGAA | | |
| | | | CAGCTCCTCGCCCTTGCTCACCATTCCTC | | |
| | | | TCTGGCTCTCTCTCTCTTGACGCGGTTCT | | |
| | | | ATCTAGTTACGCGTTAAACCAACTAGAAA | | |
| | | | TTTTTTT | | |
| AAVS1 500 bp | GATGGAGCC | 15 | CCGGGCAGGTCACGCATATAACTTCGTAT | 16 | FIG. 1B |
| insertion | AGAGAGGAT | | AATGTATGCTATACGAAGTTATAACAATC | | |
| pegRNA | CC | | CCCCAACTGAGAGAACTCAAAGGTTACCC | | |
| | | | CAGTTGGGGCACATTACCCTGTTATCCCT | | |
| | | | ACCTCCTTGAAGTCGATGCCCTTCAGCTC | | |
| | | | GATGCGGTTCACCAGGGTGTCGCCCTCGA | | |
| | | | ACTTCACCTCGGCGCGGGTCTTGTAGTTG | | |
| | | | CCGTCGTCCTTGAAGAAGATGGTGCGCTC | | |
| | | | CTGGACGTAGCCTTCGGGCATGGCGGACT | | |
| | | | TGAAGAAGTCGTGCTGCTTCATGTGGTCG | | |
| | | | GGGTAGCGGCTGAAGCACTGCACGCCGTA | | |
| | | | GGTCAGGGTGGTCACGAGGGTGGGCCAGG | | |
| | | | GCACGGGCAGCTTGCCGGTGGTGCAGATG | | |
| | | | AACTTCAGGGTCAGCTTGCCGTAGGTGGC | | |
| | | | ATCGCCCTCGCCCTCGCCGGACACGCTGA | | |
| | | | ACTTGTGGCCGTTTACGTCGCCGTCCAGC | | |
| | | | TCGACCAGGATGGGCACCACCCCGGTGAA | | |
| | | | CAGCTCCTCGCCCTTGCTCACCATTCCTC | | |
| | | | TCTGGCTCTCTCTCTCTTGACGCGGTTCT | | |
| | | | ATCTAGTTACGCGTTAAACCAACTAGAAA | | |
| | | | TTTTTTT | | |
| HEK3 200 bp | GGCCCAGAC | 17 | GTCAACCAGTATCCCGGATGGTGAGCAAG | 18 | FIG. 1B |
| insertion TJ- | TGAGCACGT | | GGCGAGGAGCTGTTCACCGGGGTGGTGCC | | |
| pegRNA | GA | | CATCCTGGTCGAGCTGGACGGCGACGTAA | | |
| | | | ACGGCCACAAGTTCAGCGTGTCCGGCGAG | | |
| | | | GGCGAGGGCGATGCCACCTACGGCAAGCT | | |
| | | | GACCCTGAAGTTCATCTGCACCACCGGCA | | |
| | | | AGCTGCCCGTGCCCTGGCCCACCCTCGTG | | |
| | | | ACCACCCTGACCTACGTGCTCAGTCTGTA | | |
| | | | GACACACGCGGTTCTATCTAGTTACGCGT | | |
| | | | TAAACCAACTAGAATTTTTTT | | |
| HEK3 200 bp | GGCCCAGAC | 19 | CCTTTCCTCTGCCATCAATGGTGAGCAAG | 20 | FIG. 6A |
| insertion | TGAGCACGT | | GGCGAGGAGCTGTTCACCGGGGTGGTGCC | | & FIG. |
| control PBS2 | GA | | CATCCTGGTCGAGCTGGACGGCGACGTAA | | 6B |
| pegRNA | | | ACGGCCACAAGTTCAGCGTGTCCGGCGAG | | |
| | | | GGCGAGGGCGATGCCACCTACGGCAAGCT | | |
| | | | GACCCTGAAGTTCATCTGCACCACCGGCA | | |
| | | | AGCTGCCCGTGCCCTGGCCCACCCTCGTG | | |
| | | | ACCACCCTGACCTACGTGCTCAGTCTGTA | | |
| | | | GACACACGCGGTTCTATCTAGTTACGCGT | | |
| | | | TAAACCAACTAGAATTTTTTT | | |
| PRNP 200 bp | GCAGTGGTG | 21 | GCATGTTTTCACGATAGATGGTGAGCAAG | 22 | FIG. 6C |
| insertion TJ- | GGGGGCCTT | | GGCGAGGAGCTGTTCACCGGGGTGGTGCC | | |
| pegRNA | GG | | CATCCTGGTCGAGCTGGACGGCGACGTAA | | |
| | | | ACGGCCACAAGTTCAGCGTGTCCGGCGAG | | |
| | | | GGCGAGGGCGATGCCACCTACGGCAAGCT | | |
| | | | GACCCTGAAGTTCATCTGCACCACCGGCA | | |
| | | | AGCTGCCCGTGCCCTGGCCCACCCTCGTG | | |
| | | | ACCACCCTGACCTAAGGCCCCCCACCATC | | |
| | | | TCTCTCTTGACGCGGTTCTATCTAGTTAC | | |
| | | | GCGTTAAACCAACTAGAAATTTTTTT | | |
| PRNP 200 bp | GCAGTGGTG | 23 | CCAGCATGTAGCCGCCAATGGTGAGCAAG | 24 | FIG. 6C |
| insertion | GGGGGCCTT | | GGCGAGGAGCTGTTCACCGGGGTGGTGCC | | |
| pegRNA | GG | | CATCCTGGTCGAGCTGGACGGCGACGTAA | | |
| | | | ACGGCCACAAGTTCAGCGTGTCCGGCGAG | | |
| | | | GGCGAGGGCGATGCCACCTACGGCAAGCT | | |
| | | | GACCCTGAAGTTCATCTGCACCACCGGCA | | |
| | | | AGCTGCCCGTGCCCTGGCCCACCCTCGTG | | |
| | | | ACCACCCTGACCTAAGGCCCCCCACCATC | | |
| | | | TCTCTCTTGACGCGGTTCTATCTAGTTAC | | |
| | | | GCGTTAAACCAACTAGAAATTTTTTT | | |
| IDS 200 bp | GCATTTTCG | 25 | ACTGAGGGATGTCTGAAATGGTGAGCAAG | 26 | FIG. 6C |
| insertion TJ- | ATTCCGTGA | | GGCGAGGAGCTGTTCACCGGGGTGGTGCC | | |
| pegRNA | CT | | CATCCTGGTCGAGCTGGACGGCGACGTAA | | |
| | | | ACGGCCACAAGTTCAGCGTGTCCGGCGAG | | |
| | | | GGCGAGGGCGATGCCACCTACGGCAAGCT | | |
| | | | GACCCTGAAGTTCATCTGCACCACCGGCA | | |
| | | | AGCTGCCCGTGCCCTGGCCCACCCTCGTG | | |
| | | | ACCACCCTGACCTACACGGAATCGAAATC | | |
| | | | TCTCTCTTGACGCGGTTCTATCTAGTTAC | | |
| | | | GCGTTAAACCAACTAGAAATTTTTTT | | |
| IDS 200 bp | GCATTTTCG | 27 | CGGATCCTCTTCCAAGTATGGTGAGCAAG | 28 | FIG. 6C |
| insertion | ATTCCGTGA | | GGCGAGGAGCTGTTCACCGGGGTGGTGCC | | |
| pegRNA | CT | | CATCCTGGTCGAGCTGGACGGCGACGTAA | | |
| | | | ACGGCCACAAGTTCAGCGTGTCCGGCGAG | | |
| | | | GGCGAGGGCGATGCCACCTACGGCAAGCT | | |
| | | | GACCCTGAAGTTCATCTGCACCACCGGCA | | |
| | | | AGCTGCCCGTGCCCTGGCCCACCCTCGTG | | |
| | | | ACCACCCTGACCTACACGGAATCGAAATC | | |
| | | | TCTCTCTTGACGCGGTTCTATCTAGTTAC | | |
| | | | GCGTTAAACCAACTAGAAATTTTTTT | | |
| GFP 89 bp | AGTTCAGCG | 29 | GTCAGGGTGGTCACGAGCGTAGGCCAGGG | 30 | FIG. 7A |
| replacement | TGTCCGGCT | | AACAGGCAGCTTACCCGTTGTGCAAATGA | | & FIG. |
| TJ-pegRNA | T | | ATTTGAGTGTGAGTTTCCCATAGGTGGCA | | 7B |
| | | | TCGCCCTCGCCCTCGCCGGACACGCTGAT | | |
| | | | AAATATCTTGACGCGGTTCTATCTAGTTA | | |
| | | | CGCGTTAAACCAACTAGAAATTTTTTT | | |
| SA-Puro | GATGGAGCC | 31 | GCAGCTCAGGTTCTGGGTCAGGCACCGGG | 32 | FIG. 7C |
| insertion | AGAGAGGAT | | CTTGCGGGTCATGCACCAGGTGCGCGGTC | | & FIG. |
| TJ-pegRNA | CC | | CTTCGGGCACCTCGACGTCGGCGGTGACG | | 7F |
| | | | GTGAAGCCGAGCCGCTCGTAGAAGGGGAG | | |
| | | | GTTGTGGGGCGCGGAGGTCTCCAGGAAGG | | |
| | | | CGGGCACCCCGGCGCGCTCGGCCGCCTCC | | |
| | | | ACTCCGGGGAGCACGACGGCGCTGCCCAG | | |
| | | | ACCCTTGCCCTGGTGGTCGGGCGAGACGC | | |
| | | | CGACGGTGGCCAGGAACCACGCGGGCTCC | | |
| | | | TTGGGCCGGTGCGGCGCCAGGAGGCCTTC | | |
| | | | CATCTGTTGCTGCGCGGCCAGCCGGGAAC | | |
| | | | CGCTCAACTCGGCCATGCGCGGGCCGATC | | |
| | | | TCGGCGAACACCGCCCCCGCTTCGACGCT | | |
| | | | CTCCGGCGTGGTCCAGACCGCCACCGCGG | | |
| | | | CGCAGTCGTCCGCGACCCACACCTTGTCG | | |
| | | | ATGTCGAGCCCGACGCGCGTGAGGAAGAG | | |
| | | | TTCTTGCAGCTCGGTGACCCGCTCGATGT | | |
| | | | GGCGGTCCGGATCGACGGTGTGGCGCGTG | | |
| | | | GCGGGGTAGTCGGCGAACGCGGCGGCGAG | | |
| | | | GGTGCGTACGGCCCTGGGGACGTCGTCGC | | |
| | | | GGGTGGCGAGGCGCACCGTGGGCTTGTAC | | |
| | | | TCGGTCATTGGGCCAGGATTCTCCTCGAC | | |
| | | | GTCACCGCATGTTAGCAGACTTCCTCTGC | | |
| | | | CCTCTCCGCTGCCAGATCTCTCGAGGCCC | | |
| | | | TGTGGGAGGAAGAGAAGAGGTCAGAAGCT | | |
| | | | TTCCTCTCTGGCTCTCTCTCTCTTGACGC | | |
| | | | GGTTCTATCTAGTTACGCGTTAAACCAAC | | |
| | | | TAGAAATTTTTTT | | |
| SA-GFP | GATGGAGCC | 33 | GCAGCTCAGGTTCTGGGCTCAGAATTCCT | 34 | FIGS. |
| insertion | AGAGAGGAT | | TGTACAGCTCGTCCATGCCGAGAGTGATC | | 7C-7F |
| TJ-pegRNA | CC | | CCGGCGGCGGTCACGAACTCCAGCAGGAC | | |
| | | | CATGTGATCGCGCTTCTCGTTGGGGTCTT | | |
| | | | TGCTCAGGGCGGACTGGGTGCTCAGGTAG | | |
| | | | TGGTTGTCGGGCAGCAGCACGGGGCCGTC | | |
| | | | GCCGATGGGGGTGTTCTGCTGGTAGTGGT | | |
| | | | CGGCGAGCTGCACGCTGCCGTCCTCGATG | | |
| | | | TTGTGGCGGATCTTGAAGTTCACCTTGAT | | |
| | | | GCCGTTCTTCTGCTTGTCGGCCATGATAT | | |
| | | | AGACGTTGTGGCTGTTGTAGTTGTACTCC | | |
| | | | AGCTTGTGCCCCAGGATGTTGCCGTCCTC | | |
| | | | CTTGAAGTCGATGCCCTTCAGCTCGATGC | | |
| | | | GGTTCACCAGGGTGTCGCCCTCGAACTTC | | |
| | | | ACCTCGGCGCGGGTCTTGTAGTTGCCGTC | | |
| | | | GTCCTTGAAGAAGATGGTGCGCTCCTGGA | | |
| | | | CGTAGCCTTCGGGCATGGCGGACTTGAAG | | |
| | | | AAGTCGTGCTGCTTCATGTGGTCGGGGTA | | |
| | | | GCGGCTGAAGCACTGCACGCCGTAGGTCA | | |
| | | | GGGTGGTCACGAGGGTGGGCCAGGGCACG | | |
| | | | GGCAGCTTGCCGGTGGTGCAGATGAACTT | | |
| | | | CAGGGTCAGCTTGCCGTAGGTGGCATCGC | | |
| | | | CCTCGCCCTCGCCGGACACGCTGAACTTG | | |
| | | | TGGCCGTTTACGTCGCCGTCCAGCTCGAC | | |
| | | | CAGGATGGGCACCACCCCGGTGAACAGCT | | |
| | | | CCTCGCCCTTGCTCACTGGGCCAGGATTC | | |
| | | | TCCTCGACGTCACCGCATGTTAGCAGACT | | |
| | | | TCCTCTGCCCTCTCCGCTGCCAGATCTCT | | |
| | | | CGAGGCCCTGTGGGAGGAAGAGAAGAGGT | | |
| | | | CAGAAGCTTTCCTCTCTGGCTCTCTCTCT | | |
| | | | CTTGACGCGGTTCTATCTAGTTACGCGTT | | |
| | | | AAACCAACTAGAAATTTTTTT | | |
| AAVSI split | | 35 | AGACCCTCGACCGTCGATTGTCCACTGGT | 36 | FIGS. |
| circular TJ- | | | CAACAATAGATGACTTACAACTAATCGGA | | 8A-8E |
| pegRNA | | | AGGTGCAGAGACTCGACGGGAGCTACCCT | | |
| | | | AACGTCAAGACGAGGGTAAAGAGAGAGTC | | |
| | | | CAATTCTCAAAGCCAATAGGCAGTAGCGA | | |
| | | | AAGCTGCAAGAGAATGAAAATCCGTTGAC | | |
| | | | CTTAAACGGTCGTGTGGGTTCAAGTCCCT | | |
| | | | CCACCCCCACGCCGGAAACGCAATAGCCG | | |
| | | | AAAAACAAAAAAGCACATGAGGATCACCC | | |
| | | | ATGTGCGCAGCTCAGGTTCTGGGATAACT | | |
| | | | TCGTATAATGTATGCTATACGAAGTTATA | | |
| | | | ACAATCCCCCAACTGAGAGAACTCAAAGG | | |
| | | | TTACCCCAGTTGGGGCACATTACCCTGTT | | |
| | | | ATCCCTATCCTCTCTGGCTCCATCGTAAG | | |
| | | | CAAACCTTAGAGGTTCTGGCAAGCAAAAA | | |
| | | | AACAAAACGGCTATTATGCGTTACCGGCG | | |
| | | | AGACGCTACGGACTTAAATAATTGAGCCT | | |
| | | | TAAAGAAGAAATTCTTTAAGTGGATGCTC | | |
| | | | TCAAACTCAGGGAAACCTAAATCTAGTTA | | |
| | | | TAGACAAGGCAATCCTGAGCCAAGCCGAA | | |
| | | | GTAGTAATTAGTAAGACCAGTGGACAATC | | |
| | | | GACGGATAACAGCATATCTAG | | |
| Fah TJ-pegRNA | GTAGGCCCT | 37 | TAAGAACAGAACATCAGAGGAAGCTGGGC | 38 | FIG. 9B |
| | GGGAACAGA | | CACCAGGCATTACCGCTCCAGTCGTTCAT | | |
| | TT | | CAAAACCATTCCAAATATGTGCTCGTGAG | | |
| | | | CCTTGGAAATGGGAATGGGTTCACCGAAT | | |
| | | | CTGTTCCCAGGGCTATATATCTAGACGCG | | |
| | | | GTTCTATCTAGTTACGCGTTAAACCAACT | | |
| | | | AGAATTTTTTT | | |

| | | SEQ | |
|---|---|---|---|
| | | ID | |
| Nicking sgRNA | spacer sequence (5′-3′) | NO | Figure |
| AAVS1 nicking sgRNA | GCAGCTCAGGTTCTGGGAG | 39 | FIGS. 1B-1E, 7C-7F, 8A- |
| | A | | 8E |
| HEK3 nicking sgRNA | GTCAACCAGTATCCCGGTG | 40 | FIGS. 6A & 6B |
| | C | | |
| HEK3 control nicking | GCACATACTAGCCCCTGTC | 41 | FIGS. 6A & 6B |
| sgRNA | T | | |
| PRNP nicking sgRNA | GCATGTTTTCACGATAGTA | 42 | FIG. 6C |
| | A | | |
| IDS nicking sgRNA | ACTGAGGGATGTCTGAAGG | 43 | FIG. 6C |
| | C | | |
| TLR reporter nicking | TAGGTCAGGGTGGTCACGA | 44 | FIGS. 7A & 7B |
| sgRNA | | | |
| Fah nicking sgRNA | CCCTAAGAACAGAACATCA | 45 | FIGS. 9B |
| | G | | |

RC-PBS2 (bold font)-Insertion (italic)-PBS1 (bold italic)-Tevopreq1 (double underlined)

TABLE 2
Sequences of primers used for genomic DNA amplification.

| | | SEQ | | SEQ |
|---|---|---|---|---|
| | | ID | | ID |
| | F (5′-3′) | NO | R (5′-3′) | NO |
| AAVS1 | CTTGCCAGAACCTCTAAGGT | 46 | CCAGGATCAGTGAAACGCAC | 47 |
| HEK3 | TCTGCTGCAAGTAAGCATGCATTTG | 48 | GCCCCTTCCAGGGACCTC | 49 |
| PRNP | AGTAAGCCAAAAACCAACATG | 50 | CTGTACTCATCCATGGGCCT | 51 |
| IDS | ACGTTGAGCTGTGCAGAGAA | 52 | GTGCGTATGGAATAGCCCAT | 53 |

TABLE 3
Sequences of primers used for ddPCR.

| | | SEQ | | SEQ | | SEQ |
|---|---|---|---|---|---|---|
| | | ID | | ID | | ID |
| Gene | F (5′-3′) | NO | R (5′-3′) | NO | Probe | NO |
| AAVS1 | CTTGCCAGAACCTCTAAGGT | 54 | GCTGAACTTG | 55 | Ins CACCACCCCGGTGAACAGCTC | 56 |
| | | | TGGCCGTTT | | | |
| HEK3 | GCATGCATTTGTAGGCTTGATG | 57 | CAGCCAAACT | 58 | WT CCTGGCCTGGGTCAATCCTTGG | 59 |
| | | | TGTCAACCAG | | Ins CACGGGCAGCTTGCCGGTGG | 60 |
| | | | | | Ins CACCACCCCGGTGAACAGCTC | 61 |
| PRNP | F GTCAGTGGAACAAGCCGAGT | 62 | ACTTGGTTGG | 64 | WT TGAAGCACATGGCTGGTGCTGC | 65 |
| | F TAGGTCAGGGTGGTCACGAG | 63 | GGTAAC | | Ins CACGGGCAGCTTGCCGGTGG | 66 |
| | | | GGTG | | Ins CACCACCCCGGTGAACAGCTC | 67 |
| IDS | F GCTGAACTTGTGGCCGTTT | 68 | GTGCGTATGG | 70 | Ins CACCACCCCGGTGAACAGCTC | 71 |
| | F TAGGTCAGGGTGGTCACGAG | 69 | AATAGCCCAT | | Ins CACGGGCAGCTTGCCGGTGG | 72 |
| CCR5 | TAGGTCAGGGTGGTCACGAG | 73 | TTCCTGGGAG | 74 | Ins CACGGGCAGCTTGCCGGTGG | 75 |
| | | | AGACGCAAAC | | | |

TABLE 4
Sequences of primers used for high throughput sequencing.

| | | SEQ ID | | SEQ |
|---|---|---|---|---|
| Gene | F (5′-3′) | NO | R (5′-3′) | ID NO |
| AAVS1 | AGACGTGTGCTCTTCCGA | 76 | CTACACGACGCTCTTCCGA | 77 |
| | TCTCTTGCCAGAACCTCT | | TCTCCAGGATCAGTGAAAC | |
| | AAGGT | | GCAC | |
| Fah | CTACACGACGCTCTTCCG | 78 | AGACGTGTGCTCTTCCGAT | 79 |
| | ATCTACCAACTTTCTCCA | | CTTGTCCCATACCCAACTC | |
| | TGGCAG | | CTG | |

1. A prime editing guide RNA (pegRNA), comprising, from 5′ to 3′:
(1) a single guide RNA (sgRNA);
(2) a second primer binding sequence (2^{nd} PBS);
(3) an optional reverse transcription template (RTT) sequence; and,
(4) a first primer binding sequence (1^{st} PBS);
or a split variant combination (SVC) thereof, wherein the SVC comprises:
(a) the sgRNA; and,
(b) a prime editing template RNA (petRNA) comprising, from 5′ to 3′, (2)-(4), wherein the petRNA further comprises a linked aptamer (such as MS2) that specifically binds an aptamer binding protein (such as MCP or a functional fragment thereof that binds MS2);
wherein:
(i) the sgRNA is capable of forming a complex with a CRISPR/Cas nickase and targeting the complex to a target (e.g., target genomic) DNA sequence through base pairing with a targeting strand of the target (genomic) DNA sequence to enable nicking of the non-targeting strand reverse complementary to the targeting strand by the CRISPR/Cas nickase;
(ii) the 1^{st} PBS is capable of annealing with the 3′ end of the nicked non-targeting strand created by the CRISPR/Cas nickase, to prime reverse transcription of the RTT (if present) and the 2^{nd} PBS by a reverse transcriptase (RT); and,
(iii) the reverse transcription product of the 2^{nd} PBS is capable of annealing to an anchor sequence on the targeting strand, wherein nicking the targeting strand 3′ to the anchor sequence (e.g., by the CRISPR/Cas nickase and a nicking sgRNA) creates a 3′ end of the targeting strand capable of being extended by the RT to form a second strand cDNA, using the reverse transcribed RTT (if present) and the 1^{st} PBS as template;
or wherein:
(A) the sgRNA is capable of forming a complex with the CRISPR/Cas nickase and targeting the complex to the target (e.g., target genomic) DNA sequence through base pairing with the targeting strand of the target (genomic) DNA sequence to enable nicking of the non-targeting strand reverse complementary to the targeting strand by the CRISPR/Cas nickase;
(B) the 1^{st} PBS is capable of annealing with the 3′ end of the anchor sequence on the targeting strand (resulting from nicking by the CRISPR/Cas nickase and the nicking sgRNA) to prime reverse transcription of the RTT (if present) and the 2^{nd} PBS by the RT; and,
(C) the reverse transcription product of the 2^{nd} PBS is capable of annealing to the 3′ end of the nicked non-targeting strand created by the CRISPR/Cas nickase to enable the RT to synthesize a second strand cDNA, using the reverse transcribed RTT (if present) and the 1^{st} PBS as template;
wherein the reverse complement sequence of the anchor sequence on the non-targeting strand is either upstream (5′) or downstream (3′) of the 1^{st} PBS binding sequence;
optionally, the RT is fused to the CRISPR/Cas nickase, and/or optionally, the RT is fused to the aptamer binding protein.

2. The pegRNA or SVC of claim 1, wherein:
(a) the sgRNA is about 80-120 (e.g., 90-110, or about 100) nucleotides in length;
(b) the 1^{st} PBS is about 10-20 (e.g., 12-18 or about 15) nucleotides in length;
(c) the optional RTT is about 0-900 (e.g., 0-800, 0-850, 5-550, 10-500, 15-400, 20-300, 50-200, 30-60, 40-50, 80-150, 100-120, 0-5, 0, 100, 200, 300, 400, 500, 600, 700, 800, or about 900) nucleotides in length; and/or,
(d) the 2^{nd} PBS is about 10-20 (e.g., 12-18 or about 15) nucleotides in length.

3. The pegRNA or SVC of claim 1 or 2, further comprising a linker between the 1^{st} PBS and the RTT, between the RTT and the 2^{nd} PBS, and/or (in the pegRNA) between the 2^{nd} PBS and the sgRNA.

4. The pegRNA or SVC of claim 3, wherein the linker is independently 1, 2, 3, 4, 5, 6, 7, 8, 9, or 10 nucleotides in length.

5. The pegRNA or SVC of any one of claims 1-4, wherein the CRISPR/Cas nickase is a Class 2, Type II Cas effector enzyme (e.g., a Cas9, such as SpCas9, SpCas9-HF1, eSpCas9, SaCas9, SaCas9-HF, KKHSaCas9, StCas9, NmCas9, FnCas9, CjCas9, ScCas9, HypaCas9, xCas9, SpRY, SpG, or SauriCas9) lacking (HNH) endonuclease activity against the targeting strand.

6. The pegRNA or SVC of any one of claims 1-5, wherein the CRISPR/Cas nickase lacks endonuclease activity against the non-targeting strand, when forming a complex with the nicking sgRNA to nick the targeting strand (immediately) 3′ to the anchor sequence.

7. The pegRNA or SVC of any one of claims 1-6, wherein the nicking site of the non-targeting strand and the nicking site of the targeting strand are separated by 5, 10, 15, 20, 25, 30, 35, 40, 45, 50, 55, 60, 65, 70, 75, 80, 85, 90, 95, 100, 105, 110, 120, 130, 140, 150, 160, 170, 180, 190, 200, 210, 220, 230, 240, 250, 260, 270, 280, 290, or 300 nucleotides, with the nicking site of the non-targeting strand being either 5′ or 3′ to the nicking site of the targeting strand.

8. The pegRNA or SVC of any one of claims 1-7, wherein the 1^{st} PBS is linked to an RNA element that enhances pegRNA or petRNA stability, and/or improves prime editing efficiency; optionally, the RNA element comprises a trimmed evopreQ1 (tevopreQ1) motif or an aptamer such as MS2.

9. The pegRNA or SVC of any one of claims 1-8, wherein the petRNA is circular, and/or wherein the linked aptamer (such as MS2) is immediately 5′ to the 2^{nd} PBS.

10. The pegRNA or SVC of claim 9, wherein the circular petRNA is generated by in vitro transcription to generate a precursor RNA that is circularized post transcription via self-splicing through a permuted group I catalytic intron.

11. A prime editing guide RNA (pegRNA), comprising, from 5′ to 3′:
(1) a second primer binding sequence (2^{nd} PBS);
(2) an optional reverse transcription template (RTT) sequence;
(3) a first primer binding sequence (1^{st} PBS); and,
(4) a single guide RNA (sgRNA);
wherein:
(i) the sgRNA is capable of forming a complex with a CRISPR/Cas nickase and targeting the complex to a target (e.g., a target genomic) DNA sequence through base pairing with a targeting strand of the target (genomic) DNA sequence to enable nicking of the non-targeting strand reverse complementary to the targeting strand by the CRISPR/Cas nickase;
(ii) the 1^{st} PBS is capable of annealing with the 3′ end of the nicked non-targeting strand created by the CRISPR/Cas nickase, to prime reverse transcription of the RTT (if present) and the 2^{nd} PBS by a reverse transcriptase (RT);
optionally, the RT is fused to the CRISPR/Cas nickase; and,
(iii) the reverse transcription product of the 2^{nd} PBS is capable of annealing to an anchor sequence on the targeting strand, wherein nicking the targeting strand 3′ to the anchor sequence (e.g., by the CRISPR/Cas nickase and a nicking sgRNA) creates a 3′ end of the targeting strand capable of being extended by the RT to form a second strand cDNA, using the reverse transcribed RTT (if present) and the 1^{st} PBS as template;
or wherein:
(A) the sgRNA is capable of forming a complex with the CRISPR/Cas nickase and targeting the complex to the target (e.g., target genomic) DNA sequence through base pairing with the targeting strand of the target (genomic) DNA sequence to enable nicking of the non-targeting strand reverse complementary to the targeting strand by the CRISPR/Cas nickase;
(B) the 1^{st} PBS is capable of annealing with the 3′ end of the anchor sequence on the targeting strand (resulting from nicking by the CRISPR/Cas nickase and the nicking sgRNA) to prime reverse transcription of the RTT (if present) and the 2^{nd} PBS by the RT; and,
(C) the reverse transcription product of the 2^{nd} PBS is capable of annealing to the 3′ end of the nicked non-targeting strand created by the CRISPR/Cas nickase to enable the RT to synthesize a second strand cDNA, using the reverse transcribed RTT (if present) and the 1^{st} PBS as template;
wherein the reverse complement sequence of the anchor sequence on the non-targeting strand is either upstream (5′) or downstream (3′) of the 1^{st} PBS binding sequence.

12. The pegRNA of claim 11, wherein:
(a) the sgRNA is about 80-120 (e.g., 90-110, or about 100) nucleotides in length;
(b) the 1^{st} PBS is about 10-20 (e.g., 12-18 or about 15) nucleotides in length;
(c) the optional RTT is about 0-900 (e.g., 0-800, 0-850, 5-550, 10-500, 15-400, 20-300, 50-200, 30-60, 40-50, 80-150, 100-120, 0-5, 0, 100, 200, 300, 400, 500, 600, 700, 800, or about 900) nucleotides in length; and/or,
(d) the 2^{nd} PBS is about 10-20 (e.g., 12-18 or about 15) nucleotides in length.

13. The pegRNA of claim 11 or 12, further comprising a linker between the 1^{st} PBS and the RTT, between the RTT and the 2^{nd} PBS, and/or between the 2^{nd} PBS and the sgRNA.

14. The pegRNA of claim 13, wherein the linker is independently 1, 2, 3, 4, 5, 6, 7, 8, 9, or 10 nucleotides in length.

15. The pegRNA of any one of claims 11-14, wherein the CRISPR/Cas nickase is a Class 2, Type V Cas effector enzyme (e.g., Cas12a/Cpf1, Cas12b, Cas12c, Cas12d, Cas12e/CasX, Cas12f/Cas14, Cas12g, Cas12h, Cas12i, Cas12k, or V-U) lacking endonuclease activity against the targeting strand.

16. The pegRNA of any one of claims 11-15, wherein the CRISPR/Cas nickase lacks endonuclease activity against the non-targeting strand, when forming a complex with the nicking sgRNA to nick the targeting strand (immediately) 3′ to the anchor sequence.

17. The pegRNA of any one of claims 11-16, wherein the nicking site of the non-targeting strand and the nicking site of the targeting strand are separated by 5, 10, 15, 20, 25, 30, 35, 40, 45, 50, 55, 60, 65, 70, 75, 80, 85, 90, 95, 100, 105, 110, 120, 130, 140, 150, 160, 170, 180, 190, 200, 210, 220, 230, 240, 250, 260, 270, 280, 290, or 300 nucleotides, with the nicking site of the non-targeting strand being either 5′ or 3′ to the nicking site of the targeting strand.

18. A complex comprising:
(1) the pegRNA or SVC of any one of claims 1-10 (or the pegRNA of any one of claims 11-17); and,
(2) the CRISPR/Cas nickase of any one of claims 1-10 (or the pegRNA of any one of claims 11-17).

19. The complex of claim 18, further comprising:
(3) a target (e.g., a target genomic) DNA sequence, wherein the target (genomic) DNA sequence base pairs with the sgRNA through a targeting strand of the target (genomic) DNA sequence.

20. The complex of claim 19, further comprising:
(4) a reverse transcribed first strand cDNA reverse complementary in sequence to the 2^{nd} PBS and the RTT sequence (if present); and optionally,
(5) a reverse transcribed second strand cDNA reverse complementary in sequence to the first strand cDNA.

21. A method of inserting a donor DNA sequence into/around/proximate to a target (e.g., a target genomic) DNA sequence, the method comprising contacting the target (genomic) DNA sequence with: (1) the pegRNA or the SVC, (2) the CRISPR/Cas nickase, and (3) the nicking sgRNA, of any one of claims 1-10 (or 11-17), to permit the synthesis of a first strand cDNA and a second strand cDNA based on the RTT sequence of the pegRNA or SVC, through the reverse transcriptase (RT), wherein the RTT sequence encodes the donor DNA sequence.

22. The method of claim 21, wherein the method is carried out in vitro.

23. The method of claim 21, wherein the method is carried out in a cell.

24. The method of claim 23, wherein the cell is a eukaryotic cell, such as a mammalian cell (e.g., a human cell, or a rodent cell).

25. The method of claim 23 or 24, wherein the cell is within a live organism, such as a mammal (e.g., a human, a non-human mammal, a rodent, or a mouse).

26. The method of any one of claims 23-25, wherein (1) the pegRNA or SVC, (2) the CRISPR/Cas nickase, and/or (3) the nicking sgRNA is/are delivered to the cell via a vector or a non-vector delivery vehicle (such as nanoparticle).

27. The method of claim 26, wherein the vector is independently a plasmid, or a viral vector (e.g., an AAV vector, a lentiviral vector, or a retroviral vector).

28. The method of claim 27, wherein the AAV vector has a serotype of AAV1, AAV2, AAV3A, AAV3B, AAV4, AAV5, AAV6, AAV7, AAV8, AAV9, AAV10, AAV11, AAV12, AAV13, AAV-DJ, AAV PHP.eB, AAVrh74, or 7m8.

29. A polynucleotide comprising, from 5′ to 3′, (2)-(4) of any one of claims 1-10.

30. A polynucleotide encoding the pegRNA of any one of claims 1-17, the petRNA of any one of claims 1-10, or the polynucleotide of claim 29.

31. A vector comprising the polynucleotide of claim 30.

32. A cell comprising the polynucleotide of claim 30, or the vector of claim 31.

33. A pharmaceutical composition comprising the pegRNA, petRNA or SVC of any one of claims 1-17, the polynucleotide of claim 29 or 30, the vector of claim 31, or the cell of claim 32, and a pharmaceutically acceptable diluent or excipient.

34. A kit comprising the pegRNA, petRNA or SVC of any one of claims 1-17, the polynucleotide of claim 29 or 30, the vector of claim 31, or the cell of claim 32, and instructions for inserting a donor DNA sequence at a target DNA sequence.


SEQUENCE LISTING (96 sequences)

SEQ ID NO: 1 - DNA, 683 bp, synthetic construct
  misc_feature 1..683: note = Synthetic
  source 1..683: mol_type = other DNA
gatggagccagagaggatccgttttagagctagaaatagcaagttaaaataaggctagtccgttatcaacttgaaaaagtgggaccgagtcggtccgcagctcaggttctgggataacttcgtataatgtatgctatacgaagttataacaatcccccaactgagagaactcaaaggttaccccagttggggcacattaccctgttatccctacctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattcctctctggctctctctctcttgacgcggttctatctagttacgcgttaaaccaactagaaattttttt

SEQ ID NO: 2 - DNA, 20 bp, synthetic construct
  misc_feature 1..20: note = Synthetic
  source 1..20: mol_type = other DNA
ccaggatcagtgaaacgcac

SEQ ID NO: 3 - DNA, 20 bp, synthetic construct
  misc_feature 1..20: note = Synthetic
  source 1..20: mol_type = other DNA
cttgccagaacctctaaggt

SEQ ID NO: 4 - DNA, 76 bp, synthetic construct
  misc_feature 1..76: note = Synthetic
  source 1..76: mol_type = other DNA
gttttagagctagaaatagcaagttaaaataaggctagtccgttatcaacttgaaaaagtgggaccgagtcggtcc

SEQ ID NO: 5 - DNA, 20 bp, synthetic construct
  misc_feature 1..20: note = Synthetic
  source 1..20: mol_type = other DNA
gatggagccagagaggatcc

SEQ ID NO: 6 - DNA, 287 bp, synthetic construct
  misc_feature 1..287: note = Synthetic
  source 1..287: mol_type = other DNA
gcagctcaggttctgggataacttcgtataatgtatgctatacgaagttataacaatcccccaactgagagaactcaaaggttaccccagttggggcacattaccctgttatccctacctcgccggacacgctgaacttgtggccgtttacgtcgccgtccagctcgaccaggatgggcaccaccccggtgaacagctcctcgcccttgctcaccattcctctctggctctctctctcttgacgcggttctatctagttacgcgttaaaccaactagaaattttttt

SEQ ID NO: 7 - DNA, 20 bp, synthetic construct
  misc_feature 1..20: note = Synthetic
  source 1..20: mol_type = other DNA
gatggagccagagaggatcc

SEQ ID NO: 8 - DNA, 287 bp, synthetic construct
  misc_feature 1..287: note = Synthetic
  source 1..287: mol_type = other DNA
ccgggcaggtcacgcatataacttcgtataatgtatgctatacgaagttataacaatcccccaactgagagaactcaaaggttaccccagttggggcacattaccctgttatccctacctcgccggacacgctgaacttgtggccgtttacgtcgccgtccagctcgaccaggatgggcaccaccccggtgaacagctcctcgcccttgctcaccattcctctctggctctctctctcttgacgcggttctatctagttacgcgttaaaccaactagaaattttttt

SEQ ID NO: 9 - DNA, 20 bp, synthetic construct
  misc_feature 1..20: note = Synthetic
  source 1..20: mol_type = other DNA
gatggagccagagaggatcc

SEQ ID NO: 10 - DNA, 387 bp, synthetic construct
  misc_feature 1..387: note = Synthetic
  source 1..387: mol_type = other DNA
gcagctcaggttctgggataacttcgtataatgtatgctatacgaagttataacaatcccccaactgagagaactcaaaggttaccccagttggggcacattaccctgttatccctataggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattcctctctggctctctctctcttgacgcggttctatctagttacgcgttaaaccaactagaaattttttt

SEQ ID NO: 11 - DNA, 20 bp, synthetic construct
  misc_feature 1..20: note = Synthetic
  source 1..20: mol_type = other DNA
gatggagccagagaggatcc

SEQ ID NO: 12 - DNA, 387 bp, synthetic construct
  misc_feature 1..387: note = Synthetic
  source 1..387: mol_type = other DNA
ccgggcaggtcacgcatataacttcgtataatgtatgctatacgaagttataacaatcccccaactgagagaactcaaaggttaccccagttggggcacattaccctgttatccctataggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattcctctctggctctctctctcttgacgcggttctatctagttacgcgttaaaccaactagaaattttttt

SEQ ID NO: 13 - DNA, 20 bp, synthetic construct
  misc_feature 1..20: note = Synthetic
  source 1..20: mol_type = other DNA
gatggagccagagaggatcc

SEQ ID NO: 14 - DNA, 587 bp, synthetic construct
  misc_feature 1..587: note = Synthetic
  source 1..587: mol_type = other DNA
gcagctcaggttctgggataacttcgtataatgtatgctatacgaagttataacaatcccccaactgagagaactcaaaggttaccccagttggggcacattaccctgttatccctacctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattcctctctggctctctctctcttgacgcggttctatctagttacgcgttaaaccaactagaaattttttt

SEQ ID NO: 15 - DNA, 20 bp, synthetic construct
  misc_feature 1..20: note = Synthetic
  source 1..20: mol_type = other DNA
gatggagccagagaggatcc

SEQ ID NO: 16 - DNA, 587 bp, synthetic construct
  misc_feature 1..587: note = Synthetic
  source 1..587: mol_type = other DNA
ccgggcaggtcacgcatataacttcgtataatgtatgctatacgaagttataacaatcccccaactgagagaactcaaaggttaccccagttggggcacattaccctgttatccctacctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattcctctctggctctctctctcttgacgcggttctatctagttacgcgttaaaccaactagaaattttttt

SEQ ID NO: 17 - DNA, 20 bp, synthetic construct
  misc_feature 1..20: note = Synthetic
  source 1..20: mol_type = other DNA
ggcccagactgagcacgtga

SEQ ID NO: 18 - DNA, 282 bp, synthetic construct
  misc_feature 1..282: note = Synthetic
  source 1..282: mol_type = other DNA
gtcaaccagtatcccggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacgtgctcagtctgtagacacacgcggttctatctagttacgcgttaaaccaactagaattttttt

SEQ ID NO: 19 - DNA, 20 bp, synthetic construct
  misc_feature 1..20: note = Synthetic
  source 1..20: mol_type = other DNA
ggcccagactgagcacgtga

SEQ ID NO: 20 - DNA, 282 bp, synthetic construct
  misc_feature 1..282: note = Synthetic
  source 1..282: mol_type = other DNA
cctttcctctgccatcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacgtgctcagtctgtagacacacgcggttctatctagttacgcgttaaaccaactagaattttttt

SEQ ID NO: 21 - DNA, 20 bp, synthetic construct
  misc_feature 1..20: note = Synthetic
  source 1..20: mol_type = other DNA
gcagtggtggggggccttgg

SEQ ID NO: 22 - DNA, 287 bp, synthetic construct
  misc_feature 1..287: note = Synthetic
  source 1..287: mol_type = other DNA
gcatgttttcacgatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctaaggccccccaccatctctctcttgacgcggttctatctagttacgcgttaaaccaactagaaattttttt

SEQ ID NO: 23 - DNA, 20 bp, synthetic construct
  misc_feature 1..20: note = Synthetic
  source 1..20: mol_type = other DNA
gcagtggtggggggccttgg

SEQ ID NO: 24 - DNA, 287 bp, synthetic construct
  misc_feature 1..287: note = Synthetic
  source 1..287: mol_type = other DNA
ccagcatgtagccgccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctaaggccccccaccatctctctcttgacgcggttctatctagttacgcgttaaaccaactagaaattttttt

SEQ ID NO: 25 - DNA, 20 bp, synthetic construct
  misc_feature 1..20: note = Synthetic
  source 1..20: mol_type = other DNA
gcattttcgattccgtgact

SEQ ID NO: 26 - DNA, 287 bp, synthetic construct
  misc_feature 1..287: note = Synthetic
  source 1..287: mol_type = other DNA
actgagggatgtctgaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacacggaatcgaaatctctctcttgacgcggttctatctagttacgcgttaaaccaactagaaattttttt

SEQ ID NO: 27 - DNA, 20 bp, synthetic construct
  misc_feature 1..20: note = Synthetic
  source 1..20: mol_type = other DNA
gcattttcgattccgtgact

SEQ ID NO: 28 - DNA, 287 bp, synthetic construct
  misc_feature 1..287: note = Synthetic
  source 1..287: mol_type = other DNA
cggatcctcttccaagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacacggaatcgaaatctctctcttgacgcggttctatctagttacgcgttaaaccaactagaaattttttt

SEQ ID NO: 29 - DNA, 19 bp, synthetic construct
  misc_feature 1..19: note = Synthetic
  source 1..19: mol_type = other DNA
agttcagcgtgtccggctt

SEQ ID NO: 30 - DNA, 172 bp, synthetic construct
  misc_feature 1..172: note = Synthetic
  source 1..172: mol_type = other DNA
gtcagggtggtcacgagcgtaggccagggaacaggcagcttacccgttgtgcaaatgaatttgagtgtgagtttcccataggtggcatcgccctcgccctcgccggacacgctgataaatatcttgacgcggttctatctagttacgcgttaaaccaactagaaattttttt

SEQ ID NO: 31 - DNA, 20 bp, synthetic construct
  misc_feature 1..20: note = Synthetic
  source 1..20: mol_type = other DNA
gatggagccagagaggatcc

SEQ ID NO: 32 - DNA, 796 bp, synthetic construct
  misc_feature 1..796: note = Synthetic
  source 1..796: mol_type = other DNA
gcagctcaggttctgggtcaggcaccgggcttgcgggtcatgcaccaggtgcgcggtccttcgggcacctcgacgtcggcggtgacggtgaagccgagccgctcgtagaaggggaggttgtggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgcagtcgtccgcgacccacaccttgtcgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcattgggccaggattctcctcgacgtcaccgcatgttagcagacttcctctgccctctccgctgccagatctctcgaggccctgtgggaggaagagaagaggtcagaagctttcctctctggctctctctctcttgacgcggttctatctagttacgcgttaaaccaactagaaattttttt

SEQ ID NO: 33 - DNA, 20 bp, synthetic construct
  misc_feature 1..20: note = Synthetic
  source 1..20: mol_type = other DNA
gatggagccagagaggatcc

SEQ ID NO: 34 - DNA, 920 bp, synthetic construct
  misc_feature 1..920: note = Synthetic
  source 1..920: mol_type = other DNA
gcagctcaggttctgggctcagaattccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactgggccaggattctcctcgacgtcaccgcatgttagcagacttcctctgccctctccgctgccagatctctcgaggccctgtgggaggaagagaagaggtcagaagctttcctctctggctctctctctcttgacgcggttctatctagttacgcgttaaaccaactagaaattttttt

SEQ ID NO: 35 - skipped

SEQ ID NO: 36 - DNA, 630 bp, synthetic construct
  misc_feature 1..630: note = Synthetic
  source 1..630: mol_type = other DNA
agaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaagcacatgaggatcacccatgtgcgcagctcaggttctgggataacttcgtataatgtatgctatacgaagttataacaatcccccaactgagagaactcaaaggttaccccagttggggcacattaccctgttatccctatcctctctggctccatcgtaagcaaaccttagaggttctggcaagcaaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 37 - DNA, 20 bp, synthetic construct
  misc_feature 1..20: note = Synthetic
  source 1..20: mol_type = other DNA
gtaggccctgggaacagatt

SEQ ID NO: 38 - DNA, 185 bp, synthetic construct
  misc_feature 1..185: note = Synthetic
  source 1..185: mol_type = other DNA
taagaacagaacatcagaggaagctgggccaccaggcattaccgctccagtcgttcatcaaaaccattccaaatatgtgctcgtgagccttggaaatgggaatgggttcaccgaatctgttcccagggctatatatctagacgcggttctatctagttacgcgttaaaccaactagaattttttt

SEQ ID NO: 39 - DNA, 20 bp, synthetic construct
  misc_feature 1..20: note = Synthetic
  source 1..20: mol_type = other DNA
gcagctcaggttctgggaga

SEQ ID NO: 40 - DNA, 20 bp, synthetic construct
  misc_feature 1..20: note = Synthetic
  source 1..20: mol_type = other DNA
gtcaaccagtatcccggtgc

SEQ ID NO: 41 - DNA, 20 bp, synthetic construct
  misc_feature 1..20: note = Synthetic
  source 1..20: mol_type = other DNA
gcacatactagcccctgtct

SEQ ID NO: 42 - DNA, 20 bp, synthetic construct
  misc_feature 1..20: note = Synthetic
  source 1..20: mol_type = other DNA
gcatgttttcacgatagtaa

SEQ ID NO: 43 - DNA, 20 bp, synthetic construct
  misc_feature 1..20: note = Synthetic
  source 1..20: mol_type = other DNA
actgagggatgtctgaaggc

SEQ ID NO: 44 - DNA, 19 bp, synthetic construct
  misc_feature 1..19: note = Synthetic
  source 1..19: mol_type = other DNA
taggtcagggtggtcacga

SEQ ID NO: 45 - DNA, 20 bp, synthetic construct
  misc_feature 1..20: note = Synthetic
  source 1..20: mol_type = other DNA
ccctaagaacagaacatcag

SEQ ID NO: 46 - DNA, 20 bp, synthetic construct
  misc_feature 1..20: note = Synthetic
  source 1..20: mol_type = other DNA
cttgccagaacctctaaggt

SEQ ID NO: 47 - DNA, 20 bp, synthetic construct
  misc_feature 1..20: note = Synthetic
  source 1..20: mol_type = other DNA
ccaggatcagtgaaacgcac

SEQ ID NO: 48 - DNA, 25 bp, synthetic construct
  misc_feature 1..25: note = Synthetic
  source 1..25: mol_type = other DNA
tctgctgcaagtaagcatgcatttg

SEQ ID NO: 49 - DNA, 18 bp, synthetic construct
  misc_feature 1..18: note = Synthetic
  source 1..18: mol_type = other DNA
gccccttccagggacctc

SEQ ID NO: 50 - DNA, 21 bp, synthetic construct
  misc_feature 1..21: note = Synthetic
  source 1..21: mol_type = other DNA
agtaagccaaaaaccaacatg

SEQ ID NO: 51 - DNA, 20 bp, synthetic construct
  misc_feature 1..20: note = Synthetic
  source 1..20: mol_type = other DNA
ctgtactcatccatgggcct

SEQ ID NO: 52 - DNA, 20 bp, synthetic construct
  misc_feature 1..20: note = Synthetic
  source 1..20: mol_type = other DNA
acgttgagctgtgcagagaa

SEQ ID NO: 53 - DNA, 20 bp, synthetic construct
  misc_feature 1..20: note = Synthetic
  source 1..20: mol_type = other DNA
gtgcgtatggaatagcccat

SEQ ID NO: 54 - DNA, 20 bp, synthetic construct
  misc_feature 1..20: note = Synthetic
  source 1..20: mol_type = other DNA
cttgccagaacctctaaggt

SEQ ID NO: 55 - DNA, 19 bp, synthetic construct
  misc_feature 1..19: note = Synthetic
  source 1..19: mol_type = other DNA
gctgaacttgtggccgttt

SEQ ID NO: 56 - DNA, 21 bp, synthetic construct
  misc_feature 1..21: note = Synthetic
  source 1..21: mol_type = other DNA
caccaccccggtgaacagctc

SEQ ID NO: 57 - DNA, 22 bp, synthetic construct
  misc_feature 1..22: note = Synthetic
  source 1..22: mol_type = other DNA
gcatgcatttgtaggcttgatg

SEQ ID NO: 58 - DNA, 20 bp, synthetic construct
  misc_feature 1..20: note = Synthetic
  source 1..20: mol_type = other DNA
cagccaaacttgtcaaccag

SEQ ID NO: 59 - DNA, 22 bp, synthetic construct
  misc_feature 1..22: note = Synthetic
  source 1..22: mol_type = other DNA
cctggcctgggtcaatccttgg

SEQ ID NO: 60 - DNA, 20 bp, synthetic construct
  misc_feature 1..20: note = Synthetic
  source 1..20: mol_type = other DNA
cacgggcagcttgccggtgg

SEQ ID NO: 61 - DNA, 21 bp, synthetic construct
  misc_feature 1..21: note = Synthetic
  source 1..21: mol_type = other DNA
caccaccccggtgaacagctc

SEQ ID NO: 62 - DNA, 20 bp, synthetic construct
  misc_feature 1..20: note = Synthetic
  source 1..20: mol_type = other DNA
gtcagtggaacaagccgagt

SEQ ID NO: 63 - DNA, 20 bp, synthetic construct
  misc_feature 1..20: note = Synthetic
  source 1..20: mol_type = other DNA
taggtcagggtggtcacgag

SEQ ID NO: 64 - DNA, 20 bp, synthetic construct
  misc_feature 1..20: note = Synthetic
  source 1..20: mol_type = other DNA
acttggttggggtaacggtg

SEQ ID NO: 65 - DNA, 22 bp, synthetic construct
  misc_feature 1..22: note = Synthetic
  source 1..22: mol_type = other DNA
tgaagcacatggctggtgctgc

SEQ ID NO: 66 - DNA, 20 bp, synthetic construct
  misc_feature 1..20: note = Synthetic
  source 1..20: mol_type = other DNA
cacgggcagcttgccggtgg

SEQ ID NO: 67 - DNA, 21 bp, synthetic construct
  misc_feature 1..21: note = Synthetic
  source 1..21: mol_type = other DNA
caccaccccggtgaacagctc

SEQ ID NO: 68 - DNA, 19 bp, synthetic construct
  misc_feature 1..19: note = Synthetic
  source 1..19: mol_type = other DNA
gctgaacttgtggccgttt

SEQ ID NO: 69 - DNA, 20 bp, synthetic construct
  misc_feature 1..20: note = Synthetic
  source 1..20: mol_type = other DNA
taggtcagggtggtcacgag

SEQ ID NO: 70 - DNA, 20 bp, synthetic construct
  misc_feature 1..20: note = Synthetic
  source 1..20: mol_type = other DNA
gtgcgtatggaatagcccat

SEQ ID NO: 71 - DNA, 21 bp, synthetic construct
  misc_feature 1..21: note = Synthetic
  source 1..21: mol_type = other DNA
caccaccccggtgaacagctc

SEQ ID NO: 72 - DNA, 20 bp, synthetic construct
  misc_feature 1..20: note = Synthetic
  source 1..20: mol_type = other DNA
cacgggcagcttgccggtgg

SEQ ID NO: 73 - DNA, 20 bp, synthetic construct
  misc_feature 1..20: note = Synthetic
  source 1..20: mol_type = other DNA
taggtcagggtggtcacgag

SEQ ID NO: 74 - DNA, 20 bp, synthetic construct
  misc_feature 1..20: note = Synthetic
  source 1..20: mol_type = other DNA
ttcctgggagagacgcaaac

SEQ ID NO: 75 - DNA, 20 bp, synthetic construct
  misc_feature 1..20: note = Synthetic
  source 1..20: mol_type = other DNA
cacgggcagcttgccggtgg

SEQ ID NO: 76 - DNA, 41 bp, synthetic construct
  misc_feature 1..41: note = Synthetic
  source 1..41: mol_type = other DNA
agacgtgtgctcttccgatctcttgccagaacctctaaggt

SEQ ID NO: 77 - DNA, 42 bp, synthetic construct
  misc_feature 1..42: note = Synthetic
  source 1..42: mol_type = other DNA
ctacacgacgctcttccgatctccaggatcagtgaaacgcac

SEQ ID NO: 78 - DNA, 42 bp, synthetic construct
  misc_feature 1..42: note = Synthetic
  source 1..42: mol_type = other DNA
ctacacgacgctcttccgatctaccaactttctccatggcag

SEQ ID NO: 79 - DNA, 41 bp, synthetic construct
  misc_feature 1..41: note = Synthetic
  source 1..41: mol_type = other DNA
agacgtgtgctcttccgatcttgtcccatacccaactcctg

SEQ ID NO: 80 - protein, 7 aa, synthetic construct
  REGION 1..7: note = Synthetic
PKKKRKV

SEQ ID NO: 81 - protein, 18 aa, synthetic construct
  REGION 1..18: note = Synthetic
MKRTADGSEESPKKKRKV

SEQ ID NO: 82 - protein, 30 aa, synthetic construct
  REGION 1..30: note = Synthetic
MDSLLMNRRKFLYQFKNVRWAKGRRETYLC

SEQ ID NO: 83 - protein, 20 aa, synthetic construct
  REGION 1..20: note = Synthetic
AVKRPAATKKAGQAKKKKLD

SEQ ID NO: 84 - protein, 25 aa, synthetic construct
  REGION 1..25: note = Synthetic
MSRRRKANPTKLSENAKKLAKEVEN

SEQ ID NO: 85 - protein, 9 aa, synthetic construct
  REGION 1..9: note = Synthetic
PAAKRVKLD

SEQ ID NO: 86 - protein, 9 aa, synthetic construct
  REGION 1..9: note = Synthetic
KLKIKRPVK

SEQ ID NO: 87 - protein, 8 aa, synthetic construct
  REGION 1..8: note = Synthetic
VSRKRPRP

SEQ ID NO: 88 - protein, 10 aa, synthetic construct
  REGION 1..10: note = Synthetic
EGAPPAKRAR

SEQ ID NO: 89 - protein, 12 aa, synthetic construct
  REGION 1..12: note = Synthetic
PPQPKKKPLDGE

SEQ ID NO: 90 - protein, 21 aa, synthetic construct
  REGION 1..21: note = Synthetic
SGGSKRTADGSEFEPKKKRKV

SEQ ID NO: 91 - protein, 19 aa, synthetic construct
  REGION 1..19: note = Synthetic
NLSKRPAAIKKAGQAKKKK

SEQ ID NO: 92 - protein, 11 aa, synthetic construct
  REGION 1..11: note = Synthetic
RQRRNELKRSF

SEQ ID NO: 93 - protein, 38 aa, synthetic construct
  REGION 1..38: note = Synthetic
NQSSNFGPMKGGNFGGRSSGPYGGGGQYFAKPRNQGGY

SEQ ID NO: 94 - protein, 2117 aa, synthetic construct
  REGION 1..2117: note = Synthetic
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 95 - protein, 2117 aa, synthetic construct
  REGION 1..2117: note = Synthetic
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 96 - protein, 1943 aa, synthetic construct
  REGION 1..1943: note = Synthetic
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDNSRLINSGGSKRTADGSEFEPKKKRKV